(19) United States
(12) Patent Application Publication
(10) Pub. No.: US 2024/0024388 A1
(43) Pub. Date: Jan. 25, 2024

(54) PROBIOTIC COMPOSITIONS AND USES THEREOF

(71) Applicant: The University of British Columbia, Vancouver (CA)

(72) Inventors: Deanna Gibson, Kelowna (CA); Artem Godovannyi, Moscow (RU); Sandeep Gill, Kelowna (CA); Andrea Alejandra Verdugo-Meza, Kelowna (CA)

(21) Appl. No.: 18/317,071

(22) Filed: May 13, 2023

Related U.S. Application Data
(63) Continuation-in-part of application No. 16/486,803, filed on Aug. 16, 2019, now Pat. No. 11,684,643.
(60) Provisional application No. 62/460,185, filed on Feb. 17, 2017.

(51) Int. Cl. A61K 35/747; A61P 1/00; C07K 14/28

(52) U.S. Cl. CPC A61K 35/747; A61P 1/00; C07K 14/28

(57) ABSTRACT

The present invention relates to probiotic compositions. More specifically, the present invention relates to probiotic compositions that are useful in reducing inflammation and/or that exhibit increased colonization or persistence in the gastrointestinal tract of a mammal.

### CROSS-REFERENCE TO RELATED APPLICATION(S)

This application claims the benefit of U.S. patent application Ser. No. 16/486,803 entitled Probiotic Compositions and Uses Thereof, filed Aug. 16, 2019, which is a national phase application under 35 U.S.C. § 371 and claims priority to and the benefit of International Application No. PCT/CA2018/050188 filed Feb. 19, 2018, which claims priority to and the benefit of U.S. Provisional Application No. 62/460,185 filed Feb. 17, 2017, the entire contents of both of which are incorporated herein by reference.

### FIELD OF INVENTION

### Sequence Listing

The instant application contains a Sequence Listing submitted in XML format via the USPTO Patent Center on Oct. 4, 2023, and is incorporated herein by reference in its entirety. The XML file, created on Oct. 4, 2023, is named 1174-00-P01-US-CIP SEQ ID—amended and is 172,306 bytes in size.

The present invention relates to probiotic compositions. More specifically, the present invention relates to probiotic compositions that are useful in reducing inflammation.

### BACKGROUND OF THE INVENTION

Inflammatory bowel disease (IBD), inclusive of ulcerative colitis and Crohn's disease, is a major health burden in Western countries. A highly oxidized environment is found within the gastrointestinal tract during periods of inflammation. At sites of active inflammation within the gut of IBD patients, enhanced production of reactive oxygen species (ROS) is common. The abundance of these chemically-reactive species results in higher suppression of growth of anaerobic bacteria, compared to aerobic bacteria, because the latter are better adapted to ROS survival. The resulting overabundance of aerobic bacterial species is opposite to a healthy microbial ecosystem. This dysbiosis within the gastrointestinal system can have profound implications on human health. It is known that some aerobic bacteria residing within the gastrointestinal tract, such as Escherichia coli, induce damaging inflammation. Pathobiotic aerobic species, such as Adherent Invasive E. coli (AIEC), are associated with the mucosa of ulcerative colitis patients in elevated levels and cause damaging pro-inflammatory effects. This inflammation-induced disbalance is believed to further enhance inflammation and production of ROS.

Probiotic therapy, which is the ingestion of non-pathogenic microorganisms to provide health benefits, has been described as a potential treatment option for IBD. Probiotics are considered safe for human consumption, even in the absence of disease, as some have been shown to provide beneficial properties to the host. Various kinds of probiotics have been tested clinically as potential therapeutic agents for gut health. To date, however, many clinical trials have reported low efficacy of tested probiotic strains.

Probiotics face strong competition when trying to colonize the gut and very few studies have shown long-term colonization. Even adherent strains are diluted out by the existing microbiota unless replenished by a fresh inoculum of the strain. They are also not able to outcompete and face strong competition when trying to colonize and establish themselves (Alander M. et al. (1999) Appl Environ Microbiol. 65(1):351-354). Even in the absence of disease, there is a lack of evidence to support colonization of probiotics in healthy individuals.

Many of the commonly used probiotic species have a low tolerance to ROS. Aerobic probiotic species with anti-inflammatory effects (e.g. E. coli Nissle), may have additional colonization issues as inflammation sites are already heavily colonized with native gut aerobes and more invasive strains of E. coli (the effect commonly known as colonization resistance). The use of recombinant probiotic organisms that express bacterial virulence factors and stress survival genes from pathogenic bacteria (reviewed in Eamonn et al. 2009. Gut Pathogens November 23; 1(1):19) has been explored.

The N-acetyl-glucosamine binding protein (GbpA) from Vibrio cholerae is a well-studied adhesin with a modular multi-domain structure and studies have shown that the first N-terminal domain (GbpADI) is required for binding to intestinal epithelial cell (IEC)-associated mucins (Wong E et al. 2012 PLoS Pathog 8:e1002373.5).

Tetrathionate reductase is encoded as a 5-gene operon present in Salmonella ssp. and promotes the growth of Salmonella in the intestinal lumen during inflammation (Winter et al. (2010) Nature. 467:426-429). Studies have shown that the genes required for tetrathionate utilization can be expressed from a plasmid in E. coli giving its host the capacity to utilize tetrathionate (Hensel et al. 1999. Mol Microbiol. 32:275-287).

### SUMMARY OF THE INVENTION

The present invention relates, in part, to probiotic compositions.

In one aspect the present invention provides a recombinant probiotic bacterium expressing an N-acetyl-glucosamine binding protein A or fragment or homologue thereof.

In some embodiments, the N-acetyl-glucosamine binding protein A may include an amino acid sequence substantially identical to the sequence set forth in NCBI Accession No. KKP14471.

In some embodiments, the N-acetyl-glucosamine binding protein A may include an amino acid sequence substantially identical to SEQ ID NO: 19.

In some embodiments, the N-acetyl-glucosamine binding protein A may be encoded by a nucleic acid sequence substantially identical to SEQ ID NO: 26.

In some embodiments, the N-acetyl-glucosamine binding protein A fragment may be an N-terminal fragment.

In some embodiments, the N-terminal fragment may include a mucin binding domain.

In some embodiments, the mucin binding domain may be encoded by a nucleic acid sequence substantially identical to SEQ ID NO: 22.

In some embodiments, the N-terminal fragment may include an amino acid sequence substantially identical to SEQ ID NO: 20.

In some embodiments, the N-acetyl-glucosamine binding protein A may be encoded by a nucleic acid sequence substantially identical to SEQ ID NO: 27.

In some embodiments, the N-acetyl-glucosamine binding protein A may be encoded by a nucleic acid sequence harmonized for expression in a host microorganism.

In some embodiments, the N-acetyl-glucosamine binding protein A may be from a bacterium from the phyla Gammaproteobacteria, Enterobacteria or Firmicutes.

In some embodiments, the N-acetyl-glucosamine binding protein A may be from a Vibrio spp, Escherichia ssp., Yersinia ssp., Shewanella ssp., Photobacterium ssp., Listeria ssp., Enterobacter ssp., Aeromonas ssp., Klebsiella ssp. or Aliivibrio ssp.

In some embodiments, the N-acetyl-glucosamine binding protein A may be from a V. cholerae, V. mimicus, V. metoecus, V. vulnificus, V. parahaemolyticus, or V. fischeri.

In some embodiments, the N-acetyl-glucosamine binding protein A or fragment thereof may be co-expressed or recombined with a bacterial surface protein.

In some embodiments, the bacterial surface protein may be a mucus binding protein or a fragment thereof.

In some embodiments, the N-acetyl-glucosamine binding protein may include the mucus binding protein or a fragment thereof.

In some embodiments, the mucus binding protein may include an amino acid sequence substantially identical to SEQ ID NO: 21 or SEQ ID NO: 28.

In some embodiments, the recombined mucus binding protein-N-acetyl-glucosamine binding protein A may include an amino acid sequence substantially identical to SEQ ID NO: 29.

In some embodiments, the recombined mucus binding protein-N-acetyl-glucosamine binding protein A may be encoded by a nucleic acid sequence substantially identical to SEQ ID NO: 24 or SEQ ID NO: 30.

In some embodiments, the bacterial surface protein may be isolated from a non-pathogenic bacterium, such as a probiotic bacterium.

In some embodiments, the bacterial surface protein may be isolated from Lactobacillus reuteri.

In another aspect, the present invention provides a recombinant probiotic bacterium expressing a tetrathionate reductase or homologue thereof.

In some embodiments, the tetrathionate reductase may be encoded by the tetrathionate respiratory operon or portion thereof.

In some embodiments, the tetrathionate respiratory operon may include the ttrACBSR operon from Salmonella enterica.

In some embodiments, the ttrACBSR operon from Salmonella enterica may include a sequence substantially identical to SEQ ID NO: 25.

In some embodiments, the tetrathionate respiratory operon may include the ttrACB operon from Salmonella enterica.

In some embodiments, the ttrACB operon from Salmonella enterica may include a sequence substantially identical to SEQ ID NO: 31.

In some embodiments, the tetrathionate respiratory operon may further include the ttrSR operon from Salmonella enterica.

In some embodiments, the ttrSR operon from Salmonella enterica may include a sequence substantially identical to SEQ ID NO: 32.

In some embodiments, the tetrathionate reductase or the tetrathionate respiratory operon may include the ttrA, ttrC and ttrB genes of Salmonella enterica.

In some embodiments, the ttrA gene of Salmonella enterica may include a nucleic acid sequence substantially identical to SEQ ID NO: 33.

In some embodiments, the ttrA gene of Salmonella enterica may encode an amino acid sequence including a sequence substantially identical to SEQ ID NO: 34.

In some embodiments, the ttrB gene of Salmonella enterica may include a nucleic acid sequence substantially identical to SEQ ID NO: 35.

In some embodiments, the ttrB gene of Salmonella enterica may encode an amino acid sequence including a sequence substantially identical to SEQ ID NO: 36.

In some embodiments, the ttrC gene of Salmonella enterica may include a nucleic acid sequence substantially identical to SEQ ID NO: 37.

In some embodiments, the ttrC gene of Salmonella enterica may encode an amino acid sequence including a sequence substantially identical to SEQ ID NO: 38.

In some embodiments, the tetrathionate reductase may be encoded by, or the tetrathionate respiratory operon may further include, the ttrS and ttrR genes of Salmonella enterica.

In some embodiments, the ttrR gene of Salmonella enterica may include a nucleic acid sequence substantially identical to SEQ ID NO: 39.

In some embodiments, the ttrR gene of Salmonella enterica may encode an amino acid sequence including a sequence substantially identical to SEQ ID NO: 40.

In some embodiments, the ttrS gene of Salmonella enterica may include a nucleic acid sequence substantially identical to SEQ ID NO: 41.

In some embodiments, the ttrS gene of Salmonella enterica may encode an amino acid sequence including a sequence substantially identical to SEQ ID NO: 42.

In some embodiments, the ttrA, ttrC and ttrB genes of Salmonella enterica, or a tetrathionate respiratory operon including the ttrA, ttrC and ttrB genes of Salmonella enterica, may be provided in combination with an oxygen-sensitive promoter-operator.

In some embodiments, the tetrathionate reductase or homologue thereof may be isolated from the Enterobacteriaceae family or the Vibrionaceae family.

In some embodiments, the tetrathionate reductase or the tetrathionate respiratory operon may be isolated from a Salmonella ssp., Yersinia ssp., Proteus ssp., Citrobacter ssp., Klebsiella sp., Raoultella sp., Escherichia sp., Serratia sp., Leclercia sp., Morganella sp., Providencia sp. Enterobacter sp. or Vibrio sp.

In some embodiments, the tetrathionate reductase may be encoded by, or the tetrathionate respiratory operon may include, a nucleic acid sequence harmonized for expression in a host microorganism.

In some embodiments, the expression of the N-acetyl-glucosamine binding protein A, the tetrathionate reductase, or both, may be chromosomal or may be plasmid-based.

In some embodiments, the recombinant probiotic bacterium may be a Lactobacillus, Bifidobacterium, Lactococcus, Enterococcus, Streptococcus, Pediococcus, Leuconostoc, Bacillus, or Escherichia.

In some embodiments, the recombinant probiotic bacterium may be a Lactobacillus reuteri, Lactobacillus plantarum, Lactobacillus casei, Lactobacillus casei Shirota, Lactobacillus salivarius, Lactobacillus paracasei, Lactobacillus lactis, Lactobacillus acidophilus, Lactobacillus sakei, Lactobacillus brevis, Lactobacillus buchneri, Lactobacillus fermentum, Lactobacillus delbrueckii, Lactobacillus delbrueckii subsp. bulgaricus, Lactobacillus helveticus, Lactobacillus garvieae, Lactobacillus acetotolerans, Lactobacillus agilis, Lactobacillus algidus, Lactobacillus alimentarius, Lactobacillus amylolyticus, Lactobacillus amylophilus, Lactobacillus amylovorus, Lactobacillus animalis, Lactobacillus aviarus, Lactobacillus bifermentans, Lactobacillus bulgaricus, Lactobacillus carnis, Lactobacillus caternaformis, Lactobacillus cellobiosis, Lactobacillus collinoides, Lactobacillus confuses, Lactobacillus coryniformis, Lactobacillus crispatus, Lactobacillus curvatus, Lactobacillus divergens, Lactobacillus farciminis, Lactobacillus fructivorans, Lactobacillus fructosus, Lactobacillus gallinarum, Lactobacillus gasseri, Lactobacillus graminis, Lactobacillus halotolerans, Lactobacillus hamster, Lactobacillus heterohiochii, Lactobacillus hilgardii, Lactobacillus homohiochii, Lactobacillus iners, Lactobacillus intestinalis, Lactobacillus jensenii, Lactobacillus johnsonii, Lactobacillus kandleri, Lactobacillus kefiri, Lactobacillus kefuranofaciens, Lactobacillus kefirgranum, Lactobacillus kunkeei, Lactobacillus leichmannii, Lactobacillus lindneri, Lactobacillus malefermentans, Lactobacillus mali, Lactobacillus maltaromicus, Lactobacillus manihotivorans, Lactobacillus minor, Lactobacillus minutus, Lactobacillus mucosae, Lactobacillus murinus, Lactobacillus nagelii, Lactobacillus oris, Lactobacillus panis, Lactobacillus parabuchneri, Lactobacillus paracasei, Lactobacillus parakefiri, Lactobacillus paralimentarius, Lactobacillus paraplantarum, Lactobacillus pentosus, Lactobacillus perolens, Lactobacillus piscicola, Lactobacillus plantarum, Lactobacillus pontis, Lactobacillus rhamnosus, Lactobacillus rhamnosus GG, Lactobacillus rimae, Lactobacillus rogosae, Lactobacillus ruminis, Lactobacillus sanfranciscensis, Lactobacillus sharpeae, Lactobacillus suebicus, Lactobacillus trichodes, Lactobacillus uli, Lactobacillus vaccinostercus, Lactobacillus vaginalis, Lactobacillus viridescens, Lactobacillus vitulinus, Lactobacillus xylosus, Lactobacillus yamanashiensis, or a Lactobacillus zeae, Escherichia coli, Bifidobacterium infantis, Bifidobacterium adolescentis, Bifidobacterium animalis subsp animalis, Bifidobacterium longum, Bifidobacterium breve, Bifidobacterium bifidum, Bifidobacterium animalis subsp. Lactis, Bifidobacterium lactis, Bifidobacterium lactis DN-173 010, Bacillus coagulans, Lactococcus lactis subsp. Lactis, Lactococcus lactis subsp. lactis CV56, Enterococcus durans, or Streptococcus thermophilus.

In some embodiments, the recombinant probiotic bacterium may be E. coli Nissle 1917 or L. reuteri DSM20016.

In some embodiments, the recombinant probiotic bacterium may include an auxotrophic mutation.

In alternative aspects, the present invention provides a nucleic acid molecule including a nucleic acid sequence encoding an N-acetyl-glucosamine binding protein A or fragment or homologue thereof in combination with a bacterial surface protein.

In some embodiments, the N-acetyl-glucosamine binding protein A or fragment or homologue thereof may be encoded by a nucleic acid sequence substantially identical to SEQ ID NO: 22, SEQ ID NO: 26 or SEQ ID NO: 27.

In some embodiments, the nucleic acid molecule may encode a N-acetyl-glucosamine binding protein A or fragment or homologue thereof including an amino acid sequence substantially identical to SEQ ID NO: 19 or SEQ ID NO: 20.

In some embodiments, the bacterial surface protein may be a mucus binding protein or homologue thereof.

In some embodiments, the mucus binding protein may be encoded by a nucleic acid sequence substantially identical to SEQ ID NO: 23 or SEQ ID NO: 53.

In some embodiments, the nucleic acid molecule may encode a mucus binding protein or homologue thereof including an amino acid sequence substantially identical to SEQ ID NO: 21 or SEQ ID NO: 28.

In some embodiments, the nucleic acid molecule may include a sequence substantially identical to SEQ ID NO: 24 or SEQ ID NO: 30.

In alternative aspects, the present invention provides a vector including a nucleic acid sequence as described herein.

In some embodiments, the vector may include a sequence substantially identical to SEQ ID NO: 44.

In alternative aspects, the present invention provides a host cell including a vector as described herein.

In alternative aspects, the present invention provides a method of increasing colonization of a probiotic bacterium in the gastrointestinal tract of a subject in need thereof, by administering a recombinant probiotic bacterium as described herein to the subject.

In alternative aspects, the present invention provides a method of reducing inflammation in the gastrointestinal tract of a subject in need thereof, by administering a recombinant probiotic bacterium as described herein to the subject.

In alternative aspects, the present invention provides a method of treating or preventing inflammatory bowel disease in a subject in need thereof, by administering a recombinant probiotic bacterium as described herein to the subject.

In alternative aspects, the present invention provides a use of the recombinant probiotic bacterium as described herein, for increasing colonization of a probiotic bacterium in the gastrointestinal tract of a subject in need thereof.

In alternative aspects, the present invention provides a use of the recombinant probiotic bacterium as described herein, for reducing inflammation in the gastrointestinal tract of a subject in need thereof.

In alternative aspects, the present invention provides a use of the recombinant probiotic bacterium as described herein, for treating or preventing inflammatory bowel disease in a subject in need thereof.

In some embodiments, the subject may be a human.

This summary of the invention does not necessarily describe all features of the invention.

### BRIEF DESCRIPTION OF THE DRAWINGS

These and other features of the invention will become more apparent from the following description in which reference is made to the appended drawings.

FIG. 1 is a schematic representation of the multi-domain structure of Mucus Binding Protein of Lactobacillus reuteri DSM20016, consisting of several Mucus Binding Domains (MBD) before and after proposed modification.

FIG. 2A is a photograph showing the macroscopic examination of cecum and colon from a mouse treated with a parental probiotic strain (E. coli Nissle attB^{phi80}::Km^{R}) followed by exposure to 3.5% DSS to induce colitis.

FIG. 2B is a second photograph showing the macroscopic examination of cecum and colon from a mouse treated with a parental probiotic strain (E. coli Nissle attB^{phi80}::Km^{R}) followed by exposure to 3.5% DSS to induce colitis.

FIG. 2C is a photograph showing the macroscopic examination of cecum and colon from a mouse treated with a recombinant probiotic strain expressing the ttr operon (E. coli Nissle attB^{phi80}::ttrACBSR) followed by exposure to 3.5% DSS to induce colitis.

FIG. 2D is a second photograph showing the macroscopic examination of cecum and colon from a mouse treated with a recombinant probiotic strain expressing the ttr operon (E. coli Nissle attB^{phi80}::ttrACBSR) followed by exposure to 3.5% DSS to induce colitis.

FIG. 3A is a photograph showing the macroscopic examination of cecum and colon from a mouse treated with a parental probiotic strain (L. reuteri) followed by exposure to 3.5% DSS to induce colitis.

FIG. 3B is a second photograph showing the macroscopic examination of cecum and colon from a mouse treated with a parental probiotic strain (L. reuteri) followed by exposure to 3.5% DSS to induce colitis.

FIG. 3C is a photograph showing the macroscopic examination of cecum and colon from a mouse treated with a recombinant probiotic strain expressing the recombinant probiotic (L. reuteri::GbpA) followed by exposure to 3.5% DSS to induce colitis.

FIG. 3D is a second photograph showing the macroscopic examination of cecum and colon from a mouse treated with a recombinant probiotic strain expressing the recombinant probiotic (L. reuteri::GbpA) followed by exposure to 3.5% DSS to induce colitis.

FIG. 4A is a graph showing the weight loss during the DSS treatment period for mice treated with the parental probiotic strain (E. coli Nissle), labelled as Parent Strain (Squares). Circles represent the group of mice treated with the recombinant probiotic strain, labelled as the Designer Strain (E. coli Nissle attB^{phi80}::ttrACBSR). Triangles represent the no probiotic DSS control. Weight loss calculated as percentage of weight loss from starting body weight prior to DSS exposure. Values are expressed as mean+/−SEM (n=4-8). Non-parametric one-way ANOVA (Kruskal-Wallis) was used.

FIG. 4B is a graph showing the weight loss during the DSS treatment period for mice treated with the parental probiotic strain (L. reuteri), labelled as Parent Strain (Squares). Circles represent the group of mice treated with the recombinant probiotic strain, labelled as the Designer Strain (L. reuteri::GbpA). Triangles represent the no probiotic DSS control. Weight loss calculated as percentage of weight loss from starting body weight prior to DSS exposure. Values are expressed as mean+/−SEM (n=4-8).

FIG. 5A is a graph showing clinical scores following DSS-induced colitis in mice pre-treated with the parental probiotic strain (E. coli Nissle), labelled as Parent Strain (Squares). Circles represent the group of mice treated with the recombinant probiotic strain, labelled as the Designer Strain (E. coli Nissle attB^{phi80}::ttrACBSR). Triangles represent the no probiotic DSS control. Movement, rectal bleeding, stool consistency, weight loss, and hydration were used to calculate clinical scores. Values expressed as mean+/−SEM (n=4-8). Non-parametric one-way ANOVA (Kruskal-Wallis) was used.

FIG. 5B is a graph showing clinical scores following DSS-induced colitis in mice pre-treated with the parental probiotic strain (L. reuteri), labelled as Parent Strain (Squares). Circles represent the group of mice treated with the recombinant probiotic strain, labelled as the Designer Strain (L. reuteri::GbpA). Triangles represent the no probiotic DSS control. Movement, rectal bleeding, stool consistency, weight loss, and hydration were used to calculate clinical scores. Values expressed as mean+/−SEM (n=4-8).

FIG. 6A is a graph showing the inflammatory cytokine TNF-α profile after DSS exposure for mice pre-treated with either the parental probiotic strain (E. coli Nissle 1917), labelled as Parent Strain, the recombinant probiotic strain (E. coli Nissle attB^{phi80}::ttrACBSR) labelled as Designer Strain, or a no-probiotic DSS control. Non-parametric one-way ANOVA (Kruskal-Wallis) was used.

FIG. 6B is a graph showing the inflammatory cytokine IFN-γ profile after DSS exposure for mice pre-treated with either the parental probiotic strain (E. coli Nissle 1917), labelled as Parent Strain, the recombinant probiotic strain (E. coli Nissle attB^{phi80}::ttrACBSR) labelled as Designer Strain, or a no-probiotic DSS control. Non-parametric one-way ANOVA (Kruskal-Wallis) was used.

FIG. 6C is a graph showing the inflammatory cytokine IL-1β profile after DSS exposure for mice pre-treated with either the parental probiotic strain (E. coli Nissle 1917), labelled as Parent Strain, the recombinant probiotic strain (E. coli Nissle attB^{phi80}::ttrACBSR) labelled as Designer Strain, or a no-probiotic DSS control. Non-parametric one-way ANOVA (Kruskal-Wallis) was used.

FIG. 6D is a graph showing the inflammatory cytokine IL-17a profile after DSS exposure for mice pre-treated with either the parental probiotic strain (E. coli Nissle 1917), labelled as Parent Strain, the recombinant probiotic strain (E. coli Nissle attB^{phi80}::ttrACBSR) labelled as Designer Strain, or a no-probiotic DSS control. Non-parametric one-way ANOVA (Kruskal-Wallis) was used.

FIG. 7A is a graph showing the inflammatory cytokine TNF-α profile after DSS exposure for mice pre-treated with either the parental probiotic strain (L. reuteri), labelled as Parent Strain, the recombinant probiotic strain (L. reuteri::GbpA) labelled as Designer Strain, or the no probiotic DSS control. Non-parametric one-way ANOVA (Kruskal-Wallis) was used.

FIG. 7B is a graph showing the inflammatory cytokine IFN-γ profile after DSS exposure for mice pre-treated with either the parental probiotic strain (L. reuteri), labelled as Parent Strain, the recombinant probiotic strain (L. reuteri::GbpA) labelled as Designer Strain, or the no probiotic DSS control. Non-parametric one-way ANOVA (Kruskal-Wallis) was used.

FIG. 7C is a graph showing the inflammatory cytokine IL-1β profile after DSS exposure for mice pre-treated with either the parental probiotic strain (L. reuteri), labelled as Parent Strain, the recombinant probiotic strain (L. reuteri::GbpA) labelled as Designer Strain, or the no probiotic DSS control. Non-parametric one-way ANOVA (Kruskal-Wallis) was used.

FIG. 7D is a graph showing the inflammatory cytokine IL-17a profile after DSS exposure for mice pre-treated with either the parental probiotic strain (L. reuteri), labelled as Parent Strain, the recombinant probiotic strain (L. reuteri::GbpA) labelled as Designer Strain, or the no probiotic DSS control. Non-parametric one-way ANOVA (Kruskal-Wallis) was used.

FIG. 8 is a graph showing the growth advantage of E. coli Nissle attB^{phi80}::ttrACBSR in growth competition with wild type E. coli Nissle in the presence of tetrathionate. Difference in the percent of tetracycline resistant colony forming units (CFUs) is related to growth advantage of E. coli Nissle attB^{phi80}::ttrACBSR in the presence of tetrathionate.

FIG. 9 is a graph showing thiosulfate production by wild type E. coli Nissle or the designer E. coli Nissle attB^{phi80}::ttrACBSR strain under oxic or anoxic conditions. All strains were grown in tetrathionate-containing media and consumption of tetrathionate was estimated based on conversion of tetrationate to thiosulfate.

FIG. 10A is a graph showing weight loss during DSS treatment period for mice treated with the parental probiotic strain (E. coli Nissle), labelled as Parent Strain (Squares). Circles represent the group of mice treated with the recombinant probiotic strain, labelled as the Designer Strain (E. coli Nissle attB^{phi80}::ttrACBSR). Triangles represent the no probiotic DSS control. Weight loss was calculated as percentage of weight loss from starting body weight prior to DSS exposure. Values are expressed as mean+/−SEM (n=10-12).

FIG. 10B is a graph showing clinical scores following DSS-induced colitis in mice pre-treated with the parental probiotic strain (E. coli Nissle), labelled as Parent Strain (Squares). Circles represent the group of mice treated with the recombinant probiotic strain, labelled as the Designer Strain (E. coli Nissle attB^{phi80}::ttrACBSR). Triangles represent the no probiotic DSS control. Movement, rectal bleeding, stool consistency, weight loss, and hydration were used to calculate clinical scores. Values expressed as mean+/−SEM (n=10-12). Non-parametric one-way ANOVA (Kruskal-Wallis) was used.

FIG. 11A is a graph showing the weight loss during the DSS treatment period for mice treated with parental probiotic (L. reuteri), labeled as Parent Strain (Squares). Circles represent the group of mice treated with the recombinant probiotic strain, labeled as the Designer Strain (L. reuteri::GbpA). Triangles represent the no probiotic DSS control Weight loss calculated as percentage of weight loss from starting body weight. Values are expressed as mean+/−SEM (n=10-12).

FIG. 11B is a graph showing clinical scores following DSS-induced colitis in mice pre-treated with the parental probiotic strain (L. reuteri), labelled as Parent Strain (Squares). Circles represent the group of mice treated with the recombinant probiotic strain, labeled as the Designer Strain (L. reuteri::GbpA). Triangles represent the no probiotic DSS control. Movement, rectal bleeding, stool consistency, weight loss, and hydration were used to calculate clinical scores. Values expressed as mean+/−SEM (n=10-12). Non-parametric one-way ANOVA (Kruskal-Wallis) was used.

FIG. 12A is a graph showing the total histopathological scores of the DSS control, E. coli parent, and E. coli designer strains. H&E stained slides of cross sections of the distal colon were used to calculate histopathological scores. Epithelial integrity, immune cell infiltration, ulceration, and goblet cell depletion were used to calculate histopathological scores in cross sections of the distal colon. Values are expressed as mean+/−SEM (n=10-12). Non-parametric one-way ANOVA (Kruskal-Wallis) test was used.

FIG. 12B shows representative images of H&E stained slides of cross sections of the distal colon of DSS-induced colitis mice used to calculate histopathological scores of the DSS control. H&E stained slides of cross sections of the distal colon were used to calculate histopathological scores. Epithelial integrity, immune cell infiltration, ulceration, and goblet cell depletion were used to calculate histopathological scores in cross sections of the distal colon. Values are expressed as mean+/−SEM (n=10-12). Non-parametric one-way ANOVA (Kruskal-Wallis) test was used.

FIG. 12C shows representative images of H&E stained slides of cross sections of the distal colon of E. coli DSS-induced colitis mice used to calculate histopathological scores of the E. coli parent strain. H&E stained slides of cross sections of the distal colon were used to calculate histopathological scores. Epithelial integrity, immune cell infiltration, ulceration, and goblet cell depletion were used to calculate histopathological scores in cross sections of the distal colon. Values are expressed as mean+/−SEM (n=10-12). Non-parametric one-way ANOVA (Kruskal-Wallis) test was used.

FIG. 12D shows representative images of H&E stained slides of cross sections of the distal colon of E. coli DSS-induced colitis mice used to calculate histopathological scores of the E. coli designer strain. H&E stained slides of cross sections of the distal colon were used to calculate histopathological scores. Epithelial integrity, immune cell infiltration, ulceration, and goblet cell depletion were used to calculate histopathological scores in cross sections of the distal colon. Values are expressed as mean+/−SEM (n=10-12). Non-parametric one-way ANOVA (Kruskal-Wallis) test was used.

FIG. 13A is a graph showing the total histopathological of the DSS control, L. reuteri parent, and L. reuteri designer strains. H&E stained slides of cross sections of the distal colon were used to calculate histopathological scores. Epithelial integrity, immune cell infiltration, ulceration, and goblet cell depletion were used to calculate histopathological scores. Values are expressed as mean+/−SEM (n=10-12). Non-parametric one-way ANOVA (Kruskal-Wallis) test was used.

FIG. 13B shows representative images of H&E stained slides of cross sections of the distal colon of DSS-induced colitis mice used to calculate histopathological scores of the DSS control. H&E stained slides of cross sections of the distal colon were used to calculate histopathological scores. Epithelial integrity, immune cell infiltration, ulceration, and goblet cell depletion were used to calculate histopathological scores. Values are expressed as mean+/−SEM (n=10-12). Non-parametric one-way ANOVA (Kruskal-Wallis) test was used.

FIG. 13C shows representative images of H&E stained slides of cross sections of the distal colon of L. reuteri DSS-induced colitis mice used to calculate histopathological scores of the L. reuteri parent strain. H&E stained slides of cross sections of the distal colon were used to calculate histopathological scores. Epithelial integrity, immune cell infiltration, ulceration, and goblet cell depletion were used to calculate histopathological scores. Values are expressed as mean+/−SEM (n=10-12). Non-parametric one-way ANOVA (Kruskal-Wallis) test was used.

FIG. 13D shows representative images of H&E stained slides of cross sections of the distal colon of L. reuteri DSS-induced colitis mice used to calculate histopathological scores of the L. reuteri designer strain. H&E stained slides of cross sections of the distal colon were used to calculate histopathological scores. Epithelial integrity, immune cell infiltration, ulceration, and goblet cell depletion were used to calculate histopathological scores. Values are expressed as mean+/−SEM (n=10-12). Non-parametric one-way ANOVA (Kruskal-Wallis) test was used.

FIG. 14A is a graph showing macrophage colonic cell infiltration (F4/80 positive cells per mouse tissue section) in DSS-induced colitis mice pre-treated with either the E. coli Parent strain, E. coli Designer strain or the DSS control. Positive cells were quantified in the sub-mucosal lamina propria region on stained tissues via immunofluorescence. Values are expressed as mean+/−SEM (n=10-12). Non-parametric one-way ANOVA (Kruskal-Wallis) test was used.

FIG. 14B is an immunofluorescence stained slide of the distal colon of DSS-induced colitis mice, showing a representative image of colonic cell infiltration in the sub-mucosal region on a cross sectional cut slide of the distal colon of the DSS control. Positive cells were quantified in the sub-mucosal lamina propria region on stained tissues via immunofluorescence. Values are expressed as mean+/−SEM (n=10-12). Non-parametric one-way ANOVA (Kruskal-Wallis) test was used.

FIG. 14C is an immunofluorescence stained slide of the distal colon of E. coli DSS-induced colitis mice, showing a representative image of colonic cell infiltration in the sub-mucosal region on a cross sectional cut slide of the distal colon of the E. coli parent strain. Positive cells were quantified in the sub-mucosal lamina propria region on stained tissues via immunofluorescence. Values are expressed as mean+/−SEM (n=10-12). Non-parametric one-way ANOVA (Kruskal-Wallis) test was used.

FIG. 14D is an immunofluorescence stained slide of the distal colon of E. coli DSS-induced colitis mice, showing a representative image of colonic cell infiltration in the sub-mucosal region on a cross sectional cut slide of the distal colon of the E. coli designer strain. Positive cells were quantified in the sub-mucosal lamina propria region on stained tissues via immunofluorescence. Values are expressed as mean+/−SEM (n=10-12). Non-parametric one-way ANOVA (Kruskal-Wallis) test was used.

FIG. 15A is a graph showing macrophage colonic cell infiltration (F4/80 positive cells per mouse tissue section) in DSS-induced colitis mice that were administered either the L. reuteri Parent strain, L. reuteri Designer strain or the DSS control. Positive cells were quantified in the sub-mucosal lamina propria region on stained tissues via immunofluorescence. Values are expressed as mean+/−SEM (n=10-12). Non-parametric one-way ANOVA (Kruskal-Wallis) test was used.

FIG. 15B is an immunofluorescence stained slide of the distal colon of DSS-induced colitis mice, showing a representative image of colonic cell infiltration in the sub-mucosal region on a cross sectional cut slide of the distal colon of the DSS control. Positive cells were quantified in the sub-mucosal lamina propria region on stained tissues via immunofluorescence. Values are expressed as mean+/−SEM (n=10-12). Non-parametric one-way ANOVA (Kruskal-Wallis) test was used.

FIG. 15C is an immunofluorescence stained slide of the distal colon of L. reuteri DSS-induced colitis mice, showing a representative image of colonic cell infiltration in the sub-mucosal region on a cross sectional cut slide of the distal colon of the L. reuteri parent strain. Positive cells were quantified in the sub-mucosal lamina propria region on stained tissues via immunofluorescence. Values are expressed as mean+/−SEM (n=10-12). Non-parametric one-way ANOVA (Kruskal-Wallis) test was used.

FIG. 15D is an immunofluorescence stained slide of the distal colon of L. reuteri DSS-induced colitis mice, showing a representative image of colonic cell infiltration in the sub-mucosal region on a cross sectional cut slide of the distal colon of the L. reuteri designer strain. Positive cells were quantified in the sub-mucosal lamina propria region on stained tissues via immunofluorescence. Values are expressed as mean+/−SEM (n=10-12). Non-parametric one-way ANOVA (Kruskal-Wallis) test was used.

FIG. 16A is a graph showing neutrophil colonic cell infiltration (MPO positive cells per mouse tissue section) in DSS-induced colitis mice that were administered with the E. coli Parent strain, E. coli Designer strain or the DSS control. Positive cells were quantified in the sub-mucosal lamina propria region on stained tissues via immunofluorescence. Values are expressed as mean+/−SEM (n=10-12). Non-parametric one-way ANOVA (Kruskal-Wallis) test was used.

FIG. 16B is an immunofluorescence stained slide of the distal colon of DSS-induced colitis mice showing a representative image of colonic cell infiltration in the sub-mucosal region on a cross sectional cut slide of the distal colon of the DSS control. Positive cells were quantified in the sub-mucosal lamina propria region on stained tissues via immunofluorescence. Values are expressed as mean+/−SEM (n=10-12). Non-parametric one-way ANOVA (Kruskal-Wallis) test was used.

FIG. 16C is an immunofluorescence stained slide of the distal colon of E. coli DSS-induced colitis mice showing a representative image of colonic cell infiltration in the sub-mucosal region on a cross sectional cut slide of the distal colon of the E. coli parent strain. Positive cells were quantified in the sub-mucosal lamina propria region on stained tissues via immunofluorescence. Values are expressed as mean+/−SEM (n=10-12). Non-parametric one-way ANOVA (Kruskal-Wallis) test was used.

FIG. 16D is an immunofluorescence stained slide of the distal colon of E. coli DSS-induced colitis mice showing a representative image of colonic cell infiltration in the sub-mucosal region on a cross sectional cut slide of the distal colon of the E. coli designer strain. Positive cells were quantified in the sub-mucosal lamina propria region on stained tissues via immunofluorescence. Values are expressed as mean+/−SEM (n=10-12). Non-parametric one-way ANOVA (Kruskal-Wallis) test was used.

FIG. 17A is a graph showing TNF-α cytokine expression. Designer E. coli DSS-induced colitis group shows a general trend in reduction of expression of pro-inflammatory cytokines and increase in expression of anti-inflammatory cytokine. Gene expression of inflammatory cytokines in the colonic tissue of DSS treated probiotic groups performed via qPCR. Values are expressed as mean (n=10-12)+/−SEM.

FIG. 17B is a graph showing IFN-γ cytokine expression. Designer E. coli DSS-induced colitis group shows a general trend in reduction of expression of pro-inflammatory cytokines and increase in expression of anti-inflammatory cytokine. Gene expression of inflammatory cytokines in the colonic tissue of DSS treated probiotic groups performed via qPCR. Values are expressed as mean (n=10-12)+/−SEM.

FIG. 17C is a graph showing IL-1β cytokine expression. Designer E. coli DSS-induced colitis group shows a general trend in reduction of expression of pro-inflammatory cytokines and increase in expression of anti-inflammatory cytokine. Gene expression of inflammatory cytokines in the colonic tissue of DSS treated probiotic groups performed via qPCR. Values are expressed as mean (n=10-12)+/−SEM.

FIG. 17D is a graph showing IL-17a cytokine expression. Designer E. coli DSS-induced colitis group shows a general trend in reduction of expression of pro-inflammatory cytokines and increase in expression of anti-inflammatory cytokine. Gene expression of inflammatory cytokines in the colonic tissue of DSS treated probiotic groups performed via qPCR. Values are expressed as mean (n=10-12)+/−SEM.

FIG. 17E is a graph showing IL-10 cytokine expression. Designer E. coli DSS-induced colitis group shows a general trend in reduction of expression of pro-inflammatory cytokines and increase in expression of anti-inflammatory cytokine. Gene expression of inflammatory cytokines in the colonic tissue of DSS treated probiotic groups performed via qPCR. Values are expressed as mean (n=10-12)+/−SEM.

FIG. 18A is a graph showing TNF-α cytokine expression. Designer L. reuteri DSS-induced colitis group shows a general trend in reduction of expression of pro-inflammatory cytokines and increase in expression of anti-inflammatory cytokine. Gene expression of inflammatory cytokines in the colonic tissue of DSS treated probiotic groups performed via qPCR. Values are expressed as mean (n=10-12)+/−SEM.

FIG. 18B is a graph showing IFN-γ cytokine expression. Designer L. reuteri DSS-induced colitis group shows a general trend in reduction of expression of pro-inflammatory cytokines and increase in expression of anti-inflammatory cytokine. Gene expression of inflammatory cytokines in the colonic tissue of DSS treated probiotic groups performed via qPCR. Values are expressed as mean (n=10-12)+/−SEM

FIG. 18C is a graph showing IL-1p cytokine expression. Designer L. reuteri DSS-induced colitis group shows a general trend in reduction of expression of pro-inflammatory cytokines and increase in expression of anti-inflammatory cytokine. Gene expression of inflammatory cytokines in the colonic tissue of DSS treated probiotic groups performed via qPCR. Values are expressed as mean (n=10-12)+/−SEM

FIG. 18D is a graph showing IL-17a cytokine expression. Designer L. reuteri DSS-induced colitis group shows a general trend in reduction of expression of pro-inflammatory cytokines and increase in expression of anti-inflammatory cytokine. Gene expression of inflammatory cytokines in the colonic tissue of DSS treated probiotic groups performed via qPCR. Values are expressed as mean (n=10-12)+/−SEM.

FIG. 18E is a graph showing IL-10 cytokine expression. Designer L. reuteri DSS-induced colitis group shows a general trend in reduction of expression of pro-inflammatory cytokines and increase in expression of anti-inflammatory cytokine. Gene expression of inflammatory cytokines in the colonic tissue of DSS treated probiotic groups performed via qPCR. Values are expressed as mean (n=10-12)+/−SEM.

FIG. 19 is a graph showing gene expression of Reg3γ in DSS-induced colitis mice. Gene expression of inflammatory cytokines in the colonic tissue of DSS treated L. reuteri probiotic groups performed via qPCR. Values are expressed as mean (n=10-12)+/−SEM. Non-parametric one-way ANOVA (Kruskal-Wallis) test was used.

FIG. 20A is a graph showing gene expression of Reg3γ in DSS-induced colitis mice. Gene expression of inflammatory cytokines in the colonic tissue of DSS treated E. coli probiotic groups performed via qPCR. Values are expressed as mean (n=10-12)+/−SEM. Non-parametric one-way ANOVA (Kruskal-Wallis) test was used.

FIG. 20B is a graph showing gene expression of Muc2 in DSS-induced colitis mice. Gene expression of inflammatory cytokines in the colonic tissue of DSS treated E. coli probiotic groups performed via qPCR. Values are expressed as mean (n=10-12)+/−SEM. Non-parametric one-way ANOVA (Kruskal-Wallis) test was used.

FIG. 21 is a graph showing short chain fatty acid analysis of DSS-induced colitis mice pre-treated with either the E. coli Parent strain, E. coli Designer strain or the DSS control. Short chain fatty acid analysis performed via gas chromatography on cecal samples of mice from each group. Values are expressed as the amount of butyric acid as a weight percentage of the total cecal tissue and shown as mean+/−SEM (n=10-12). Non-parametric one-way ANOVA (Kruskal-Wallis) test was used.

FIG. 22 is a graph showing short chain fatty acid analysis of DSS-induced colitis mice pre-treated with either the L. reuteri Parent strain, L. reuteri Designer strain or the DSS control. Short chain fatty acid analysis performed via gas chromatography on cecal samples of mice from each group. Values are expressed as the amount of butyric acid as a weight percentage of the total cecal tissue and shown as mean+/−SEM (n=10-12). Non-parametric one-way ANOVA (Kruskal-Wallis) test was used.

FIG. 23A is a photograph of the macroscopic examination of cecum and colon from Muc2-deficient mice, showing the Muc2^{-/-} control at 3 months of age at sacrifice. Mice were administered either parent or designer probiotics via oral gavage weekly for 4 weeks. Images were taken to show the colon and ceca of the Muc2^{-/-} colitic mice.

FIG. 23B is a photograph of the macroscopic examination of cecum and colon from Muc2-deficient mice 3 months of age at sacrifice that had been treated with the E. coli parent strain. Mice were administered either parent or designer probiotics via oral gavage weekly for 4 weeks.

FIG. 23C is a photograph of the macroscopic examination of cecum and colon from Muc2-deficient mice at 3 months of age at sacrifice that had been treated with the designer E. coli strain. Mice were administered either parent or designer probiotics via oral gavage weekly for 4 weeks.

FIG. 23D is a photograph of the macroscopic examination of cecum and colon from Muc2-deficient mice, showing the Muc2^{-/-} control at 4 months of age at sacrifice. Mice were administered either parent or designer probiotics via oral gavage weekly for 4 weeks.

FIG. 23E is a photograph of the macroscopic examination of cecum and colon from Muc2-deficient mice at 4 months of age at sacrifice that had been treated with the parent E. coli strain. Mice were administered either parent or designer probiotics via oral gavage weekly for 4 weeks.

FIG. 23F is a photograph of the macroscopic examination of cecum and colon from Muc2-deficient mice at 4 months of age at sacrifice that had treated with the designer E. coli strain. Mice were administered either parent or designer probiotics via oral gavage weekly for 4 weeks.

FIG. 24A is a graph showing weight change in Muc2-deficient mice at 3 months for Muc2^{-/-} control, parent E. coli strain, and designer E. coli strain mice. Weight change calculated as percentage of weight loss from starting body weight. Circles represent E. coli designer strain, squares represent E. coli parent strain, and triangles represent Muc2^{-/-} control. Values are expressed as mean+/−SEM (n=7-11).

FIG. 24B is a graph showing weight change in Muc2-deficient mice at 4 months for Muc2^{-/-} control, parent E. coli strain, and designer E. coli strain mice. Weight change calculated as percentage of weight loss from starting body weight. Circles represent E. coli designer strain, squares represent E. coli parent strain, and triangles represent Muc2^{-/-} control. Values are expressed as mean+/−SEM (n=7-11).

FIG. 25A is a graph showing the clinical scores in Muc2-deficient mice at 3 months that were treated with the parent E. coli strain, the designer E. coli strain or the untreated Muc2^{-/-} control mice. Clinical scores calculated throughout the study are shown. Scores are based on parameters of body movement, rectal bleeding, stool consistency, weight change, and hydration. Circles represent E. coli designer strain, squares represent E. coli parent strain, and triangles represent Muc2^{-/-} control. Values are expressed as mean+/−SEM (n=7-11). Non-parametric one-way ANOVA (Kruskal-Wallis) test was used.

FIG. 25B is a graph showing the clinical scores in Muc2-deficient mice at 4 months that were treated with the parent E. coli strain, the designer E. coli strain or the untreated Muc2^{-/-} control mice. Clinical scores calculated throughout the study are shown. Scores are based on parameters of body movement, rectal bleeding, stool consistency, weight change, and hydration. Circles represent E. coli designer strain, squares represent E. coli parent strain, and triangles represent Muc2^{-/-} control. Values are expressed as mean+/−SEM (n=7-11). Non-parametric one-way ANOVA (Kruskal-Wallis) test was used.

FIG. 26 is a graph showing weight change during duration of colitis in mouse strains, all at 3 months. Weight change calculated as percentage of weight loss from starting body weight. Circles represent mice treated with the E. coli designer strain, squares represent mice treated with the E. coli parent strain, and triangles represent Muc2^{-/-} control mice. Values are expressed as mean+/−SEM (n=15-20).

FIG. 27 is a graph showing clinical scores at 3 months of age of E. coli designer probiotic supplemented Muc2^{-/-} mice, E. coli parent probiotic supplemented Muc2^{-/-} mice or the control Muc2^{-/-} mice. Clinical scores are based on parameters of body movement, rectal bleeding, stool consistency, weight change, and hydration. Circles represent mice treated with the E. coli designer strain, squares represent mice treated with the E. coli parent strain, and triangles represent Muc2^{-/-} control mice. Values are expressed as mean+/−SEM (n=15-20). Non-parametric one-way ANOVA (Kruskal-Wallis) test was used.

FIG. 28A is a graph showing CFU/mL counts at 3 months of age. CFU/ml calculated from homogenates of mesenteric lymph nodes (MLN) grown on 1.8% LB agar. Values are expressed as mean+/−SEM (n=7-12).

FIG. 28B is a graph showing CFU/mL counts at 3 months of age. CFU/ml calculated from homogenates of spleen grown on 1.8% LB agar. Values are expressed as mean+/−SEM (n=7-12).

FIG. 29A is a graph showing CFU/mL counts at 4 months of age. CFU/mL calculated from homogenates of MLN grown on 1.8% LB agar. Values are expressed as mean+/−SEM (n=7-12). Non-parametric t-test (Mann-Whitney U test) was used.

FIG. 29B is a graph showing CFU/mL counts at 4 months of age. CFU/mL calculated from homogenates of spleen grown on 1.8% LB agar. Values are expressed as mean+/−SEM (n=7-12). Non-parametric t-test (Mann-Whitney U test) was used.

FIG. 30A is the amino acid sequence of the N-acetyl-glucosamine binding protein (GbpA) from Vibrio cholerae, UniRef100 accession number: UniRef100_Q9KLD5, SEQ ID NO: 19.

FIG. 30B is the nucleic acid sequence encoding the N-acetyl-glucosamine binding protein (GbpA) from Vibrio cholerae, SEQ ID NO: 26.

FIG. 31A is the amino acid sequence of the N-terminal mucin binding domain (GbpADI) of GbpA from Vibrio cholerae, UniRef100 Accession No: UniRef100_Q9KLD5, SEQ ID NO: 20.

FIG. 31B is a nucleic acid sequence encoding a N-terminal fragment, including the signal peptide, of GbpA from Vibrio cholerae, SEQ ID NO: 27.

FIG. 31C is a harmonized nucleic acid sequence encoding the N-terminal mucin binding domain (GbpADI) of GbpA from Vibrio cholerae (SEQ ID NO: 22)

FIG. 32A is the amino acid sequence of a mucus binding protein (MBP), SEQ ID NO: 21.

FIG. 32B is the amino acid sequence of a mucus binding protein (MBP), SEQ ID NO: 28.

FIG. 32C-1 is a first portion of FIG. 32C, the nucleic acid sequence encoding a mucus binding protein (MBP), SEQ ID NO: 53.

FIG. 32C-2 is a continuation of FIG. 32C-1 and the second portion of FIG. 32C, the nucleic acid sequence encoding a mucus binding protein (MBP), SEQ ID NO: 53.

FIG. 32D is the nucleic acid sequence encoding a mucus binding protein (MBP), SEQ ID NO: 23.

FIG. 33A-1 is a first portion of FIG. 33A, a nucleic acid sequence encoding a GbpA fragment within a MBP nucleotide sequence (SEQ ID NO: 24).

FIG. 33A-2 is a continuation of FIG. 33A-1 and the second portion of FIG. 33A, a nucleic acid sequence encoding a GbpA fragment within a MBP nucleotide sequence (SEQ ID NO: 24).

FIG. 33B is the amino acid sequence of a GbpA-MBP chimeric protein, with the linker sequence indicated in bold and the GbpA fragment underlined, SEQ ID NO: 29.

FIG. 33C-1 is a first portion of FIG. 33C, the nucleic acid sequence encoding a GbpA-MBP chimeric protein, with the linker sequence indicated in bold and the GbpA fragment underlined, SEQ ID NO: 30.

FIG. 33C-2 is a continuation of FIG. 33C-1 and the second portion of FIG. 33C, the nucleic acid sequence encoding a GbpA-MBP chimeric protein, SEQ ID NO: 30.

FIG. 34A-1 is a first portion of FIG. 34A, the nucleic acid sequence of the ttr operon (SEQ ID NO: 25).

FIG. 34A-2 is a continuation of FIG. 34A-1 and the second portion of FIG. 34A, the nucleic acid sequence of the ttr operon (SEQ ID NO: 25).

FIG. 34A-3 is a continuation of FIG. 34A-2 and the third portion of FIG. 34A, the nucleic acid sequence of the ttr operon (SEQ ID NO: 25).

FIG. 34B-1 is a first portion of FIG. 34B, the nucleic acid sequence of the ttrACB operon (SEQ ID NO: 31).

FIG. 34B-2 is a continuation of FIG. 34B-1 and the second portion of FIG. 34B, the nucleic acid sequence of the ttrABC operon (SEQ ID NO: 31).

FIG. 34C is the nucleic acid sequence of the ttrSR operon (SEQ ID NO: 32).

FIG. 34D is the nucleic acid sequence of the ttrA gene (SEQ ID NO: 33).

FIG. 34E is the amino acid sequence of a ttrA protein (SEQ ID NO: 34).

FIG. 34F is the nucleic acid sequence of the ttrB gene (SEQ ID NO: 35).

FIG. 34G is the amino acid sequence of a ttrB protein (SEQ ID NO: 36).

FIG. 34H is the nucleic acid sequence of the ttrC gene (SEQ ID NO: 37).

FIG. 34I is the amino acid sequence of a ttrC protein (SEQ ID NO: 38).

FIG. 34J is the nucleic acid sequence of the ttrR gene (SEQ ID NO: 39).

FIG. 34K is the amino acid sequence of a ttrR protein (SEQ ID NO: 40).

FIG. 34L is the nucleic acid sequence of the ttrS gene (SEQ ID NO: 41).

FIG. 34M is the amino acid sequence of a ttrS protein (SEQ ID NO: 42).

FIG. 35A-1 is a first portion of FIG. 35A, the nucleic acid sequence of the pG+host5 empty vector (SEQ ID NO: 43).

FIG. 35A-2 is a continuation of FIG. 35A-1 and the second portion of FIG. 35A, the nucleic acid sequence of the pG+host5 empty vector (SEQ ID NO: 43).

FIG. 35B-1 is a first portion of FIG. 35B, the nucleic acid sequence of the pG+host5-lar-gbpA vector (SEQ ID NO: 44).

FIG. 35B-2 is a continuation of FIG. 35B-1 and the second portion of FIG. 35B, the nucleic acid sequence of the pG+host5-lar-gbpA vector (SEQ ID NO: 44).

FIG. 36A is the nucleic acid sequence of the pAH162 empty vector (SEQ ID NO: 45).

FIG. 36B-1 is a first portion of FIG. 36B, the nucleic acid sequence of the pAH162-ttrACBSR vector (SEQ ID NO: 46).

FIG. 36B-2 is a continuation of FIG. 36B-1 and the second portion of FIG. 36B, the nucleic acid sequence of the pAH162-ttrACBSR vector (SEQ ID NO: 46).

FIG. 36B-3 is a continuation of FIG. 36B-2 and the third portion of FIG. 36B, the nucleic acid sequence of the pAH162-ttrACBSR vector (SEQ ID NO: 46).

FIG. 37A-1 is the first portion of FIG. 37A, a portion of the nucleic acid sequence of Escherichia coli Nissle 1917 (GenBank Accession No. CP007799.1) with the pAH162-ttrACBSR plasmid integrated to the attB-site of phage phi 80 (the integrated plasmid is underlined) (E. coli Nissle attB^{phi80}::ttrACBSR, (SEQ ID NO: 47).

FIG. 37A-2 is a continuation of FIG. 37A-1 and the second portion of FIG. 37A, a portion of the nucleic acid sequence of Escherichia coli Nissle 1917 (GenBank Accession No. CP007799.1) with the pAH162-ttrACBSR plasmid integrated to the attB-site of phage phi 80 (the integrated plasmid is underlined) (E. coli Nissle attB^{phi80}::ttrACBSR, (SEQ ID NO: 47).

FIG. 37A-3 is a continuation of FIG. 37A-2 and the third portion of FIG. 37A, a portion of the nucleic acid sequence of Escherichia coli Nissle 1917 (GenBank Accession No. CP007799.1) with the pAH162-ttrACBSR plasmid integrated to the attB-site of phage phi 80 (the integrated plasmid is underlined) (E. coli Nissle attB^{phi80}::ttrACBSR, (SEQ ID NO: 47).

FIG. 37B is a portion of the nucleic acid sequence of E. coli Nissle attB^{phi80}::ttrACBSR with the ttrACBSR operon deleted (the integrated plasmid is underlined) (E. coli Nissle attB^{phi80}::Km^{R}, (SEQ ID NO: 48).

FIG. 38A is a photograph of a western blot showing the expression of GbpA domain only in the designer strain.

FIG. 38B is a photograph of a nested PCR test to detect the designer strain using primers for the ttr operon in DNA extracted from the designed strain, or DNA extracted from stool from 12 weeks post-gavage Muc2^{-/-} mice gavaged with the designer strain.

FIG. 38C is a graph showing detection of designer strain expressing GbpA over time in colon samples from colitic Muc2^{-/-} and healthy C57BL/6 mice.

FIG. 38D is a graph showing is a graph showing detection of designer strain expressing ttr operon over time in stool samples from Muc2^{-/-} and C57BL/6 mice.

FIG. 39A is a graph showing weight loss during DSS treatment period for mice. Circles represent the group of mice treated with the recombinant probiotic strain, labelled as the BioPersist (E. coli Nissle attB^{phi80}::ttrACBSR). Squares represent the group of mice treated with 5-ASA. Triangles represent the no treatment DSS control, labelled as Vehicle. Weight loss was calculated as percentage of weight loss from starting body weight prior to DSS exposure. Values are expressed as mean+/−SD (n=10).

FIG. 39B is a graph showing clinical scores following DSS-induced colitis in mice. Circles represent the group of mice treated with the recombinant probiotic strain, labelled as BioPersist (E. coli Nissle attB^{phi80}::ttrACBSR). Squares represent the group of mice treated with 5-ASA. Triangles represent the no treatment DSS control, labelled as Vehicle. Movement, rectal bleeding, stool consistency, weight loss, and hydration were used to calculate clinical scores. Values expressed as mean+/−SD (n=10).

FIG. 39C is a graph and photograph showing colon length upon tissue collection. Circles represent the group of mice treated with the recombinant probiotic strain, labelled as BioPersist (E. coli Nissle attB^{phi80}::ttrACBSR). Squares represent the group of mice treated with 5-ASA. Triangles represent the no treatment DSS control.

FIG. 39D is a fluorescence in situ hybridization image of colon sections of mice treated with the recombinant probiotic strain upon tissue collection.

FIG. 39E is a graph showing the total histopathological scores of the DSS control (labeled as Vehicle), 5-ASA, and E. coli designer strain (labeled as BioPersist). H&E stained slides of cross sections of the distal colon were used to calculate histopathological scores. Epithelial integrity, immune cell infiltration, ulceration, and goblet cell depletion were used to calculate histopathological scores in cross sections of the distal colon. Values are expressed as mean+/−SD (n=10). Non-parametric one-way ANOVA (Kruskal-Wallis) test was used.

FIG. 39F is a graph showing a comparison of TNF-α cytokine expression between the DSS control (labeled as Vehicle), 5-ASA, and E. coli designer strain (labeled as BioPersist). Gene expression of inflammatory cytokines in the colonic tissue of DSS treated probiotic groups performed via qPCR. Values are expressed as mean (n=10)+/−SD.

FIG. 39G is a graph showing a comparison of INF-γ cytokine expression between the DSS control (labeled as Vehicle), 5-ASA, and E. coli designer strain (labeled as BioPersist). Gene expression of inflammatory cytokines in the colonic tissue of DSS treated probiotic groups performed via qPCR. Values are expressed as mean (n=10)+/−SD.

FIG. 39H is a graph showing a comparison of IL-17A cytokine expression between the DSS control (labeled as Vehicle), 5-ASA, and E. coli designer strain (labeled as BioPersist). Gene expression of inflammatory cytokines in the colonic tissue of DSS treated probiotic groups performed via qPCR. Values are expressed as mean (n=10)+/−SD.

FIG. 39I is a graph showing a comparison of IL-10 cytokine expression between the DSS control (labeled as Vehicle), 5-ASA, and E. coli designer strain (labeled as BioPersist). Gene expression of cytokines in the colonic tissue of DSS treated probiotic groups performed via qPCR. Values are expressed as mean (n=10)+/−SD.

FIG. 39J is a graph showing a comparison of IL-22 cytokine expression between the DSS control (labeled as Vehicle), 5-ASA, and E. coli designer strain (labeled as BioPersist). Gene expression of cytokines in the colonic tissue of DSS treated probiotic groups performed via qPCR. Values are expressed as mean (n=10)+/−SD.

FIG. 39K is a graph showing a comparison of Reg3-γ expression between the DSS control (labeled as Vehicle), 5-ASA, and E. coli designer strain (labeled as BioPersist). Gene expression of inflammatory cytokines in the colonic tissue of DSS treated probiotic groups performed via qPCR. Values are expressed as mean (n=10)+/−SD.

FIG. 39L is a graph showing a comparison of macrophage count between the DSS control (labeled as Vehicle), 5-ASA, and E. coli designer strain (labeled as BioPersist). Values are expressed as mean (n=10)+/−SD.

FIG. 39M is a graph showing a comparison of neutrophil count between the DSS control (labeled as Vehicle), 5-ASA, and E. coli designer strain (labeled as BioPersist). Values are expressed as mean (n=10)+/−SD.

### DETAILED DESCRIPTION

The present disclosure relates, in part, to probiotic compositions and uses thereof. In some embodiments, a probiotic composition in accordance with the present disclosure may exhibit increased colonization and persistence in the gastrointestinal tract of a subject. In some embodiments, a probiotic composition in accordance with the present disclosure may prevent, reduce or ameliorate inflammation in the gastrointestinal tract of a subject.

The gastrointestinal tract or “GI” tract is often the site of inflammation. Inflammation of the GI tract has been correlated to several disorders including, but not limited to, ulcers, gastritis, inflammatory bowel disease, etc. The terms “inflammatory bowel disease” (IBD), “irritable bowel disease”, “irritable bowel syndrome”, or “intestinal inflammation,” as used herein, refer to or describe a group of physiological conditions that are typically associated with intestinal inflammation, abdominal pain, cramping, constipation or diarrhea. IBD includes ulcerative colitis and Crohn's disease.

The term “probiotic bacteria” refers to live bacteria, which may confer health benefits to their host when administered in sufficient amounts. Probiotic bacteria may be useful in the prophylaxis and/or treatment of undesirable inflammatory activity, especially undesirable gastrointestinal inflammatory activity, such as inflammatory bowel disease, irritable bowel syndrome, or intestinal inflammation. In some embodiments, a probiotic bacterium, as used herein, may be any probiotic bacterium amenable to recombinant techniques. Examples of probiotic bacteria include, but are not limited to, specific probiotic strains of Lactobacillus, Bifidobacterium, Lactococcus, Enterococcus, Streptococcus, Pediococcus, Leuconostoc, Bacillus, or Escherichia coli. In some embodiments, a probiotic Lactobacillus may include, without limitation, a Lactobacillus reuteri, Lactobacillus plantarum, Lactobacillus casei (such as Lactobacillus casei Shirota), Lactobacillus salivarius, Lactobacillus paracasei, Lactobacillus lactis, Lactobacillus acidophilus, Lactobacillus sakei, Lactobacillus brevis, Lactobacillus buchneri, Lactobacillus fermentum, Lactobacillus delbrueckii, Lactobacillus delbrueckii subsp. bulgaricus, Lactobacillus helveticus, Lactobacillus garvieae, Lactobacillus acetotolerans, Lactobacillus agilis, Lactobacillus algidus, Lactobacillus alimentarius, Lactobacillus amylolyticus, Lactobacillus amylophilus, Lactobacillus amylovorus, Lactobacillus animalis, Lactobacillus aviarus, Lactobacillus bifermentans, Lactobacillus bulgaricus, Lactobacillus carnis, Lactobacillus caternaformis, Lactobacillus cellobiosis, Lactobacillus collinoides, Lactobacillus confuses, Lactobacillus coryniformis, Lactobacillus crispatus, Lactobacillus curvatus, Lactobacillus divergens, Lactobacillus farciminis, Lactobacillus fructivorans, Lactobacillus fructosus, Lactobacillus gallinarum, Lactobacillus gasseri, Lactobacillus graminis, Lactobacillus halotolerans, Lactobacillus hamster, Lactobacillus heterohiochii, Lactobacillus hilgardii, Lactobacillus homohiochii, Lactobacillus iners, Lactobacillus intestinalis, Lactobacillus jensenii, Lactobacillus johnsonii, Lactobacillus kandleri, Lactobacillus kefiri, Lactobacillus kefuranofaciens, Lactobacillus kefirgranum, Lactobacillus kunkeei, Lactobacillus leichmannii, Lactobacillus lindneri, Lactobacillus malefermentans, Lactobacillus mali, Lactobacillus maltaromicus, Lactobacillus manihotivorans, Lactobacillus minor, Lactobacillus minutus, Lactobacillus mucosae, Lactobacillus murinus, Lactobacillus nagelii, Lactobacillus oris, Lactobacillus panis, Lactobacillus parabuchneri, Lactobacillus paracasei, Lactobacillus parakefiri, Lactobacillus paralimentarius, Lactobacillus paraplantarum, Lactobacillus pentosus, Lactobacillus perolens, Lactobacillus piscicola, Lactobacillus plantarum, Lactobacillus pontis, Lactobacillus rhamnosus, Lactobacillus rhamnosus GG, Lactobacillus rimae, Lactobacillus rogosae, Lactobacillus ruminis, Lactobacillus sanfranciscensis, Lactobacillus sharpeae, Lactobacillus suebicus, Lactobacillus trichodes, Lactobacillus uli, Lactobacillus vaccinostercus, Lactobacillus vaginalis, Lactobacillus viridescens, Lactobacillus vitulinus, Lactobacillus xylosus, Lactobacillus yamanashiensis, or a Lactobacillus zeae. In some embodiments, a probiotic Escherichia coli may be E. coli Nissle 1917 (complete genome set forth in Accession No. CP007799.1; www[dot]ncbi[dot]nlm[dot]nih[dot]gov/nuccore/CP007799.1?report=fasta) or a subspecies or strain thereof. In some embodiments, a probiotic Bifidobacterium may be Bifidobacterium infantis, Bifidobacterium adolescentis, Bifidobacterium animalis subsp animalis, Bifidobacterium longum, Bifidobacterium fidobacterium breve, Bifidobacterium bifidum, Bifidobacterium animalis subsp. lactis or Bifidobacterium lactis, such as Bifidobacterium lactis DN-173 010. In some embodiments, a probiotic Bacillus may be Bacillus coagulans. In some embodiments, a probiotic Lactococcus may be Lactococcus lactis subsp. Lactis such as Lactococcus lactis subsp. lactis CV56. In some embodiments, a probiotic Enterococcus may be Enterococcus durans. In some embodiments, a probiotic Streptococcus may be Streptococcus thermophilus. In some embodiments, the probiotic bacterium may be an auxotrophic strain designed, for example, to limit its survival outside of the human or animal intestine, using standard techniques.

The term “recombinant” means that something has been recombined, so that when made in reference to a nucleic acid construct the term refers to a molecule that is comprised of nucleic acid sequences that are joined together or produced by means of molecular biological techniques. The term “recombinant” when made in reference to a protein or a polypeptide refers to a protein or polypeptide molecule which is expressed using a recombinant nucleic acid construct created by means of molecular biological techniques. The term “recombinant” when made in reference to genetic composition refers to a gamete or progeny with new combinations of alleles that did not occur in the parental genomes. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as ‘recombinant’ therefore indicates that the nucleic acid molecule has been manipulated using genetic engineering, i.e. by human intervention. Recombinant nucleic acid constructs may for example be introduced into a host cell, such as a probiotic bacterium, to generate a “recombinant probiotic bacterium.” Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events, including the use of integrative vectors, site specific recombination or CRISPR-mediated engineering.

The term “GbpA,” as used herein, refers to a N-acetyl glucosamine binding protein A. In some embodiments, a suitable GbpA protein, or homologue thereof, may be isolated from a pathogenic bacterium. In some embodiments, a suitable GbpA protein, or homologue thereof, may be isolated from a bacterial species from the phyla Gammaproteobacteria, Enterobacteria or Firmicutes. In some embodiments, a suitable GbpA protein, or homologue thereof, may be isolated from a bacterium including, but not limited to, Vibrio spp, Escherichia ssp., Yersinia ssp., Shewanella ssp., Photobacterium ssp., Listeria ssp., Enterobacter ssp., Aeromonas ssp., Klebsiella ssp. or Aliivibrio ssp. In some embodiments, a GbpA protein, or homologue thereof, may be isolated from Vibrio spp, including, but not limited to, V. cholerae, V. mimicus, V. metoecus, V. vulnificus, V. parahaemolyticus, or V. fischeri. In some embodiments, a GbpA protein, or homologue thereof, may be isolated from Yersinia spp, including, but not limited to, Yersinia enterocolitica. In some embodiments, a homologue of a GbpA protein may include, without limitation, a sequence as set forth in Accession Nos. YP_001007736.1, WP_057644048.1, WP_049605074.1, WP_053010295.1, WP_050077216.1, AUD62036.1, OXS01804.1, KPN78673.1, KEK29442.1, AAN54144.1, OUM13866.1, WP_011220398.1, OCH04476.1, WP_083198965.1, WP_081091566.1, WP_049940440.1, WP_065604524.1, KRT36821.1, WP_032608383, WP_015455208.1, OUY95058.1, PJI14410.1, OXV29379.1, PJZ14491.1, ATP91661.1, ATY82669.1, OSP53097.1, WP_102803702.1, OLP12672.1, or PDO74205.

In some embodiments, a GbpA protein may have the amino acid sequence set forth in NCBI Accession No. KKP14471. In some embodiments, a GbpA protein may have a sequence substantially identical to the amino acid sequence set forth in SEQ ID NO: 19, for example, at least 30%, 35%, 40%, 45%, 50%, 55%, 60%, 65%, 70%, 75%, 80%, 85%, 90%, 95% or 99% amino acid sequence identity to the amino acid sequence set forth in SEQ ID NO: 19. In some embodiments, a GbpA protein may be encoded by the nucleic acid sequence set forth in SEQ ID NO: 26. In some embodiments, a GbpA protein may include a mucin binding domain, referred to as “GbpADI,” from a GbpA protein from Vibrio cholerae.

In alternative embodiments, a GbpA protein may include the full-length protein as well as fragments, isoforms or homologue thereof. In some embodiments, a fragment of a GbpA protein may be a non-pathogenic fragment. In some embodiments, a fragment of a GbpA protein may include a fragment including the mucin binding domain or a portion thereof, as long as mucin binding activity is retained. In some embodiments, a fragment of a GbpA protein may include an amino acid sequence substantially identical to SEQ ID NO: 20, for example, at least 30%, 35%, 40%, 45%, 50%, 55%, 60%, 65%, 70%, 75%, 80%, 85%, 90%, 95% or 99% amino acid sequence identity to the amino acid sequence set forth in SEQ ID NO: 20. In some embodiments, a fragment of a GbpA protein may be encoded by the nucleic acid sequence set forth in SEQ ID NO: 27 or a portion thereof.

In alternative embodiments, a GbpA protein may be harmonized, for example, for expression in a particular host. In some embodiments, a harmonized GbpA protein may include a sequence harmonized for expression in L. reuterii, for example, as set forth in SEQ ID NO: 22, or a sequence having substantial identity thereto, for example, at least 30%, 35%, 40%, 45%, 50%, 55%, 60%, 65%, 70%, 75%, 80%, 85%, 90%, 95% or 99% sequence identity to the nucleic acid sequence set forth in SEQ ID NO: 22.

In alternative embodiments, a GbpA protein may include a form that results from processing within a cell, such as truncated forms.

A “bacterial surface protein,” as used herein, refers to a protein associated with, or protruding from, the cell wall of a bacterium. Accordingly, in some embodiments, a bacterial surface protein may be anchored to, or embedded in and protruding from, the cell wall of a bacterium or may be associated with such a protein. In some embodiments, a bacterial surface protein may be a mucin binding protein, an S-layer protein (for example, a Lactobacillus S-layer protein), an integrin, a G-coupled protein, a mannose-binding lectin (for example, a Lactobacillus mannose-binding lectin), fimbria or flagella (for example, from E. coli) or any surface projection that may bind with host mucosae. In some embodiments, a bacterial surface protein may include, without limitation, a S-layer protein, such as slpA of Lactobacillus acidophilus, UniProt Accession No. P35829 or CbsA of Lactobacillus crispatus, UniProt Accession No. 007120; an integrin-binding protein, such as collagen-binding protein cnb Lactobacillus reuteri, UniProt Accession No. E21Q97; a fimbria, such fimA of E. coli, UniProt Accession No. Q1 R2K0); or a mucus binding protein, such as from Lactobacillus acidophilus UniProt Accession No. Q5FJA7.

The term “MBP,” as used here, refers to a bacterial surface protein known as “mucus binding protein.” The MBP protein may be isolated from various bacteria, including non-pathogenic bacteria including, but not limited to, Lactobacillus. In some embodiments, an MBP protein may be isolated from a probiotic bacterium. In some embodiments, an MBP protein may be isolated from Lactobacillus reuteri.

In some embodiments, an MBP protein may be the “hypothetical protein LAR_0958” of Lactobacillus reuteri JCM 1112. In some embodiments, an MBP protein may have the amino acid sequence set forth in NCBI Accession No. BAG25474.1. In some embodiments, a MBP protein may have an amino acid sequence substantially identical to SEQ ID NO: 21 or SEQ ID NO: 28, for example, at least 30%, 35%, 40%, 45%, 50%, 55%, 60%, 65%, 70%, 75%, 80%, 85%, 90%, 95% or 99% amino acid sequence identity to the amino acid sequence set forth in SEQ ID NO: 21 or SEQ ID NO: 28. In some embodiments, a MBP protein may encompass the full-length protein, as well as isoforms, fragments or homologues thereof. In alternative embodiments, a MBP protein includes a form that results from processing within a cell. In some embodiments, a MBP protein may be encoded by the nucleic acid sequence substantially identical to SEQ ID NO: 23 or SEQ ID NO: 53 or a fragment thereof.

In some embodiments, a GbpA protein or fragment thereof may be co-expressed, for example, as part of a surface protein operon, or recombined with a bacterial surface protein. In some embodiments, multiple copies of a GbpA protein or fragment thereof may be expressed in combination with a repeating surface protein, such as fimbriae. In embodiments where a GbpA protein or fragment thereof is recombined with a bacterial surface protein, it is to be understood that the exact location of the GbpA protein within the bacterial surface protein is not important, as long as the recombined GbpA protein or fragment thereof is expressed on the surface of a host cell, such as a probiotic bacterium, and can bind to an organic surface, such as an intestinal cell surface or a mucin.

In some embodiments, a GbpA protein or fragment thereof may be recombined with a MBP protein or fragment thereof to form a chimeric GbpA-MBP protein. In some embodiments, the GbpA protein fragment may be the mucin binding domain, or a mucin binding portion thereof.

In some embodiments, a chimeric GbpA-MBP protein may have a sequence substantially identical to SEQ ID NO: 29, for example, at least 30%, 35%, 40%, 45%, 50%, 55%, 60%, 65%, 70%, 75%, 80%, 85%, 90%, 95% or 99% amino acid sequence identity to the amino acid sequence set forth in SEQ ID NO: 29. In some embodiments, a chimeric GbpA-MBP protein may be encoded by a nucleic acid sequence substantially identical to SEQ ID NO: 24 or SEQ ID NO: 30.

A “substantially identical” sequence is an amino acid or nucleotide sequence that differs from a reference sequence only by one or more conservative substitutions, as discussed herein, or by one or more non-conservative substitutions, deletions, or insertions located at positions of the sequence that do not destroy the biological function of the amino acid or nucleic acid molecule. Such a sequence can be any value from 30% to 99%, or more generally at least 30%, 40%, 50, 55% or 60%, or at least 65%, 75%, 80%, 85%, 90%, or 95%, or as much as 96%, 97%, 98%, or 99% identical when optimally aligned at the amino acid or nucleotide level to the sequence used for comparison using, for example, the Align Program (Myers and Miller, CABIOS, 1989, 4:11-17) or FASTA. For polypeptides, the length of comparison sequences may be at least 2, 5, 10, or 15 amino acids, or at least 20, 25, or 30 amino acids. In alternate embodiments, the length of comparison sequences may be at least 35, 40, or 50 amino acids, or over 60, 80, or 100 amino acids. For nucleic acid molecules, the length of comparison sequences may be at least 5, 10, 15, 20, or 25 nucleotides, or at least 30, 40, or 50 nucleotides. In alternate embodiments, the length of comparison sequences may be at least 60, 70, 80, or 90 nucleotides, or over 100, 200, or 500 nucleotides. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine, or as described herein). Examples of useful software include the programs Pile-up and PrettyBox. Such software matches similar sequences by assigning degrees of homology to various substitutions, deletions, substitutions, and other modifications.

Alternatively, or additionally, two nucleic acid sequences may be “substantially identical” if they hybridize under high stringency conditions. In some embodiments, high stringency conditions are, for example, conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. (These are typical conditions for high stringency northern or Southern hybridizations.) Hybridizations may be carried out over a period of about 20 to 30 minutes, or about 2 to 6 hours, or about 10 to 15 hours, or over 24 hours or more. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998.

In some embodiments, a chimeric GbpA-bacterial surface protein, such as a chimeric GbpA-MBP protein, may include flexible linkers between the GbpA and bacterial surface protein components to, for example, facilitate presentation of the GbpA moiety. It is to be understood that the linker may be of any length or composition, as long as the linker facilitates presentation of the GbpA moiety on the bacterial surface. In some embodiments, the linkers may be about 10 to about 30 amino acids in length, for example, 10, 11, 12, 13, 14, 15, 16, 17, 18, 19, 20, 21, 22, 23, 24, 25, 26, 27, 28, 29, or 30 amino acids in length. In alternative embodiments, the linker may be longer or shorter. In some embodiments, the linkers may have the amino acid sequence:

(SEQ ID NO: 49)
GSAGSAEAGSNWSHPQFEKGSAGSAAGS
or
(SEQ ID NO: 50)
GSAGSAAGSGEF,

although it is to be understood that any suitable linker sequence may be used.

In some embodiments, the linkers may have the nucleic acid sequence:

(SEQ ID NO: 51)
ggtagtgctggtagtgctgaagctggtagtaattggagtcatccacaa
tttgaaaaaggtagtgctggtagtgctgctggtagt
or
(SEQ ID NO: 52)
ggtagtgctggtagtgctgctggtagtggtgaattt,

although it is to be understood that any suitable linker sequence may be used.

The term “ttr,” as used herein, refers to tetrathionate reductase, which is involved in making tetrathionate available as an electron acceptor through the reduction of tetrathionate to thiosulfate.

In some embodiments, genes encoding tetrathionate reductase include the ttrACBSR operon from Salmonella enterica; the ttrA, ttrC, ttrB, ttrR and ttrS genes from Salmonella enterica; the ttrA, ttrC, and ttrB genes from Salmonella enterica, or a homologue, isoform or fragment thereof. In some embodiments, a ttr protein or operon may be isolated from a bacterium of the Enterobacteriaceae family, such as a Salmonella ssp., Yersinia ssp., Proteus ssp., Citrobacter ssp., Klebsiella sp., Raoultella sp., Escherichia sp., Serratia sp., Leclercia sp., Morganella sp., Providencia sp. or Enterobacter sp., or of the Vibrionaceae family, such as a Vibrio ssp. In some embodiments, a ttr protein or operon may be isolated from a Yersinia enterocolitica, Proteus mirabilis, Escherichia coli, Serratia marcescens, Leclerica adecarboxylata, Morganella morganii, Citrobacter freundii, Klebsiella oxytoca, Raoultella ornithinolytica, Vibrio cyclitrophicus, Providencia alcalifaciens PAL3, or Enterobacter sp GN02600. In some embodiments, a ttr protein or operon may be isolated from Salmonella enterica subsp. enterica (ex Kauffmann and Edwards) Le Minor and Popoff serovar Typhimurium (ATCC® 14028^{T}™). In some embodiments, a homologue of a tetrationate reducatase may include, without limitation, a molybdopterin oxidoreductase, an octaheme tetrathionate reductase or a bifunctional thiosulfate dehydrogenase/tetrathionate reductase.

In some embodiments, a ttrA protein may include, without limitation, an amino acid sequence as set forth in Accession No. NP_460348 or SEQ ID NO: 34. In some embodiments, a ttrA protein may be encoded by, without limitation, a nucleic acid sequence as set forth in SEQ ID NO: 33. In some embodiments, a ttrA protein may include, without limitation, an amino acid sequence, or be encoded by a nucleic acid sequence, having at least about 36% identity thereto, for example, at least 36%, 40%, 45%, 50%, 55%, 60%, 65%, 70%, 75%, 80%, 85%, 90%, 95% or 99% sequence identity to the sequence set forth in SEQ ID NO: 34 or SEQ ID NO: 33, respectively. In some embodiments, a homologue of a ttrA protein may include, without limitation, a molybdopterin oxidoreductase. In some embodiments, a homologue of a ttrA protein may include, without limitation, a sequence as set forth in GenBank Accession Nos. YP_001005907.1, EEQ20500.1, EEQ14547.1, AKP35086.1, KSW19446.1, OZS67160.1, KZE53847.1, WP_036976853.1, KPR51726.1, WP_044699957.1, AKE58784.1, CEJ67217.1, KHE12612.1, SBL10805.1, OVJ00655.1, AJF72717.1, OMP97259.1, KXQ61755.1, KPO10992.1, KXP28341.1, ALE97083.1, KFF88851.1, ALX93812.1, AKE11813.1, ALZ97153.1, AGG30792.1, WP_067426732.1, or KLQ21159.1.

In some embodiments, a ttrB protein may include, without limitation, an amino acid sequence as set forth in Accession No. NP_460350 or SEQ ID NO: 36. In some embodiments, a ttrB protein may be encoded by, without limitation, a nucleic acid sequence as set forth in SEQ ID NO: 35. In some embodiments, a ttrB protein may include, without limitation, an amino acid sequence, or be encoded by a nucleic acid sequence, having at least about 37% identity thereto, for example, at least 37%, 40%, 45%, 50%, 55%, 60%, 65%, 70%, 75%, 80%, 85%, 90%, 95% or 99% sequence identity to the sequence set forth in SEQ ID NO: 36 or SEQ ID NO: 35, respectively. In some embodiments, a homologue of a ttrB protein may include, without limitation, a 4Fe-4S ferredoxin, for example, from Vibrio cyclitrophicus. In some embodiments, a homologue of a ttrB protein may include, without limitation, a sequence as set forth in GenBank Accession Nos. YP_001005905.1, CFQ93022.1, CFQ43076.1, CRY54230.1, CAR43509.1, WP_036971149.1, AVA40532.1, GAL39716.1, GAL44236.1, PKQ50411.1, AMG54481.1, WP_103814386.1, PPA47719.1, WP_094310326.1, WP_041145060 WP_076945285.1 WP_077910396.1 WP_085949444.1, WP_060452523.1, SMZ55374.1, SMB25440.1, AMG99006.1, WP_059308319.1, WP_024473892.1, WP_067402438.1 or WP_019076686.1.

In some embodiments, a ttrC protein may include, without limitation, an amino acid sequence as set forth in Accession No. NP_460349 or SEQ ID NO: 38. In some embodiments, a ttrC protein may be encoded by, without limitation, a nucleic acid sequence as set forth in SEQ ID NO: 37. In some embodiments, a ttrC protein may include, without limitation, an amino acid sequence, or be encoded by a nucleic acid sequence, having at least about 39% identity thereto, for example, at least 39%, 40%, 45%, 50%, 55%, 60%, 65%, 70%, 75%, 80%, 85%, 90%, 95% or 99% sequence identity to the sequence set forth in SEQ ID NO: 38 or SEQ ID NO: 37, respectively. In some embodiments, a homologue of a ttrC protein may include, without limitation, a polysulfide reductase NrfD, for example, from a Providencia alcalifaciens PAL-3. In some embodiments, a homologue of a ttrC protein may include, without limitation, a sequence as set forth in GenBank Accession Nos. WP_077173918.1, WP_057615346.1, WP_057646861.1, WP_012368068.1, WP_087802132.1, WP_086551155.1, PKQ50348.1, WP_096757206.1, WP_080858725.1, WP_085521140.1, WP_102802900.1, WP_041145059.1, WP_076945284.1, WP_044864557.1, WP_094461085.1, WP_047730217.1, WP_059308318.1, WP_004236882.1, WP_067426730.1, or WP_047358863.1.

In some embodiments, a ttrR protein may include, without limitation, an amino acid sequence as set forth in Accession No. NP_460352 or SEQ ID NO: 40. In some embodiments, a ttrR protein may be encoded by, without limitation, a nucleic acid sequence as set forth in SEQ ID NO: 39. In some embodiments, a ttrR protein may include, without limitation, an amino acid sequence, or be encoded by a nucleic acid sequence, having at least about 43% identity thereto, for example, at least 43%, 45%, 50%, 55%, 60%, 65%, 70%, 75%, 80%, 85%, 90%, 95% or 99% sequence identity to the sequence set forth in SEQ ID NO: 40 or SEQ ID NO: 39, respectively. In some embodiments, a homologue of a ttrR protein may include, without limitation, a DNA-binding response regulator for example from Escherichia coli. In some embodiments, a homologue of a ttrR protein may include, without limitation, a sequence as set forth in GenBank Accession Nos. YP_001005903.1, CRL60521.1, KKJ88792.1, OUE56241.1, AID90294.1, AIE70476.1, AJF75264.1, KPO10996.1, SAY44133.1, KJY05630.1 or KLQ21155.1.

In some embodiments, a ttrS protein may include, without limitation, an amino acid sequence as set forth in Accession No. NP_460351 or SEQ ID NO: 42. In some embodiments, a ttrS protein may be encoded by, without limitation, a nucleic acid sequence as set forth in SEQ ID NO: 41. In some embodiments, a ttrS protein may include, without limitation, an amino acid sequence, or be encoded by a nucleic acid sequence, having at least about 36% identity thereto, for example, at least 36%, 40%, 45%, 50%, 55%, 60%, 65%, 70%, 75%, 80%, 85%, 90%, 95% or 99% sequence identity to the sequence set forth in SEQ ID NO: 42 or SEQ ID NO: 41, respectively. In some embodiments, a homologue of a ttrS protein may include, without limitation, a sensor histidine kinase from Enterobacteriacea. In some embodiments, a homologue of a ttrS protein may include, without limitation, a sequence as set forth in GenBank Accession Nos. YP_001005904.1, CFR17843.1, CNE64519.1, CAR43511.1, EST58419.1 or ALE97086.1.

In some embodiments, the tetrathionate respiratory operon includes the nucleic acid sequence set forth in SEQ ID NO: 25 or a sequence having at least about 40% identity thereto for example, at least 40%, 45%, 50%, 55%, 60%, 65%, 70%, 75%, 80%, 85%, 90%, 95% or 99% sequence identity to the nucleic acid sequence set forth in SEQ ID NO: 25. In some embodiments, the tetrathionate respiratory operon includes the nucleic acid sequence set forth in SEQ ID NO: 31 (ttrACB operon) or a sequence having at least about 40% identity thereto for example, at least 40%, 45%, 50%, 55%, 60%, 65%, 70%, 75%, 80%, 85%, 90%, 95% or 99% sequence identity to the nucleic acid sequence set forth in SEQ ID NO: 31. In some embodiments, the tetrathionate respiratory operon may additionally include the nucleic acid sequence set forth in SEQ ID NO: 32 (ttrSR operon) or a sequence having at least about 40% identity thereto for example, at least 40%, 45%, 50%, 55%, 60%, 65%, 70%, 75%, 80%, 85%, 90%, 95% or 99% sequence identity to the nucleic acid sequence set forth in SEQ ID NO: 32.

A “vector” is a DNA molecule derived, for example, from a plasmid or bacteriophage, into which a nucleic acid molecule, for example, encoding a GbpA protein, a bacterial surface protein or a tetrathionate reductase, or a fragment thereof, may be inserted. A vector may contain one or more unique restriction sites and may be capable of autonomous replication in a defined host or vehicle organism such that the cloned sequence is reproducible. A vector may be a DNA expression vector, i.e, any autonomous element capable of directing the synthesis of a recombinant polypeptide, and thus may be used to express a polypeptide, for example a GbpA protein, a bacterial surface protein or a tetrathionate reductase, or a fragment thereof, in a host cell. DNA expression vectors include bacterial plasmids and phages and mammalian and insect plasmids and viruses. In some embodiments, a vector may integrate into the genome of the host cell, such that any modification introduced into the genome of the host cell by the vector becomes part of the genome of the host cell. In some embodiments, a vector may remain as an autonomously replicating unit, such as a plasmid. Accordingly, the term “expression vector,” as used herein, refers to a polynucleotide composition which may be integrating or autonomous, (i.e. self-replicating), and which contains the necessary components to achieve transcription of an expressible sequence in a target cell, when introduced into the target cell. Expression vectors may include plasmids, cosmids, bacterial artificial chromosomes (BACs), viruses, etc. An expression vector optionally contains nucleic acid elements that facilitate replication of the vector, elements that facilitate integration of the vector into the genome of the target host cell, elements which confer properties, for example antibiotic resistance, to the target host cell which allow selection or screening of transformed cells and the like. Techniques and methods for design and construction of expression vectors are described herein and well known in the art.

A vector in accordance with the present disclosure may be used to express a GbpA protein, a bacterial surface protein and/or a tetrathionate reductase, or a fragment thereof, in a prokaryotic host cell, such as a probiotic bacterium.

In some embodiments, a GbpA protein or fragment thereof may be expressed on the surface of a probiotic bacterium. In some embodiments, a GbpA protein or fragment thereof may be expressed on the surface of a probiotic bacterium such that it can bind to an organic surface, such as an intestinal cell surface or a mucin. In some embodiments, the GbpA protein or fragment thereof may be expressed on the surface of a probiotic bacterium as part of a bacterial surface protein as a single repeat or in multiple repeats. In alternative embodiments, the GbpA protein or fragment thereof may be expressed on the surface of a probiotic bacterium as a single, separate protein including a membrane-anchoring sequence and/or signal peptide, and may be integrated into the bacterial chromosome. In alternative embodiments, the GbpA protein or fragment thereof may be expressed on the surface of a probiotic bacterium as part of a complex, multi-domain protein, each domain of which includes a GbpA binding domain, and may be integrated into the bacterial chromosome; the multi-domain protein may include a membrane-anchoring sequence and/or signal peptide. Membrane-anchoring sequences are known in the art and may include, without limitation, a LXPTG-motif cell wall, S-layer homology (SLH) domains, lipoproteins, amino-terminal membrane anchors or transmembrane domains. Signal peptides are known in the art and may include, without limitation, a YSIRK-G/S motif signal peptide or exemplary signal peptides as described in Ivankov D N, Payne S H, Galperin M Y, Bonissone S, Pevzner P A, Frishman D. How many signal peptides are there in bacteria? Environmental microbiology. 2013; 15(4):983-990 or Payne S H, Bonissone S, Wu S, Brown R N, Ivankov D N, Frishman D, Pasa-Tolic L, Smith R D, Pevzner P A. Unexpected diversity of signal peptides in prokaryotes. MBio. 2012; 3(6). Pii: e00339-12.

In some embodiments, a tetrathionate reductase, or a fragment thereof, may be expressed in a gram-negative bacterium. In some embodiments, a tetrathionate reductase, or a fragment thereof, may be expressed in a probiotic Escherichia coli, such as E. coli Nissle 1917 (complete genome set forth in Accession No. CP007799.1; www[dot]ncbi[dot]nlm[dot]nih[dot]gov/nuccore/CP007799.1?report=fasta) or a subspecies or strain thereof. In some embodiments, a tetrathionate reductase, or a fragment thereof, may be may be integrated into the bacterial chromosome. In some embodiments, a tetrathionate reductase may be expressed by expression of the ttrA, ttrB and ttrC genes separately, in combination with an oxygen-sensitive promoter-operator that, for example, includes a binding site for an oxygen-responding transcription factor such as the fumarate-nitrate reduction regulator (FNR) transcription factor or the aerobic respiration control (ArcA) transcription factor. In some embodiments, an oxygen-sensitive promoter-operator may include, without limitation, a fumarate-nitrate reduction regulator (FNR) transcription factor, aerobic respiration control (ArcA) transcription factor, FixL-FixJ system of Sinorhizobium meliloti, DosT/DevS system found in Mycobacterium tuberculosis, nar operon of Escherichia coli, vgb operon of Vitreoscilla hemoglobin, arc operon of Staphylococcus aureus, etc. In some embodiments, a tetrathionate reductase may be expressed by an operon including the ttrA, ttrB and ttrC genes, in combination with an oxygen-sensitive promoter-operator. In some embodiments, a tetrathionate reductase may be expressed by expression of the ttrA, ttrB, ttrC, ttrR and ttrS genes separately. In some embodiments, a tetrathionate reductase may be expressed by an operon including the ttrA, ttrB, ttrC, ttrR and ttrS genes.

In some embodiments, nucleic acid sequences encoding a GbpA protein, a bacterial surface protein and/or a tetrathionate reductase, or a fragment thereof, may be harmonized for expression in a host microorganism, such as a probiotic bacterium. Techniques for harmonization of a sequence to account for differences in codon usage across species in order to improve the level of protein expression are described herein or known in the art.

Recombinant probiotic bacteria, as described herein, may be provided alone or in combination with other compounds or probiotic bacteria, in any pharmaceutically acceptable carrier, in a form suitable for administration to a subject, to increase colonization of the probiotic bacterium in the gastrointestinal tract of a subject, reduce inflammation in the gastrointestinal tract of a subject and/or treat or prevent inflammatory bowel disease in a subject in need thereof. In some embodiments, a recombinant probiotic bacterium expressing a GbpA protein, as described herein, may be administered in combination with a recombinant probiotic bacterium expressing a tetrathionate reductase, as described herein. In some embodiments, a recombinant probiotic bacterium expressing a GbpA protein, as described herein, and a tetrathionate reductase, as described herein, may be administered to a subject in need thereof. If desired, treatment with a recombinant probiotic bacterium according to the present disclosure may be combined with more traditional and existing therapies for gastrointestinal inflammation or inflammatory bowel disease. A recombinant probiotic bacterium according to the present disclosure may be provided chronically or intermittently. “Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Conventional pharmaceutical practice may be employed to provide suitable formulations or compositions to administer a recombinant probiotic bacterium according to the present disclosure to a subject suffering from or presymptomatic for gastrointestinal inflammation or inflammatory bowel disease. Any appropriate route of administration may be employed, for example, oral administration, or rectal administration. For oral administration, formulations may be in the form of tablets or capsules. For rectal administration, formulations may be in the form of suppositories or enemas. Methods well known in the art for making formulations are found in, for example, “Remington's Pharmaceutical Sciences” (19^{th} edition), ed. A. Gennaro, 1995, Mack Publishing Company, Easton, Pa.

For therapeutic or prophylactic compositions, a recombinant probiotic bacterium according to the present disclosure may be administered to an individual in an amount sufficient to stop or slow gastrointestinal inflammation or inflammatory bowel disease. An “effective amount” of a compound according to the invention includes a therapeutically effective amount or a prophylactically effective amount. A “therapeutically effective amount” refers to an amount effective, at dosages and for periods of time necessary, to achieve the desired therapeutic result, such as amelioration of gastrointestinal inflammation or inflammatory bowel disease. A therapeutically effective amount of a recombinant probiotic bacterium according to the present disclosure may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any detrimental or side effects of the recombinant probiotic bacterium according to the present disclosure are outweighed by the therapeutically beneficial effects. A “prophylactically effective amount” refers to an amount effective, at dosages and for periods of time necessary, to achieve the desired prophylactic result, such as amelioration of gastrointestinal inflammation or inflammatory bowel disease. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of a recombinant probiotic bacterium according to the present disclosure in a composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

As used herein, a subject may be a human, non-human primate, rat, mouse, cow, horse, pig, sheep, goat, dog, cat, etc. The subject may be a clinical patient, a clinical trial volunteer, an experimental animal, etc. The subject may be suspected of having or at risk for having gastrointestinal inflammation or inflammatory bowel disease, be diagnosed with gastrointestinal inflammation or inflammatory bowel disease, or be a control subject that is confirmed to not have gastrointestinal inflammation or inflammatory bowel disease. Diagnostic methods for gastrointestinal inflammation or inflammatory bowel disease and the clinical delineation of such diagnoses are known to those of ordinary skill in the art.

In some embodiments, the subject may be benefited by increased colonization and/or persistence of a recombinant probiotic bacterium in the gastrointestinal tract. Determination and monitoring of colonization and/or persistence of a recombinant probiotic bacterium in the gastrointestinal tract may be done using standard techniques, such as by obtaining a sample (such as a stool sample) from a subject and determining the presence, absence or amount of a recombinant probiotic bacterium by amplification of a nucleic acid sequence unique to the recombinant probiotic bacterium.

The present invention will be further illustrated in the following examples.

Materials and Methods

Bacterial Strains and Growth Conditions

E. coli strains and S. typhimurium SL1344 were routinely cultivated in liquid Luria-Bertani-Miller (LB) media or plates with 1.8% w/w agar. For some experiments, strains were cultivated in minimal M9 medium (64 g/L Na₂HPO₄·7H₂O, 15 g/L KH₂PO₄, 2.5 g/L NaCl, 5 g/L NH₄C, 2 mM MgSO4, 0.1 mM CaCl₂)). Media were supplied with ampicillin (Ap; 100 ug/ml), tetracycline (Tc; 12.5 ug/ml for all of the strains, except 4 ug/ml for E. coli Nissle attB^{phi80}::ttrACBSR), Kanamycin (Km; 40 ug/ml). Strains, plasmids and primers used in the construction of the E. coli Nissle attB^{phi80}::ttrACBSR strain are described in Table 1.

TABLE 1
Strains, plasmids and primers used in the construction of E. coli Nissle
attB^{phi80}::ttrACBSR

| | | |
|---|---|---|
| | | Source/ |
| Name | Description | SEQ ID NO |

Strains

| | | |
|---|---|---|
| Salmonella enterica | Wild-type | Gibson |
| subsp. enterica | | laboratory |
| serovar Typhimurium | | |
| SL1344 | | |
| Escherichia coli | Wild-type | MUTAFLOR ® |
| Nissle 1917 | | |
| E. coli Nissle | Derivative of Escherichia coli Nissle 1917 with | SEQ ID NO: 47 |
| attB^{phi80}::ttrACBSR | pAH162-ttrACBSR plasmid integrated to attB-site | |
| | of phage phi 80 | |
| E. coli Nissle | Derivative of E. coli Nissle attB^{phi80}::ttrACBSR | SEQ ID NO: 48 |
| attB^{phi80}::Km^{R} | with deletion of ttrACBSR operon | |
| BW23473 | Pir⁺ strain, required for propagation of CRIM | CGSC |
| | pAH162 plasmid and its derivatives (Haldimann | |
| | et al. (2001) J. Bacteriol. 183:6384-93) | |

Plasmids

| | | |
|---|---|---|
| pAH162 | conditional replication, integration and modular | isolated from |
| | (CRIM) plasmid, carries phage phi 80 attP-site | CGSC 7873 |
| | and Tc-resistance cassette (Haldimann et al. | strain |
| | (2001) J. Bacteriol. 183:6384-93) | |
| pAH123 | thermo sensitive helper plasmid, carrying phage | isolated from |
| | phi 80 int gene behind phage Lambda Pr | CGSC 7861 |
| | promoter under cl857 control; required for | strain |
| | integration of pAH162 (Haldimann et al. (2001) | |
| | J. Bacteriol. 183:6384-93) | |
| pAH162-ttrACBSR | pAH162 with ttrACBSR operon cloned | SEQ ID NO: 46 |
| pKD46 | thermo sensitive, carries the A red genes behind | isolated from |
| | the araBAD promoter | CGSC 7669 |
| | | strain |

Primers

| | | SEQ ID NO: |
|---|---|---|
| ga1 (Primer to | gagctcgaattctcatgtttg | 1 |
| amplify the | | |
| pAH162 plasmid | | |
| backbone) | | |
| ga2 (Primer to | ggatcctctagagtcgacctg | 2 |
| amplify the | | |
| pAH162 plasmid | | |
| backbone) | | |
| ga3 (Primer to | gcatgcctgcaggtcgactctagaggatccgttatatacgctcga | 3 |
| amplify ttrACBSR | tttttgc | |
| from S. | | |
| Typhimurium | | |
| SL1344 (bold font | | |
| denotes region of | | |
| primer binding to | | |
| ttr) | | |
| ga4 (Primer to | ataagctgtcaaacatgagaattcgagctcttattcatggctcata | 4 |
| amplify ttrACBSR | cgttg | |
| from S. | | |
| Typhimurium | | |
| SL1344 (bold font | | |
| denotes region of | | |
| primer binding to | | |
| ttr) | | |
| ga5 (Primer to | cgttatggactgcaacatgg | 5 |
| confirm ttr | | |
| integration into | | |
| pAH162 plasmid) | | |
| ga6 (Primer to | gcaaacggcctaaatacagc | 6 |
| confirm ttr | | |
| integration into | | |
| pAH162 plasmid) | | |
| ga7 (Primer to | tgccaagcttgcatgcctgcaggtcgactctagaggatccattcc | 7 |
| amplify | ggggatccgtcgacc | |
| Kanamycin- | | |
| resistance | | |
| cassette) | | |
| ga8 (Primer to | ctgatcagtgataagctgtcaaacatgagaattcgagctctgtag | 8 |
| amplify | gctggagctgcttcg | |
| Kanamycin- | | |
| resistance | | |
| cassette) | | |

L. reuteri DSM20016 strain and its derivatives were routinely cultivated in liquid MRS media without agitation or plates with the same media supplemented with 1.8% w/w agar in anoxic conditions of anaerobic jar. E. coli DH5α strain was cultivated in LB, SOB or SOC media. Media was supplied with Erythromycin (Erm; 5 ug/ml for L. reuteri, 150 ug/ml for E. coli).

Molecular Biology Techniques

PCR fragments for cloning were generated using Q5 High Fidelity DNA polymerase (NEB) unless otherwise noted and oligonucleotide primers were from IDT Inc., Vancouver, BC. Qiagen (Hilden, Germany) products were used for the isolation of plasmid or chromosome DNA and purification of PCR fragments.

Strain Construction of E. coli Nissle attB^{phi80}::ttrACBSR

The ttrACBSR operon (SEQ ID NO: 25) of S. typhimurium SL1344 was cloned to CRIM plasmid pAH162 (SEQ ID NO: 45) by Polymerase Incomplete Primer Extension technique (Klock H E et al. 2008 Proteins 71:982-994) and the plasmid was subsequently integrated into phi80-phage attachment site on the chromosome of E. coli Nissle as described (Haldimann A and Wanner B L 2001 J Bacteriol 183:6384-6393). Briefly, ttrACBSR was amplified with ga3/ga4 primers (SEQ ID NO: 3 and 4) and pAH162 μlasmid backbone was amplified with ga1/ga2 (SEQ ID NO: 1 and 2) primers using Q5 High-Fidelity polymerase (New-England Biolabs) according to the manufacturer's instructions with chromosomal DNA as a template. The obtained PCR products were combined and transformed into E. coli BW23473. Several resulting plasmids were tested for functionality in growth competition assays and one plasmid was selected. E. coli Nissle/pAH123, cultivated at 30° C., was transformed with the selected plasmid and outgrowth continued at 37° C. The resulting chromosomal integration of the plasmid was confirmed by PCR.

For construction of the control E. coli Nissle attB^{Ph}i80::KmR strain, the phage-Lambda Red recombinase-mediated recombination-based method was employed as described (Datsenko K A and Wanner B L 2000 Proc Nat Acad Sci USA 97:6640-6645). A Kanamycin-resistance cassette was amplified with ga7/ga8 (SEQ ID NO: 7 and 8) primers using the chromosome of E. coli JW4283-3 as a template. The resulting PCR-fragment was introduced into E. coli Nissle attB^{phi80}::ttrACBSR/pKD46 and the resulting strain cultivated in the presence of L-arabinose (Datsenko K A and Wanner B L 2000 Proc Nat Acad Sci USA 97:6640-6645). The structure of the resulting E. coli Nissle attB^{phi80}::Km^{R} strain was confirmed by PCR with ga7/ga8 (SEQ ID NO: 7 and 8) primers by the presence of amplification of the corresponding fragment.

Growth Characteristics of the E. coli Nissle attB^{phi80}::ttrACBSR Strain

Growth Competition Assay

Cultures of tested strains (E. coli Nissle and E. coli Nissle attB^{phi80}::ttrACBSR, or E. coli BW23473 and E. coli BW23473/pAH162-ttr) were inoculated with overnight cultures of the corresponding strain (1/50) and incubated until they reached OD₆₀₀=0.55-0.7. The subcultures were dissolved to similar optical densities, mixed and then dissolved to OD₆₀₀=0.05 with media, which did or did not contain potassium tetrathionate (30 mM). Mixed cultures were incubated without agitation in media-filled capped tubes overnight. The next day, cultures were dissolved and plated onto selective (Tc) and non-selective plates to count modified/unmodified colonies.

Tetrathionate Reduction Assay

M9 media (+0.2% w/w glycerol, 30 mM potassium tetrathionate) was inoculated with fresh cultures of modified or wild-type strains in the same media (1/100) and incubated overnight with agitation or in media-filled closed test tubes with no agitation. The next day, cultures were centrifuged (12000 g, 2 min) and thiosulfate concentration in the supernatant was estimated by neutral iodimetric titration. Concentration of consumed tetrathionate was estimated based on the fact that one molecule of tetrationate is converted into two thiosulfate molecules by ttr operon enzyme activity (Hensel et al. 1999 Mol Microbiol 32:275-287).

L. reuteri Strain Construction

The L. reuteri lar_0958::gbpA₂₄₋₂₀₃ strain, also referred to herein as. L. reuteri::GbpA, was constructed as follows.

Construction of pG+host-MBP-gbpA plasmid was performed using Gibson Assembly Master Mix (NEB) and Q5 High Fidelity DNA polymerase (NEB). pG+host5 μlasmid (SEQ ID NO: 43) was kindly provided by Dr. John K. McCormick (Lia et al. (2011) PNAS 108:3360-3365). GbpA N-terminal domain coding sequence was synthesized by IDT DNA with sequence optimization for expression in L. reuteri (SEQ ID NO: 22) by harmonization algorithm (as described by Angov et al. (2011) Mol Microbiol 705(1):1-13). Table 2 shows the primers used in the construction of the recombinant probiotic strain expressing a fragment of the GbpA protein. The fragment encoding N-terminal part of L. reuteri mucus-binding protein (MBP) was amplified with primers 1 and 2 (SEQ ID NO: 9 and 10), fragment encoding N-terminal domain of Vibrio cholerae GbpA protein was amplified with primers 3 and 4 (SEQ ID NO: 11 and 12), fragment encoding C-terminal part of L. reuteri MBP was amplified with primers 5 and 6 (SEQ ID NO: 13 and 14), plasmid backbone of pG+host5 was amplified with primers 7 and 8 (SEQ ID NO: 15 and 16). All the amplified fragments were mixed and Gibson Assembly reaction was performed according to the manufacturer's instruction. After reaction was performed, the mix was transformed to E. coli DH5α strain by electroporation. The structure of resulting plasmid was confirmed by PCR with several sets of primers, flanking each region, and sequencing.

pG+host-MBP-gbpA was electroporated to L. reuteri DSM20016 strain. Strain was cultivated at 30 C to enable plasmid replication, then diluted and cultivated at 37 C overnight to obtain population of single-crossover integrants. Integration was confirmed by PCR and sequencing. Integrants had Erm^{R} phenotype with no mutations found in pG+host-MBP-gbpA on found by sequencing. The single crossover integrants were cultivated at 30 C overnight without antibiotic to obtain double-crossover integrants, then plated on non-selective plates to single colonies. Several colonies were transferred by toothpicks to Erm-agar plate and Erm—sensitive clones were isolated. Double-crossover integrants were found by PCR, the sequence was confirmed by sequencing.

Double crossover homologous integration technique was employed for strain construction. First pG+host-LAR-gbpA plasmid (SEQ ID NO: 44) was extracted from E. coli strain and transformed to L. reuteri. An electroporation protocol with modifications was used. Briefly, 1/20 inoculum of overnight culture of L. reuteri was inoculated in MRS broth+1% glycine as described in Wei et al. (Wei et al. (1995) J. Microbiol Methods. 21:97-109). Once OD₆₀₀ reached 0.2-0.3, bacteria were left on ice for 10 minutes to stop growth. Bacteria were then washed twice with ice-cold water, once in ice-cold 0.3M sucrose, and then re-suspended in 1/50 volume of 0.3M sucrose. Electroporation was performed on ice using a 1 mm electroporation cuvette with a BTX ECM 399 electroporation system. 4 ul of extracted pG+host-LAR-gbpA plasmid with 16 ul electrocompetent L. reuteri cells and 20 ul of electroporation buffer (0.3M sucrose) was electroporated at 1290V. Cell and plasmid mixture was immediately transferred to 2 mL pre-warmed 37° C. MRS broth and incubated for 2 hours under anaerobic conditions. 70 ul of cells were plated on 1.8% MRS agar plates supplemented with 5 ug/ml Erm. After 62 hours of anaerobic incubation, 3 colonies resulted. Colonies were selected and plated on 1.8% MRS agar plates supplemented with 5 ug/ml Erm. Integration of plasmid was confirmed using primers 9 and 10 (SEQ ID NO: 17 and 18) for L. reuteri backbone and primers 3 and 4 (SEQ ID NO: 11 and 12) for N-terminal domain of Vibrio cholerae gbpA protein.

TABLE 2
Primers used in the construction of the recombinant L. reuteri::GbpA

| Primer name | Sequence | SEQ ID NO: |
|---|---|---|
| Primer 1 (Primer to amplify | ccaattactaccagcttcagcactacc | 9 |
| DNA fragment encoding the | agcactaccaatcctctttcggtaata | |
| N-terminal region of L. reuteri | aatctt | |
| mucus-binding protein | | |
| (MBP) (italicized sequence | | |
| encodes flexible peptide | | |
| linkers added between MBP | | |
| and GbpA) | | |
| Primer 2 (Primer to amplify | gtgagcgcgcgtaatacgactcacta | 10 |
| the DNA fragment encoding | tagggcggatccggtctatcctttatgg | |
| the N-terminal region of L. | gagaac | |
| reuteri mucus-binding | | |
| protein (MBP)) | | |
| Primer 3 (Primer to amplify | gtgctgaagctggtagtaattggagtc | 11 |
| the DNA fragment encoding | atccacaatttgaaaaaggtagtgct | |
| the N-terminal domain of | ggtagtgct | |
| Vibrio cholera GbpA protein | gctggtagtcacggttacgtatcggca | |
| (italicized sequence encodes | g | |
| flexible peptide linkers added | | |
| between MBP and GbpA; | | |
| underlined sequence | | |
| denotes strep-tag II)) | | |
| Primer 4 (Primer to amplify | aattcaccactaccagcagcactacc | 12 |
| the DNA fragment encoding | agcactaccaccgtcaaacttaacgt | |
| the N-terminal domain of | caataacg | |
| Vibrio cholera GbpA protein | | |
| (italicized sequence encodes | | |
| flexible peptide linkers added | | |
| between MBP and GbpA)) | | |
| Primer 5 (Primer to amplify | agtgctggtagtgctgctggtagtggt | 13 |
| the DNA fragment encoding | gaatttaaagttacctatagtggtagtg | |
| the C-terminal region of L. | acagc | |
| reuteri MBP (italicized | | |
| sequence encodes flexible | | |
| peptide linkers added | | |
| between MBP and GbpA)) | | |
| Primer 6 (Primer to amplify | cgatatcaagcttatcgataccgtcga | 14 |
| the DNA fragment encoding | cctcgagaattcccgtcaagataatc | |
| the C-terminal region of L. | cgataag | |
| reuteri MBP) | | |
| Primer 7 (Primer to amplify | gaattgggtaccgggccccccctcg | 15 |
| the plasmid backbone of | agg | |
| pG + host5) | | |
| Primer 8 (Primer to amplify | gccctatagtgagtcgtattacgcgcg | 16 |
| the plasmid backbone of | c | |
| pG + host5) | | |
| Primer 9 (Primer to confirm | aactgttggggttacttcggta | 17 |
| integration of pG + host-LAR- | | |
| gbpA plasmid into L. reuteri | | |
| backbone) | | |
| Primer 10 (Primer to confirm | ctggttgttgctcaggtgttt | 18 |
| integration of pG + host-LAR- | | |
| gbpA plasmid into L. reuteri | | |
| backbone) | | |

### Colitis Animal Trials

C57BL/6 female mice (Jackson Laboratories, Bar Harbor, Maine) were maintained in pathogen free conditions at the Bioscience Facility at the University of British Columbia Okanagan (UBCO), Kelowna, BC. They were bred in house and caged in a temperature controlled (22±2° C.) room with 12-hour light/dark cycle. They were fed irradiated food and sterile tap water. Post-weaned female offspring were weaned at 4 weeks and then assigned of three groups: no probiotic, modified designer probiotic, or unmodified parent probiotic. Probiotic groups received 100 pL (3×10¹² CFU/mL when testing the parental and recombinant strain expressing the ttr operon and 2×10⁹ cfu/mL when testing the parental and recombinant strain expressing the GbpA fragment) of the probiotic via oral gavage administered once per day for a period of three days for the E. coli strains and one gavage for the L. reuteri strains. The third treatment group served as the control group and received no oral gavage or probiotic supplementation.

Mice were then exposed to 3.5% DSS via drinking water and monitored throughout the 7-day exposure for mortality/morbidity Mice were immediately euthanized if they showed signs of distress due to gavage such as: lethargy, hunched posture, difficulty breathing, blood emerging from the mouth and/or nose or a loss in total body weight ≥20%. Mice were sacrificed at day 7. Body weight was measured every day during the 7 day DSS exposure. Body weight data is presented as percentage of the initial body weight. Probiotic supplemented groups were exposed to DSS and no DSS. A DSS control with no supplementation was also used to provide a control for the DSS-induced colitis.

In a second set of trials, C57BL/6 (Jackson Laboratories, Bar Harbor, Maine) and Muc2^{-/-} male and female mice (Morampudi V, et al. Mucosal Immunology. 2016:1-16) were maintained in pathogen free conditions at the Bioscience Facility at the University of British Columbia Okanagan (UBCO), Kelowna, BC. They were bred in house and caged in a temperature controlled (22±2° C.) room with 12-hour light/dark cycles, fed irradiated food, and sterile tap water. The protocols used were approved by the University of British Columbia's Animal Care Committee and in direct accordance with guidelines drafted by the Canadian Council on the Use of Laboratory Animals. C57BL/6 offspring were weaned at 19-21 days of age and Muc2^{-/-} offspring were weaned at 28-30 days of age. Mice were then assigned one of three groups: no probiotic, modified designer probiotic, or unmodified parent probiotic. Probiotic groups received 100 pL (3×10¹² CFU/mL when testing the parental and recombinant strain expressing the ttr operon and 2×10⁹ cfu/mL when testing the parental and recombinant strain expressing the GbpA fragment) of the probiotic. For Muc2^{-/-} spontaneous colitis, mice were gavaged once weekly for 4 consecutive weeks. Since Muc2^{-/-} spontaneous colitis progresses with age, 2 time points were used when testing the E. coli strains and for the Muc2^{-/-} control. One cohort of the mice was taken out to 3 months of age and then sacrificed and a second cohort was taken out to 4 months of age and then sacrificed. Mice were monitored daily and weighed weekly to score and check for colitis disease progression. Mice were immediately euthanized if they developed rectal prolapse or total clinical score of 11 or greater.

In the second set of trials, for DSS-induced colitis, mice were administered probiotics via oral gavage once per day for a period of three days for testing parental and recombinant strain expressing ttr operon. Mice were administered probiotics only once for testing the parental and recombinant strain expressing GbpA. The third treatment group served as the control group and received no oral gavage or probiotic supplementation. Mice were then exposed to 3.5% DSS via drinking water and monitored throughout the 7-day exposure for mortality/morbidity. Mice were immediately euthanized if they showed signs of distress due to gavage such as: lethargy, hunched posture, difficulty breathing, blood emerging from the mouth and/or nose or a loss in total body weight ≥20%. Mice were sacrificed at day 7. Body weight was measured every day during the 7 day DSS exposure. Probiotic supplemented groups were exposed to DSS and no DSS. A DSS control with no supplementation was also used to provide a control for the DSS-induced colitis. A further 5-ASA group of mice were treated with 5-aminosalicylates (5-ASA) (Sigma-Aldrich), receiving 75 mg/kg/day, a remission induction dose similar to the daily dose in humans.

Body Weight and Clinical Scores

In the second set of trials, for DSS-induced colitis, body weight data is presented as percentage of weight change of the initial body weight. For Muc2^{-/-} spontaneous colitis, body weight data is presented as a percentage of weight change from each consecutive week.

Mice were scored based on their body movement, rectal bleeding, stool consistency, weight change, and hydration. For DSS-induced colitis; for body movement, a score of 2 was given for piloerection and a 2 for reduced movement, a score of 3 for hunched posture and a 3 for inactive, and a score of 5 was given for shaking. For rectal bleeding, a score of 1 was given for a positive fecal occult blood test, 2 for blood in the stool, 3 for large amount, and 4 for extensive blood in stool and visible blood at anus. For stool consistency, a score of 1 was given for loose stool, 2 for watery stool, 3 for diarrhea, and a 4 for no formed stool. For weight, a score of 1 was given for loss of 5-10% of initial weight, a 2 for 10-15%, and weight loss of more than 15% was given a 3. For hydration, a score of 1 was given for slight sunken eyes, 3 for dehydrated eyes, and a 4 for a skin tent. All scores from each category were tallied and a final clinical score per day for each mouse was given during the DSS treatment. Higher clinical scores correlated with increased intestinal inflammation.

For Muc2^{-/-}, for body movement, a score of 2 was given for piloerection and a 2 for reduced movement, a score of 3 for hunched posture and a 3 for inactive, and a score of 5 was given for shaking. For rectal bleeding, a score of 1 was given for rectal swelling, a score of 2 for visible blood in the stool, a score of 3 for large amount of blood in stool and/or cage, a score of 4 for blood in stool and anus, and a score of 4 for rectal prolapse. For stool consistency, a score of 1 was given for soft stool, and a score of 2 for diarrhea. For weight loss, a score of 1 was given for loss of up to 5%, a score of 2 for 5-10%, a score of 3 for loss of 10-19%, and a score of 5 for loss of more than 20%. For hydration, a score of 1 was given for slight sunken eyes, 3 for dehydrated eyes, and a 4 for a skin tent. All scores from each category were tallied and a final clinical score per week for each mouse was given during the Muc2^{-/-} spontaneous colitis. A total clinical score of 11 or greater or rectal prolapse indicated immediate euthanization.

Tissue Collection

Mice were first anesthetized with isofluorane and then euthanized by asphyxiation by CO₂ and then cervical dislocation; the distal colon, ileum, and cecum were removed and immersed in 1 mL of RNA-later (Qiagen) and stored at −80° C. for RNA extractions and quantitative polymerase chain reaction (qPCR) cytokine analysis or immersed in 1 mL of 10% neutral buffered formalin (Thermo Fisher Scientific) at 4° C. for histological analyses and immunofluorescence.

In the second set of trials, for DSS-induced colitis, mice were first anesthetized with isofluorane, sacrificed by asphyxiation by CO₂, and then followed by cervical dislocation. For Muc2^{-/-} spontaneous colitis, mice were first anesthetized with isofluorane, and then blood was withdrawn using intracardiac puncture and then cervical dislocation. Cardiac puncture was used a terminal end-point.

The distal colon, ileum, and cecum were removed and sectioned into 3 pieces. One section was immersed in 1 mL of RNAlater (Qiagen) and stored at −80° C. for RNA extractions and quantitative polymerase chain reaction (qPCR) cytokine analysis, second section was immersed in 1 mL of 10% neutral buffered formalin (Thermo Fisher Scientific) at 4° C. for histological analyses and immunofluorescence, and the third section was flash frozen in LN2 (liquid nitrogen) for microbial analysis. For Muc2^{-/-} colitis, the mesenteric lymph nodes (MLN) and spleen were collected and stored in 1 mL of sterile 1×PBS (Lonza).

Histopathological Scoring

In the second set of trials, for histology, tissue sections were placed in 10% neutral-buffered formalin, left overnight at 4° C., and then transferred into 70% ethanol after 2 1×PBS washes. These sections were paraffin embedded and cut into 5 μm sections onto slides. A slide was stained for Hematoxylin and eosin stain (H&E) staining for histopathological scoring. Paraffin-embedded colon cross sections were stained using H&E staining and damage scores measured. The histopathology scores were based on 4 parameters. Scores were determined as: crypt damage (0=intact, 1=loss of 1/3 basal, 2=loss of 2/3 basal, 3=entire crypt loss, 4=change of epithelial surface with erosion, 5=confluent erosion); ulceration (0=absence of ulcers, 1=1 or 2 foci of ulcerations, 2=3 or 4 foci of ulcerations, 3=confluent or extensive ulcerations); inflammation (0=normal, 0.5=very minimal, 1=minimal, 2=mild, 3=moderate, 4=marked, 5=severe); and goblet cell depletion (0=>50, 1=25-50, 2=10-25, 3=<10). Scores in each category were added up and a total histopathological score was given. Slides were viewed on an Olympus IX81 fluorescent microscope at 200× magnification. Histopathological images were taken on MetaMorph software.

Immunoflourescence

For the second set of trials, paraffin-embedded tissue sections were deparaffinized and antigen retrieval of rehydrated tissues was performed using 1 mg/ml trypsin (Sigma Aldrich) followed by incubation with primary antibodies. Slides were incubated with either rat monoclonal IgG₂a antibody raised against F4/80 (Cedarlane) to examine macrophages or rabbit polyclonal antibody IgG raised against MPO-1 (Thermo Fisher Scientific) to examine polymorphonuclear leukocytes. This was followed by secondary antibodies of goat anti-rabbit IgG ALEXA FLUOR-conjugated 594-red antibody (Invitrogen) or goat anti-rabbit IgG ALEXA FLUOR-conjugated 488-green antibody (Invitrogen). Tissue sections were mounted using DAPI (Sigma Aldrich) and viewed on an Olympus IX81 fluorescent microscope at 200× magnification. For inflammatory cell counts, positive cells were quantified in the sub-mucosal region by a blinded observer and verified by another from a stitched image using METAMORPH software. The total number of positive cells in all sub-mucosal regions per mouse tissue were tallied.

mRNA Extraction, cDNA Synthesis, and Cytokine Analysis

Total RNA was purified using Qiagen RNEASY kits (Qiagen) according to the manufacturer's instructions. Extracted RNA was purified using Oligo (dT) purification of mRNA using DYNABEADS mRNA purification kit (Invitrogen). 5-10 μg of DSS-exposed total RNA (estimated to contain 5000 ng of mRNA) was used with 0.25 mg of DYNABEADS Oligo (dT)25 in a total volume of 200 μl (including buffers). The beads were washed with buffers according to the manufacturer's instructions. This was eluted in 20 μl of Tris-HCl and 7.5 μl of this elute was used for cDNA synthesis. DNA was synthesized with ISCRIPT cDNA Synthesis Kit (Bio-rad Laboratories). Quantitative PCR (qPCR) was performed in duplicates in a volume of 10 μl with SSO FAST EVA GREEN Supermix (Bio-rad Laboratories) on the Biorad CFX 96 real time PCR detection system with cycling conditions previously described (Baker J et al. 2012 Am J Physiol Gastrintest Liver Physiol 303(7):G825-G836). All primers were synthesized by the Integrated DNA Technology (IDT), Canada. Primer efficiencies were verified according to the Minimum Information for Publication of Quantitative Real-Time PCR Experiments guidelines. The specificity of the primers was verified by using Bio-rad CFX software and efficiencies were determined using standard curves. Expression of 18S and GAPDH were used as reference genes for gene expression analysis carried out using CFX manager software version 1.6.541.1028 (Bio-rad Laboratories).

For the second set of trials, total RNA was purified using Qiagen RNEASY kits (Qiagen) according to the manufacturer's instructions. Extracted RNA was purified using Oligo (dT) purification of mRNA using DYNABEADS mRNA purification kit (Invitrogen). 5-10 μg of DSS-exposed total RNA (estimated to contain 5000 ng of mRNA) was used with 0.25 mg of DYNABEADS Oligo (dT)25 in a total volume of 200 μl (including buffers). The beads were washed with buffers according to the manufacturer's instructions. This was eluted in 20 μl of Tris-HCl and 7.5 μl of this elute was used for cDNA synthesis. DNA was synthesized with ISCRIPT cDNA Synthesis Kit (Bio-rad Laboratories). Quantitative PCR (qPCR) was performed in duplicates in a volume of 10 μl with SSO FAST EVA GREEN Supermix (Bio-rad Laboratories) on the Biorad CFX 96 real time PCR detection system with cycling conditions previously described (Baker J et al. 2012 Am J Physiol Gastrintest Liver Physiol 303(7):G825-G836). All primers were synthesized by the Integrated DNA Technology (IDT), Canada. Primer efficiencies were verified according to the Minimum Information for Publication of Quantitative Real-Time PCR Experiments guidelines. The specificity of the primers was verified by using Bio-rad CFX software and efficiencies were determined using standard curves. Expression of 18S, TATA-binding protein (TBP), and eukaryotic elongation factor 2 (EEF2) were used as reference genes for gene expression analysis carried out using CFX manager software version 1.6.541.1028 (Bio-rad Laboratories). Table 3 includes a list of primer sequences used for qPCR.

TABLE 3
Primers used for mRNA cytokine analysis for qPCR

| Primer | Forward Primer | Reverse Primer |
|---|---|---|
| 18S | CGGCTACCACCCAAGGAA | GCTGGAATTACCGCGGCT |
| | (SEQ ID NO: 54) | (SEQ ID NO: 55) |
| TBP | ACCGTGAATCTTGGCTGTAAC | GCAGCAAATCGCTTGGGATTA |
| | (SEQ ID NO: 56) | (SEQ ID NO: 57) |
| EEF2 | TGTCAGTCATCGCCCATGTG | CATCCTTGCGAGTGTCAGTGA |
| | (SEQ ID NO: 58) | (SEQ ID NO: 59) |
| TNF-α | CATCTTCTCAAAATTCGAGTGACA | TGGGAGTAGACAAGGTACAACCC |
| | (SEQ ID NO: 60) | (SEQ ID NO: 61) |
| IFN-γ | TCAAGTGGCATAGATGTGGAAGA | TGGCTCTGCAGGATTTTCATG |
| | (SEQ ID NO: 62) | (SEQ ID NO: 63) |
| IL-10 | AGGGCCCTTTGCTATGGTGT | TGGCCACAGTTTTCAGGGAT |
| | (SEQ ID NO: 64) | (SEQ ID NO: 65) |
| IL-1β | AGCTTCCTTGTGCAAGTGTC | CCCTTCATCTTTTGGGGTCC |
| | (SEQ ID NO: 66) | (SEQ ID NO: 67) |
| IL-17a | TCCCTCTGTGATCTGGGAAG | CTCGACCCTGAAAGTGAAGG |
| | (SEQ ID NO: 68) | (SEQ ID NO: 69) |
| Reg3γ | CCCGTATAACCATCACCATCAT | GGCATCTTTCTTGGCAACTTC |
| | (SEQ ID NO: 70) | (SEQ ID NO: 71) |
| Muc2 | GCCAGATCCCGAAACCA | TATAGGAGTCTCGGCAGTCA |
| | (SEQ ID NO: 72) | (SEQ ID NO: 73) |

SCFA Analysis

The amount of short chain fatty acids (SCFA) were analyzed in cecal samples by gas chromatography (with modifications) Zhao G, et al. Biomedical Chromatography. 2006; 20(8):674-682. Cecal tissue samples were homogenized with 700 μl isopropyl alcohol, containing 2-ethylbutiric acid at 0.01% v/v as internal standard at 30 Hz for 13 minutes in a homogenizer (Retsch Metal Beads MixerMill MM 400) with stainless steel metal beads. Samples were kept at room temperature for 15 minutes and then centrifuged in a MEGAFUGE 40R (Thermo Fisher) at 15,100×g for 10 minutes at 4° C. Resulting supernatant was collected and the procedure was repeated for a second time on the leftover pellet to confirm complete extraction. 0.9 μl of the cleared supernatant was directly injected to a Trace 1300 Gas Chromatograph in splitless mode, that is equipped with a Flame-ionization detector, and an A11310 auto sampler (Thermo Fisher Scientific). A fused silica FAMEWAX (Restek Cat #12498) column 30 m×0.32 mm i.d. coated with 0.25 μm film thickness was used. Helium was supplied as the carrier gas at a flow rate of 1.8 mL/min. The initial oven temperature was 80° C., maintained for 5 minutes, rose to 90° C. at 5° C./min, then increased to 105° C. at 0.9° C./min, and finally increased to 240° C. at 20° C./min and held for 5 minutes. The temperature of the FID and the injection port was 240 and 230° C., respectively. The flow rates of hydrogen, air and nitrogen as makeup gas were 30, 300 and 20 mL/min, respectively. Data analysis was carried out with CHROMELEON 7 software. Peaks were analyzed on software and the area under peaks for acetic, propionic, and butyric acid data was represented as weight percentage of the total cecal tissue.

### Example 1—Construction of a Recombinant Probiotic Strain Expressing the ttr Operon and Analysis of Growth In Vitro

A recombinant probiotic strain of E. coli Nissle was genetically engineered to express the tetrathionate respiratory operon as described herein. The PCR amplification of the long ttr operon (7.4 kb) (Gene ID: 1252901 in NCBI Gene), even with a high-fidelity polymerase, might result in random mutations possibly interfering with the proper function of the enzymes. To select for the best pAH162-ttr plasmid for subsequent chromosomal integration, a growth competition assay and a thiosulfate production assay were performed. During the growth competition assay, a strain bearing ttr operon (on plasmid or integrated into the chromosome) and its parental strain were incubated simultaneously in the same liquid culture without aeration. After the inoculation of the culture with the mixture of tested strains, tetrathionate solution or water (as a control) were added to determine whether the ttr-bearing strain had a growth advantage in the presence of tetrathionate and if this advantage is enough to outcompete the parental strain. Resistance of the ttr-bearing strain to Tc was employed to estimate its numbers. FIG. 8 shows that the percentage of Tc-resistant colonies (E. coli Nissle attB^{phi80}::ttrACBSR) in mixed culture is higher in the presence of tetrathionate and that the ttr operon integration resulted in a growth advantage during simultaneous cultivation. The competition coefficients (percent of Tc-resistant colonies in the presence of tetrathionate divided by the percent of Tc-resistant colonies in the absence of tetrathionate) for the mixture of E. coli BW23473, bearing pAH162-ttr plasmid, with wild type strain and for the mixture of E. coli Nissle with chromosome-integrated ttr operon mixed with the wild type strain were similar. This shows that a single copy of the ttr operon is sufficient to induce the same effect as several copies in the case of plasmid-based expression.

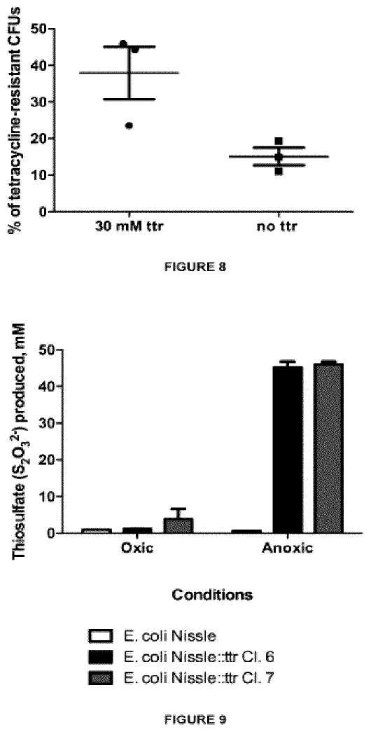

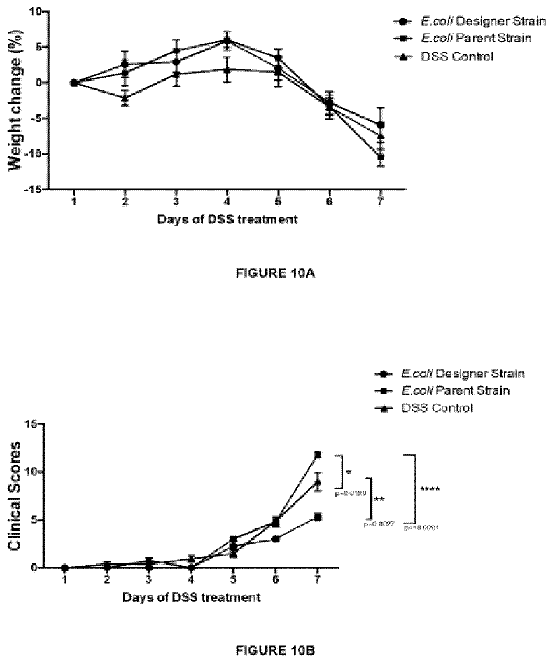

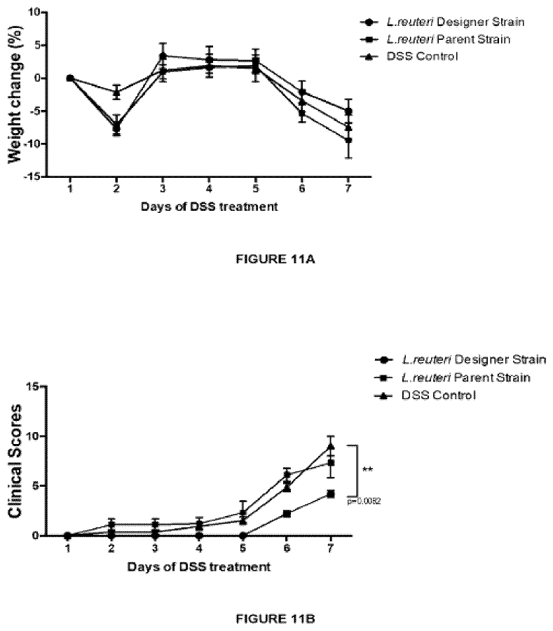

To determine if E. coli Nissle strain with the integrated ttr operon is capable of reducing tetrathionate, a thiosulfate production assay was performed. This assay is based on the colorimetric estimation of the concentration of thiosulfate—a product of tetrathionate reduction. Wild type E. coli Nissle and modified E. coli Nissle attB^{phi80}::ttrACBSR were grown in media containing 30 mM potassium tetrathionate under oxic or anoxic conditions. The consumption of tetrathionate in each condition was estimated based on the amount of thiosulfate produced using a conversion factor of one molecule of tetrationate converts to two thiosulfate molecules (described herein). FIG. 9 shows that modified E. coli Nissle attB^{phi80}::ttrACBSR consumed more tetrathionate than wild type E. coli Nissle as evidenced by increased production of thiosulfate. FIG. 9 further shows that the tetrathionate reducing enzymes (encoded by ttrACB genes) work and that transcription factors, regulating the expression of the ttr operon (encoded by ttrSR genes), perform regulation correctly—in oxic conditions tetrathionate oxidation is less effective. It may therefore be concluded that ttr operon integration with the native promoter region results in the proper functioning of the operon and its activity results in the growth advantage of E. coli Nissle strain in the presence of tetrathionate.

### Example 2—Construction of a Recombinant Probiotic Strain Expressing a Fragment of the GbpA Protein

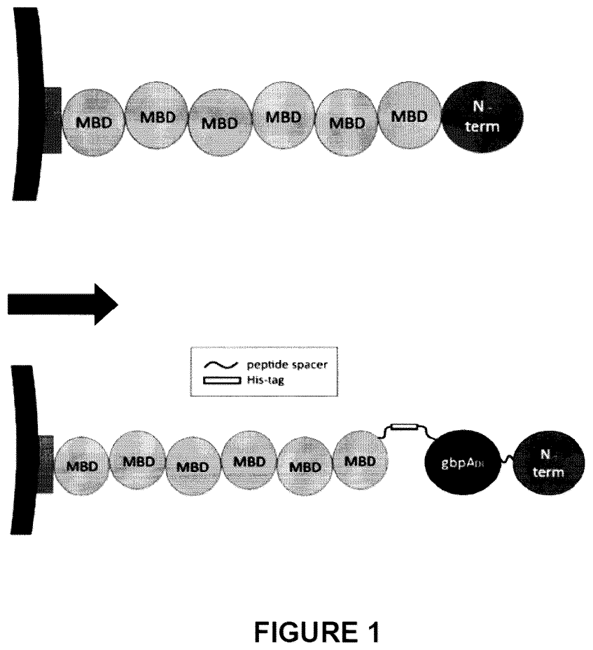

A recombinant probiotic strain of L. reuteri was genetically engineered to express a fragment of the GbpA protein as described herein. In a constructed strain, the isolated fragment of the gbpA gene (SEQ ID NO: 23) encoding the N-terminal (binding) domain is inserted between the mucus-binding domain and the N-terminal domain of MBP from L. reuteri (FIG. 1). Flexible peptide linkers were added between the MBP part of the construction and gbpA part, to make N-terminal domain of GbpA accessible for interaction (see sequences in italics in Table 2). A STREP-TAG 11 was inserted in spacer between N-terminal part of MBP and N-terminal domain of GbpA to make the construction possible to be detected on cell surface with anti-strep-tag 11 antibodies (see underlined sequence in primer 3 in Table 2 (SEQ ID NO: 11)). The final nucleotide sequence of the combined construct containing the insertion of the gbpA fragment within the MBP nucleotide sequence is provided as SEQ ID NO: 30.

### Example 3—Testing the Recombinant Probiotic Strains in the Murine IBD Model

Mice were given probiotic supplementation once daily for three days for testing of E. coli strains and once for testing of L. reuteri strains and then given 3.5% DSS in drinking water for 7 days. All mice were weighed before probiotic administration. All mice across all groups had relatively the same weights and any differences were not significant. Intake of 3.5% DSS drinking water was measured to ensure mice in all groups were being exposed to the same amount of DSS. All mice across all groups had relatively the same water intake and any differences were not significant. Upon sacrifice on day 7, tissues were harvested and examined macroscopically.

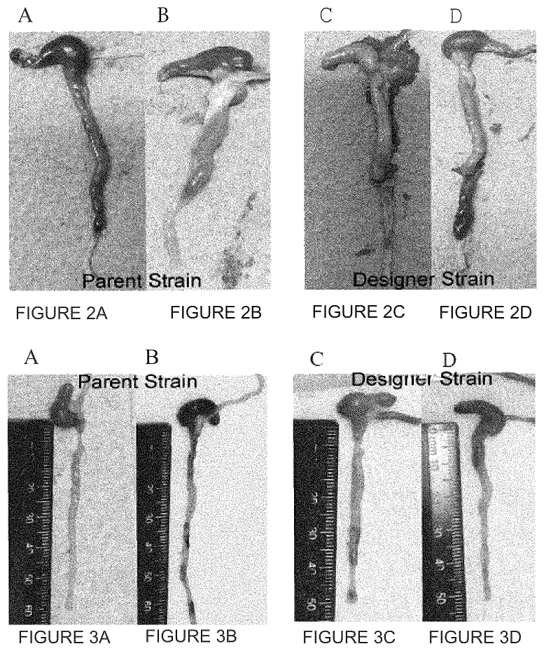

FIGS. 2A-D are a comparison of the ilea, ceca, and colons of mice who were administered either the parent probiotic (E. coli Nissle 1917) or the recombinant probiotic (E. coli Nissle attB^{phi80}::ttrACBSR). FIGS. 3A-D are a comparison of the ilea, ceca, and colons of mice who were administered either the parent probiotic (L. reuteri) or the designer probiotic (L. reuteri::GbpA). In both cases, mice who were administered the parental probiotic strain had very dark loose, rather than formed stool, with blood primarily located in the ceca. Additionally, the colons of these mice also appeared to have loose bloody diarrhoea in lumps still present within the colon and some ulceration mainly near the distal colon (FIGS. 2A, 2B and 3A, 3B). In contrast, mice who were administered the recombinant probiotic strains had less bloody diarrhoea in their ceca and distal colons compared to controls (FIGS. 2C, 2D and 3C, 3D).

As DSS-induced colitis was allowed to progress, mice were given daily clinical scores to score and assess the visual clinical symptoms observed. Mice were scored based on their body movement, rectal bleeding, stool consistency, weight change, and hydration. For body movement, a score of 2 was given for piloerection and a 2 for reduced movement, a score of 3 for hunched posture and a 3 for inactive, and a score of 5 was given for shaking. For rectal bleeding, a score of 1 was given for a positive fecal occult blood test, 2 for blood in the stool, 3 for large amount, and 4 for extensive blood in stool and visible blood at anus. For stool consistency, a score of 1 was given for loose stool, 2 for watery stool, 3 for diarrhoea, and a 4 for no formed stool. For weight, a score of 1 was given for loss of 5-10% of initial weight, a 2 for 10-15%, and weight loss of more than 15% was given a 3. For hydration, a score of 1 was given for slight sunken eyes, 3 for dehydrated eyes, and a 4 for a skin tent. All scores from each category were tallied and a final clinical score per day for each mouse was given during the DSS treatment. Higher clinical scores correlated with increased intestinal inflammation.

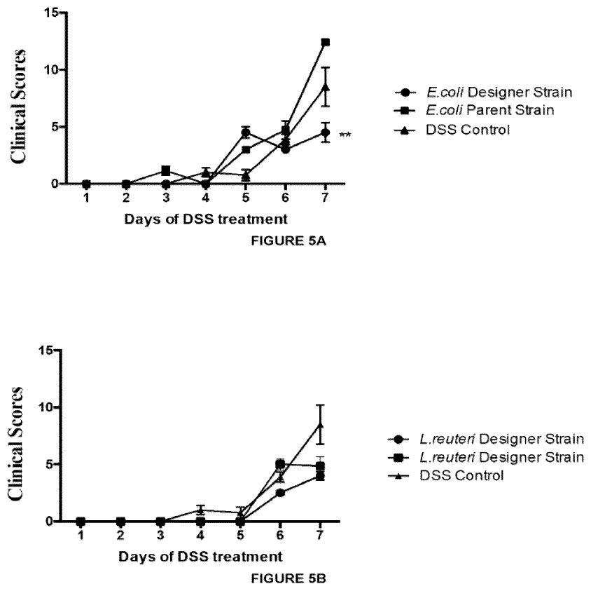

FIGS. 5A and 10B show clinical scores on days 1-7 of DSS treatment for mice who were administered either the parent probiotic (E. coli Nissle 1917) or the recombinant probiotic (E. coli Nissle attB^{phi80}::ttrACBSR). The recombinant probiotic-treated mice displayed reduced clinical scores throughout DSS treatment and shows that administration of the recombinant strain may have improved therapeutic properties over the parent strain. By day 7, the day with the clinically most relevant scores, the parent probiotic reached close to a score of 15 (FIGS. 5A and 10B). The recombinant probiotic had a significantly reduced score of fewer than 5. This further confirms that administration of the designer strain is beneficial over the parent strain.

FIGS. 5B and 11B show the clinical scores on days 1-7 of DSS treatment for mice who were administered either the parent probiotic (L. reuteri) or the recombinant probiotic strain (L. reuteri::GbpA). By day 7, the day with the clinically most relevant scores, shows that the designer strain has a slight advantage over the parent strain in that the clinical score was slightly lower than that of the parent strain (FIGS. 5B and 11B). This again shows that the designer strain does not exert any adverse effect on the host.

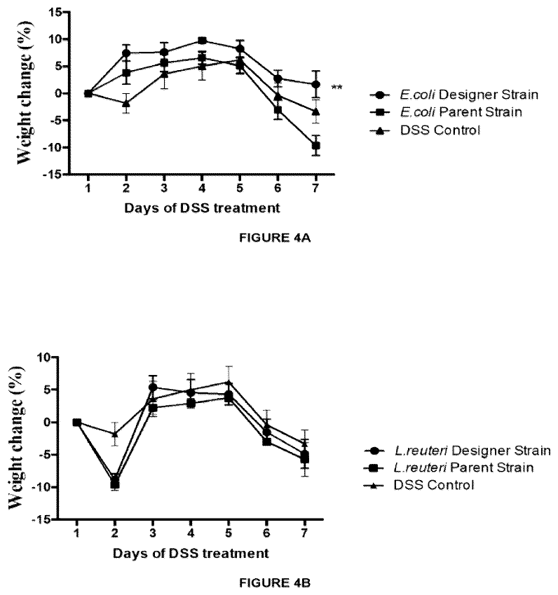

During the 7-day DSS treatment, the body weights of mice who had been administered either the parent probiotic (E. coli Nissle 1917) or the recombinant strain (E. coli Nissle attB^{phi80}::ttrACBSR) were measured. FIGS. 4A and 10A show that mice who were administered the recombinant strain displayed a lower % body weight change overall in comparison to the parent strain. Overall, mice administered the recombinant strain maintained their body weight even though they were challenged with DSS. The recombinant probiotic may therefore provide enhanced protection against colitis. FIGS. 4B and 11A show the body weights of mice who had been administered either the parental probiotic strain (L. reuteri) or the recombinant strain (L. reuteri::GbpA). Both the recombinant and parental strains are shown to have about the same weight change during the course of the DSS treatment. The recombinant strain is shown to have slightly less weight loss over the parent strain. Overall, this shows that the recombinant strain does not have any detrimental effect and is able to provide some protection against the DSS-induced colitis.

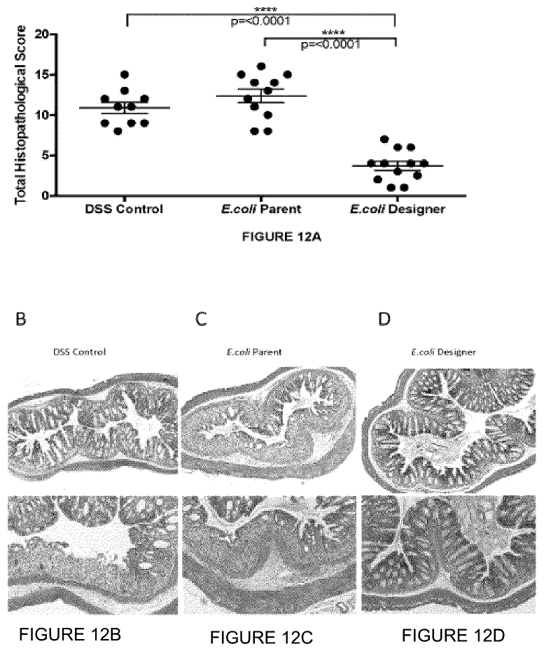

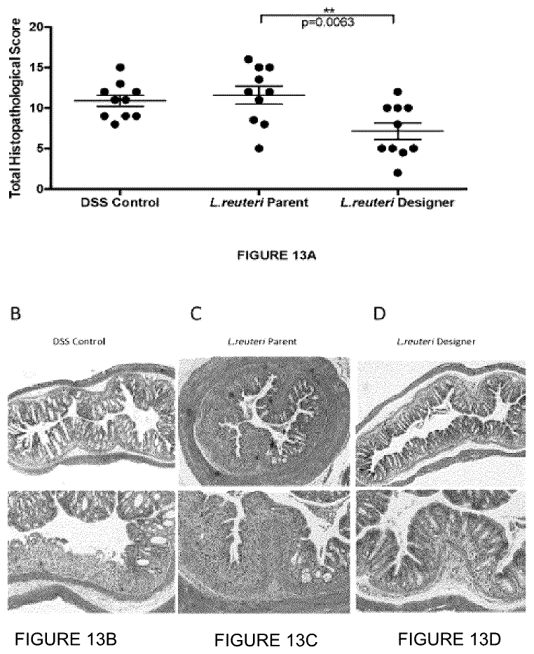

In the second set of trials, to assess histopathological damage, tissue sections were scored based on parameters such as crypt damage, epithelial integrity, goblet cell depletion, and ulceration. A higher histopathological score indicates more inflammation and thus more damage as a result from the DSS-induced colitis. The maximum histopathological score is a score of 16. As shown in FIGS. 12A-D and 13A-D, both the designer strains had lower histopathological scores. This indicates that there is less tissue damage seen in the distal colons of these mice. Both the parent and DSS controls show higher histopathological scores with some mice even reaching a score of 15, indicating severe inflammatory conditions. Histopathological damage in active IBD patients is characterized by inflammation in the colonic mucosa. Inflamed tissue as shown in the DSS control, involves infiltration of immune cells (macrophages, neutrophils, lymphocytes etc.) into the sub-mucosal region, destruction or loss of colonic crypts, ulceration present in the crypts, and depletion of mucosal goblet cells. Based on these histopathological scores, the parent strains resemble the DSS control and thus more of an inflamed damage tissue, whereas the designer strains show lower histopathological scores during the DSS-induced colitis.

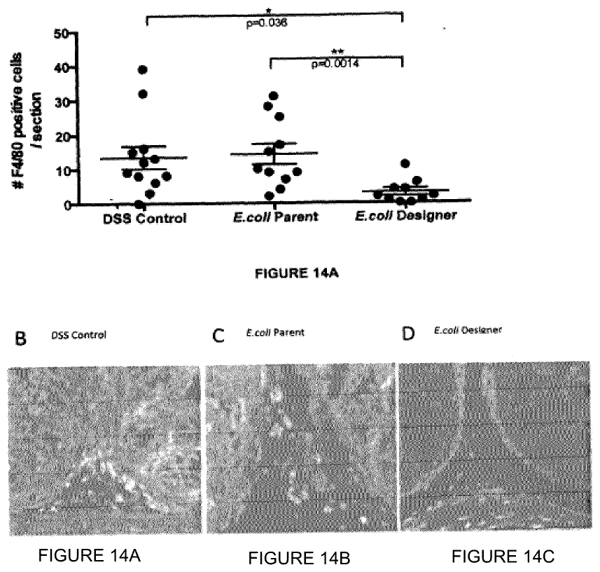

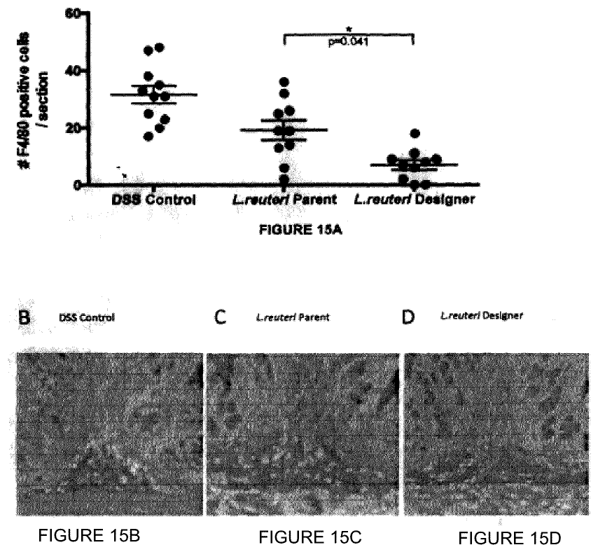

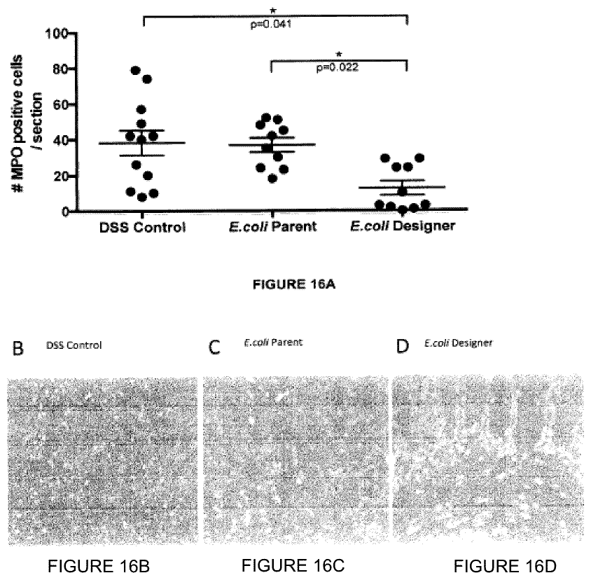

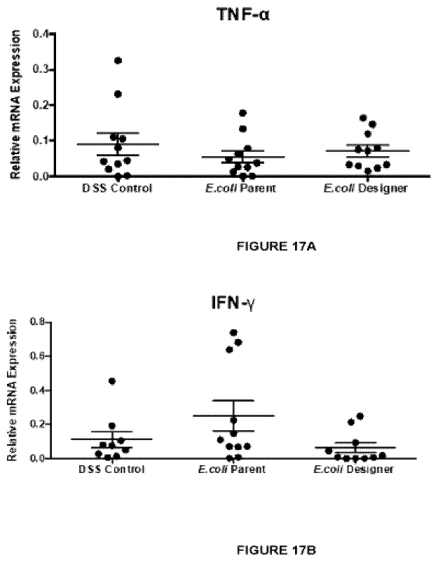

To assess the role of immune cells in the second set of trials, sections of the distal colon were cut onto slides and stained using immunofluorescence. F4/80 marker was used to stain for macrophages. Positive F4/80 cells that co-localized with DAPI stain, for nuclei, were quantified. As shown in FIGS. 14A-D and 15A-D, the designer strains both showed a reduction in the macrophage colonic infiltration. The DSS control and parent strains both show high levels of macrophage cells in the sub-mucosal region. Based on the previous histopathological scores that looked at immune cell infiltration as a parameter, this confirms the previous finding that both the DSS control and parent strains showed increased immune cell infiltration. Although these immune cells are beneficial by acting as the host's defense; excessive recruitment of these cells is seen in inflammatory states. They work in further recruiting more immune cells and signaling molecules like cytokines to the area of inflammation. In a tissue that is undergoing severe inflammation, further recruitment of cells and molecules may be detrimental. It has been shown that in IBD, these immune cells and molecules can result in uncontrolled activation of the immune system and lead to chronic inflammation (Neurath MF Nature Reviews Immunology. 2014; 14:329-342). Further, looking at MPO marker for neutrophils, a similar pattern is observed in FIGS. 16A-D, that the E. coli designer strain has a lower neutrophil infiltration compared to the DSS control and E. coli parent strain.

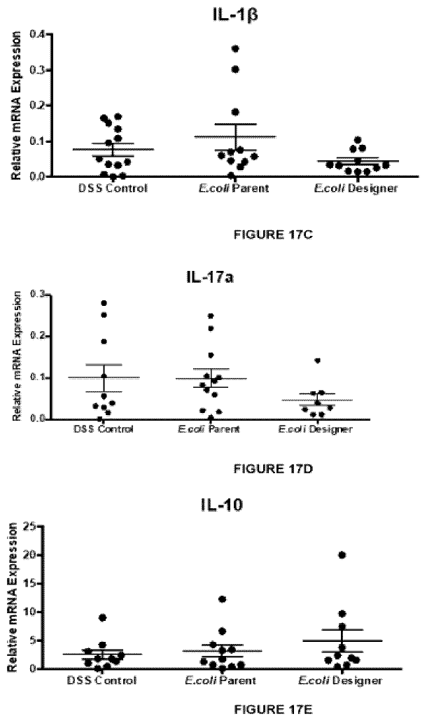

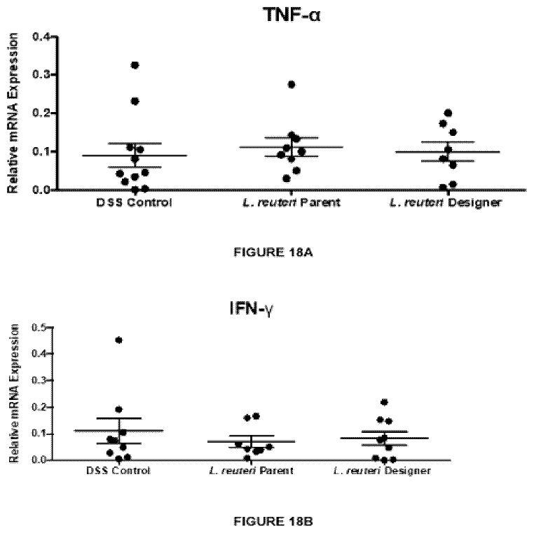

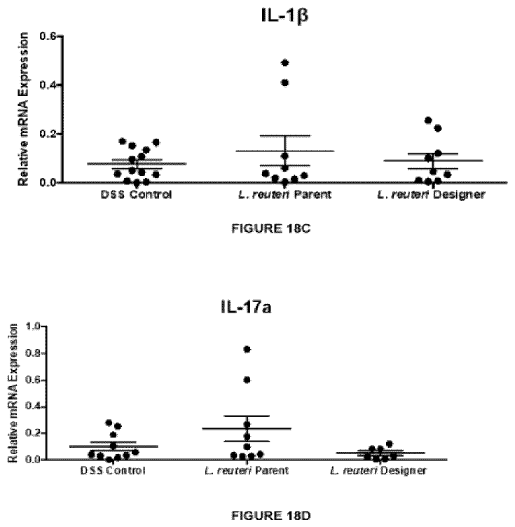

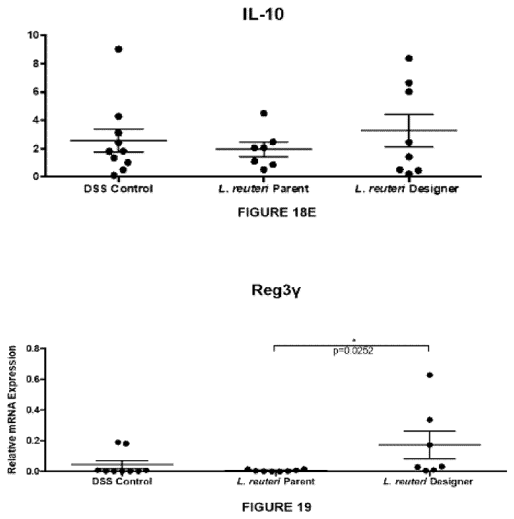

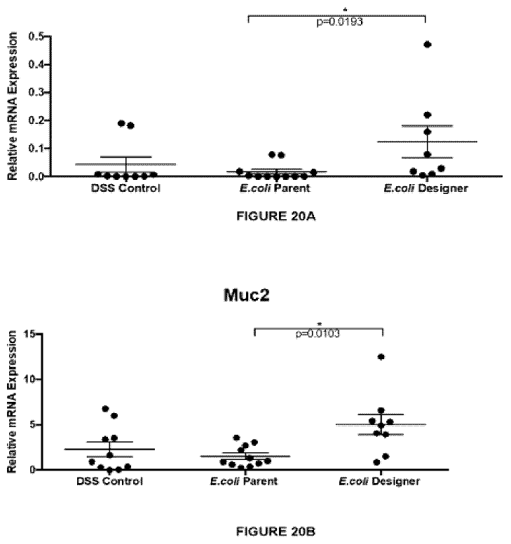

In the second set of trials, to examine if there were any cytokines that were modulated during DSS-induced colitis, pro-inflammatory cytokines were examined. mRNA was synthesized from extracted host RNA in the distal colon. qPCR was used to look at the relative gene expression. As shown in FIGS. 17A-E and 18A-E, there was an overall pattern in that the pro-inflammatory cytokine gene expression (TNF-α, IFN-γ, IL-1β, and IL-17a) in mice administered the designer strains was lower than the DSS control animals and mice administered the parent strain. Lower expression of these pro-inflammatory cytokines can help reduce some of the uncontrolled activation seen during inflammation and thus can help control some of the symptoms seen during DSS-induced colitis. In contrast, the expression of the anti-inflammatory IL-10 cytokine was shown to be increased in mice administered the designer strains as compared to the parent strain and the DSS control (FIGS. 17E and 18E) This shows that the designer strains are much more protective during inflammation compared to the parent strains. To further look at protective responses, the gene expression of Reg3 and Mucin2 was examined. As shown in FIGS. 19 and 20A-B, the gene expression of these was up-regulated in mice administered the designer probiotic strains as compared to the parent strains and the DSS control. Reg3γ is an anti-microbial peptide that targets gram-positive bacteria by binding to the peptidoglycan layer (Ratsimandresy R A, et al. Cellular & Molecular Immunology. 2017; 14:127-142). The higher expression of this peptide can help in controlling some of the opportunistic bacteria that can populate as a result of the damaged epithelial layer. Following the administration of the E. coli designer strain, Muc2 gene expression is higher in mice as compared to animals that received the parent strain. Muc2 is a colonic secretory mucin that is synthesized by goblet cells (Bergstrom K S et al. PLOS Pathogens. 2010; 13(6)). It makes up the mucus layer found in the gut epithelial. Increased expression of this would be beneficial in a tissue undergoing inflammation. With the gene expression of these protective responses, the designer strains are found to be more protective than the parent strains.

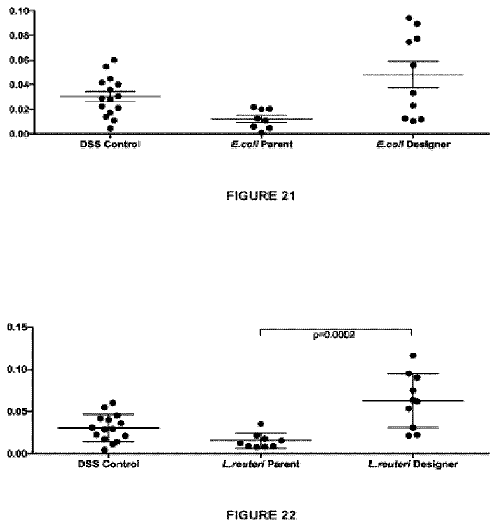

To further explore protective responses, the production of short chain fatty acids (SCFAs) was examined. The by-products of fermentation of indigestible dietary residues result in metabolites such as short chain fatty acids (SCFAs). SCFAs have many roles such as nutrients for colonic epithelium, mediating intercellular pH, cell volume, ion transport, and regulation of proliferation, differentiation, and gene expression. Butyric acid not only acts as fuel for colonic epithelial cells but it also regulates cell proliferation and differentiation. Butyric acid is preferred over propionate and acetate in colonocyte metabolism, where butyrate oxidation makes up 70% of the oxygen consumed by colonic tissue (Morrison D J and Preston T. Gut Microbes. 2016; 7(3):189-200). Since, butyric acid is an important regulator of colonic proliferation, increased amounts are beneficial during inflammation. SCFAs were examined using gas chromatography. As shown in FIGS. 21 and 22, butyric acid was found to be more abundant in animals that received the designer stains compared to the parent stains and DSS control. Acetic acid and propionic acid showed no significance differences. This further shows that the designer strains are more protective compared to the parent strains.

### Example 4—Pro-Inflammatory Cytokine Expression

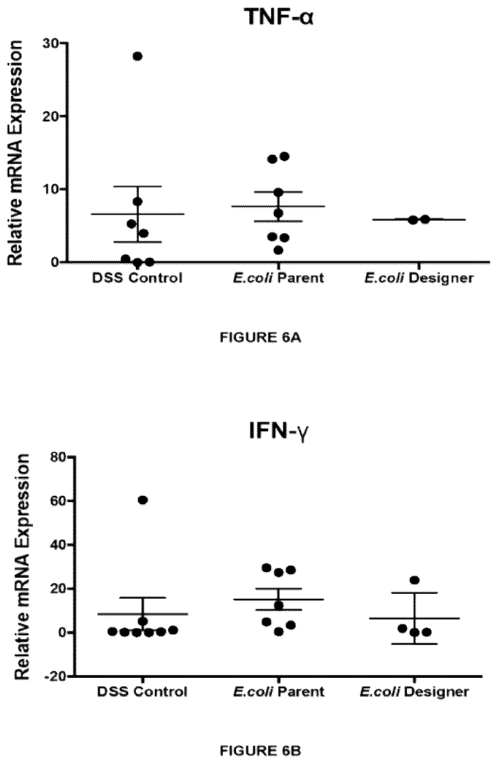

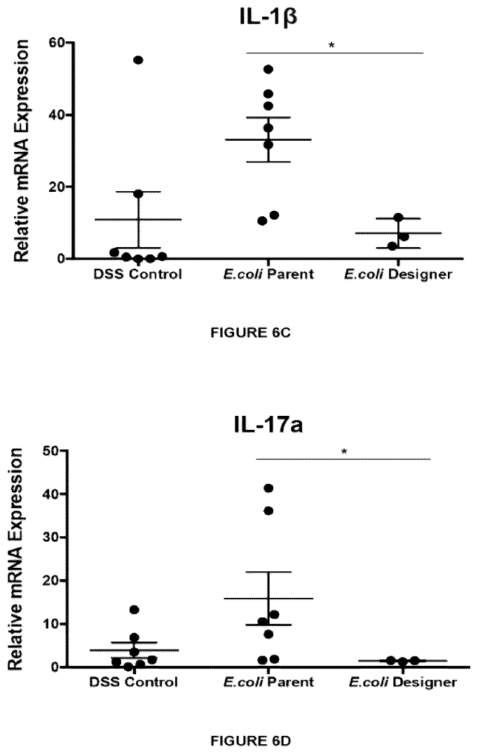

In order to investigate the protection seen in the recombinant strain (E. coli Nissle attB^{phi80}::ttrACBSR), we examined whether there were any cytokines that were modulated during DSS-induced colitis. At day 7 of the DSS treatment, mice were sacrificed and inflammatory cytokine levels in colonic tissues were assessed. FIGS. 6A-D show cytokines levels in colonic tissues of mice who received either the parent probiotic (E. coli Nissle 1917) or the recombinant probiotic (E. coli Nissle attB^{phi80}::ttrACBSR). Overall, mice who received the recombinant strain showed a trend towards lower levels of cytokine expression in comparison to those who received the parent strain. FIGS. 6A-D show the parent strain is associated with higher levels of pro-inflammatory cytokine expression, indicating that the designer probiotic may have an improved protective effect during colitis, compared to the parent probiotic strain. The most drastic and signficant differences were seen with the pro-inflammatory cytokines interleukin-1 beta (IL-1β) and interleukin-17a (IL-17a). IL-1β is a mediator of inflammatory responses that are involved in cell proliferation, differentiation, and apoptosis. IL-17a is a signaling molecule secreted mainly by T-helper cells and may be a mediator of inflammatory responses. It induces the activation of certain genes that are associated with inflammation. It further stimulates pro-inflammatory responses, including those induced by IL-1β. The lowered expression of these pro-inflammatory responses, especially during colitis, help to reduce the inflammation and this would be beneficial in controlling the symptoms seen. The parent strain is seen to have highly elevated expression of these pro-inflammatory cytokines, indicating that there are high levels of inflammation undergoing. Therefore, the designer parent is seen to be much more protective during colitis compared to the parent strain probiotic.

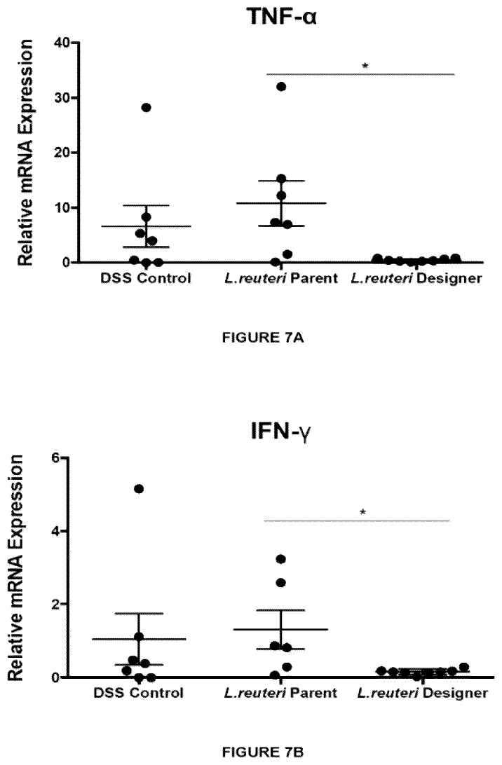

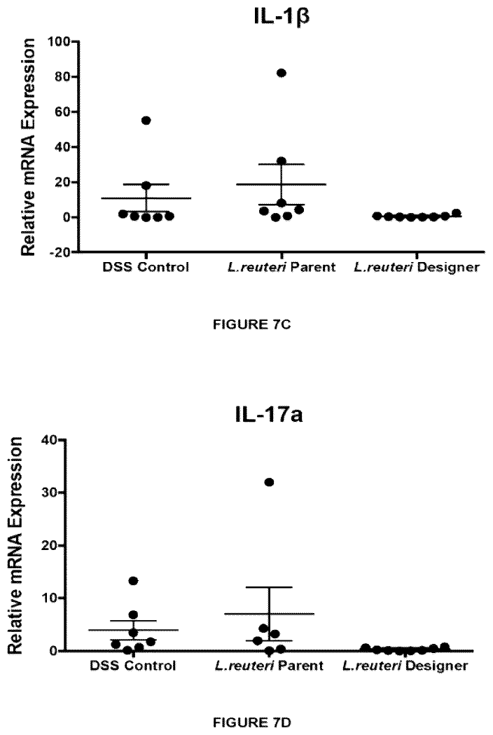

FIG. 7A-D show cytokine profiles from colonic tissues of mice treated with either the parental strain (L. reuteri) or the recombinant strain (L. reuteri::GbpA). Most of the cytokines examined showed a trend in which the recombinant strain had lower expression levels. The most drastic and significant differences were seen with the pro-inflammatory cytokines tumor necrosis factor alpha (TNF-α) and interferon gamma (IFN-γ). TNF-α is a signaling molecule that plays a role in the activation of further inflammatory responses. IFN-γ is another typical pro-inflammatory cytokine that further stimulates more immune cells, such as natural killer (NK) and T cells. Such pro-inflammatory cytokines are elevated during conditions like IBD and lead to tissue damage from increased inflammation. Therefore, the lowered expression of these pro-inflammatory responses, especially during colitis, help to reduce the inflammation and this would be beneficial in controlling the symptoms seen. The parent strain is seen to have highly elevated expression of these pro-inflammatory cytokines, indicating that there are high levels of inflammation undergoing. Thus, the recombinant strain is seen to be much more protective during colitis compared to the parent strain.

### Example 5—Muc2^{-/-} Spontaneous Colitis with E. coli

The designer strains were shown to provide protection during DSS-induced colitis; therefore, we examined another model of murine colitis, Muc2^{-/-} spontaneous colitis. Muc2^{-/-} mice develop spontaneous colitis, which is characterized by hyperplasia, crypt abscesses, immune cell infiltration, and sub-mucosal edema (Morampudi V. et al. Mucosal Immunology. 2016:1-16). These all represent clinical features of active ulcerative colitis. Mucin 2 is the prominent mucin synthesized in the colon and therefore a defective mucus barrier in animal models allows bacterial contact with the intestinal epithelium (Morampudi V. et al. Mucosal Immunology. 2016:1-16). This results in spontaneous colitis since a defective mucus barrier is seen in ulcerative colitis. Muc2^{-/-} mice can develop rectal prolapse and this would indicate sever inflammation and human endpoint for the mice. Muc2^{-/-} mice bred on a C57BL/6 background were administered either E. coli parent stain or designer strain. These animals were split into two cohorts at 3 months of age and at 4 months of age to look at the disease progression with age. Mice were orally gavaged a dose of probiotics once weekly for 4 consecutive weeks. The clinical scores and weight change of these animals was monitored weekly throughout the entire Muc2^{-/-} spontaneous colitis.

The rate of rectal prolapse is summarized in Table 4. The Muc2 control had a 5% rectal prolapse rate, parent strain had a 20% rectal prolapse rate and the designer strain had a 0% rectal prolapse rate. The E. coli designer strain had no rectal prolapses in all cohorts of 3 and 4 months of age mice, indicating that the E. coli designer strain is providing protection.

TABLE 4
Rate of rectal prolapse in 3 month and 4-month old Muc2^{-/-} mice

| Treatment | Muc2^{-/-} | Parent | Designer |
|---|---|---|---|
| Groups | Control | Strain | Strain |
| Number of rectal | 1/19 (5%) | 3/15 (20%) | 0/20 (0%) |
| prolapses | | | |

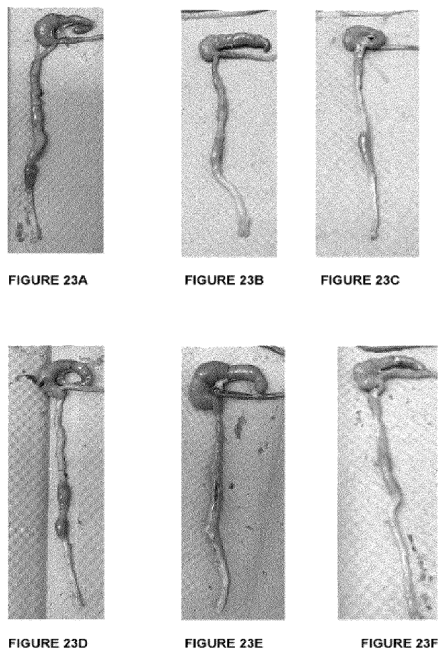

Macroscopic images of the distal colon and ceca were taken and, in FIGS. 23A-F, show that in both 3-month (FIGS. 23A-C) and 4-month (FIGS. 23D-F) old Muc2^{-/-} mice, the Muc2^{-/-} control and parent strain groups showed more swollen distal colons compared to the designer strain groups. The ceca of the Muc2^{-/-} control and parent strain mice are more enlarged in size in comparison to the designer strain. This indicates that the administration of the designer strain resulted in less swollen inflamed tissues.

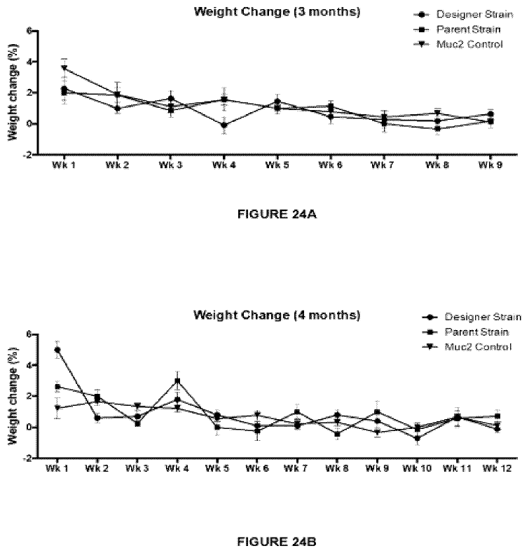

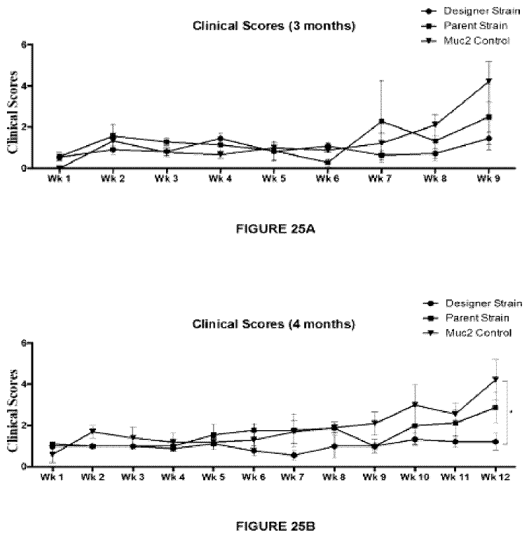

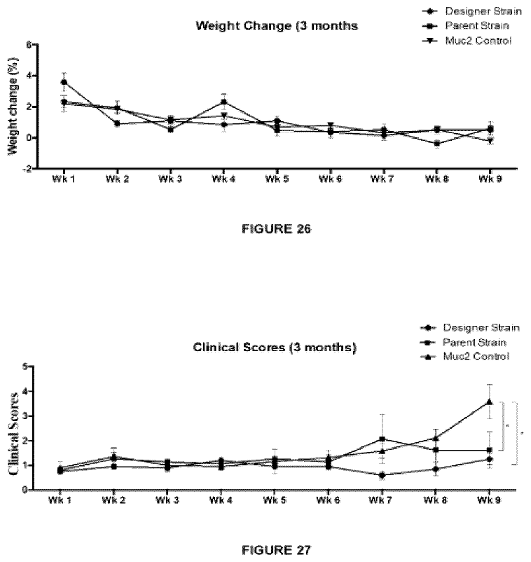

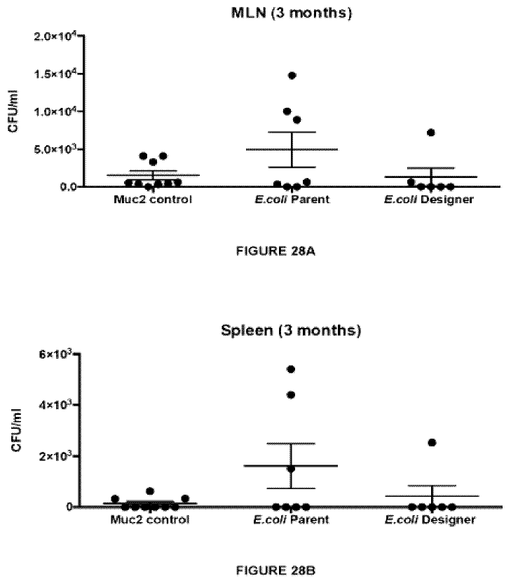

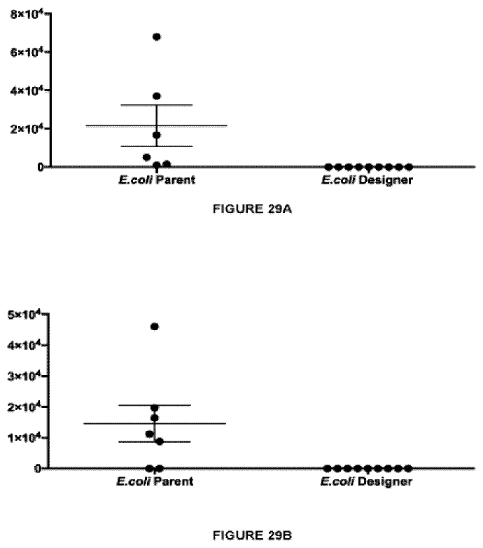

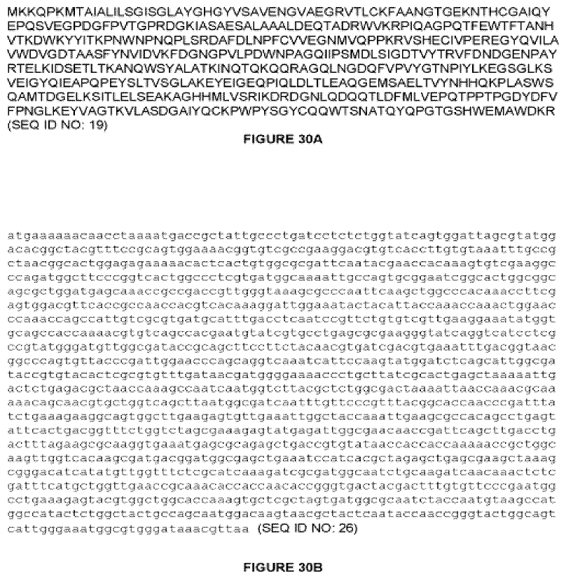

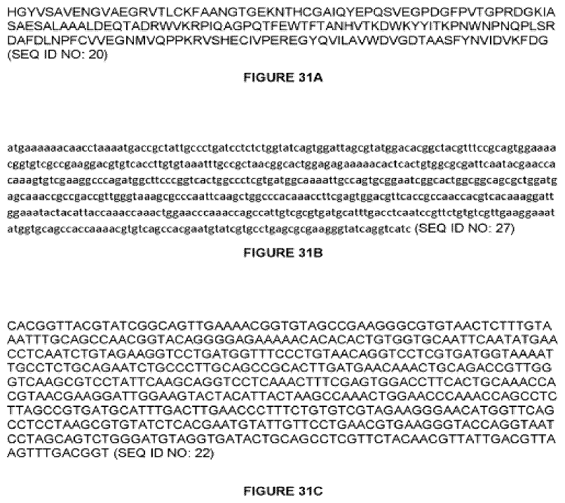

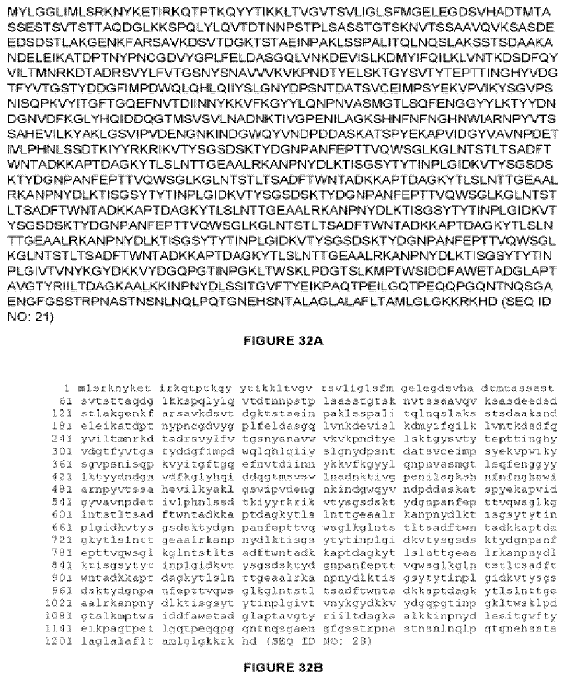

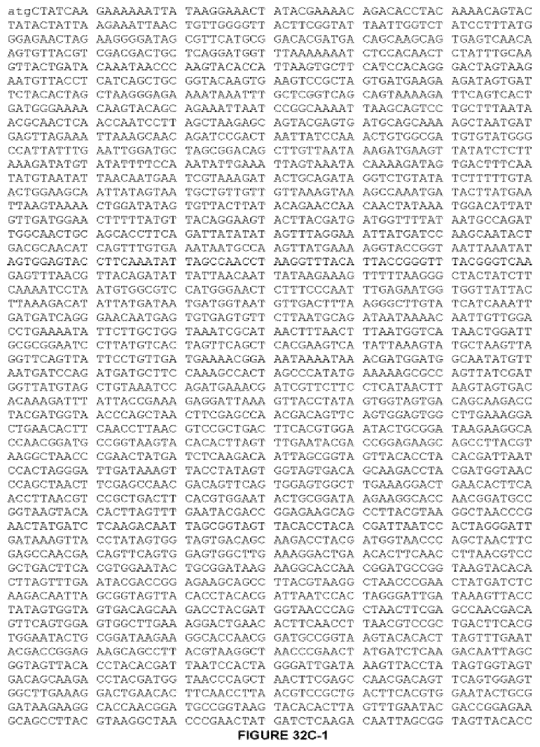

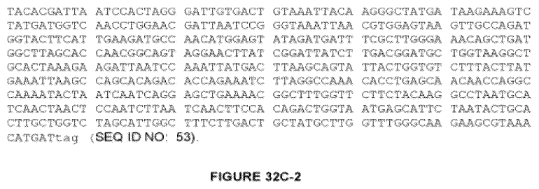

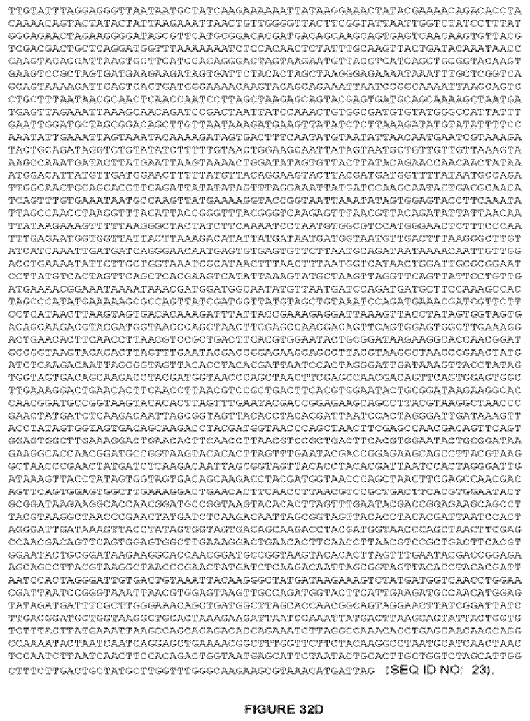

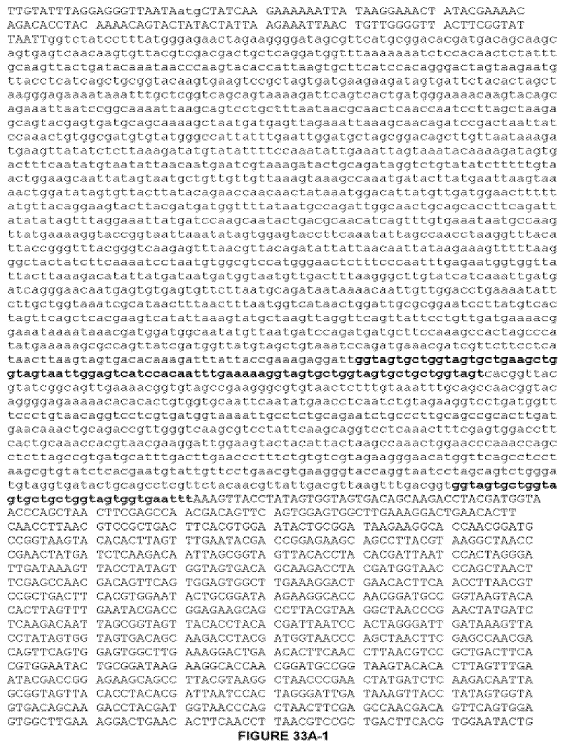

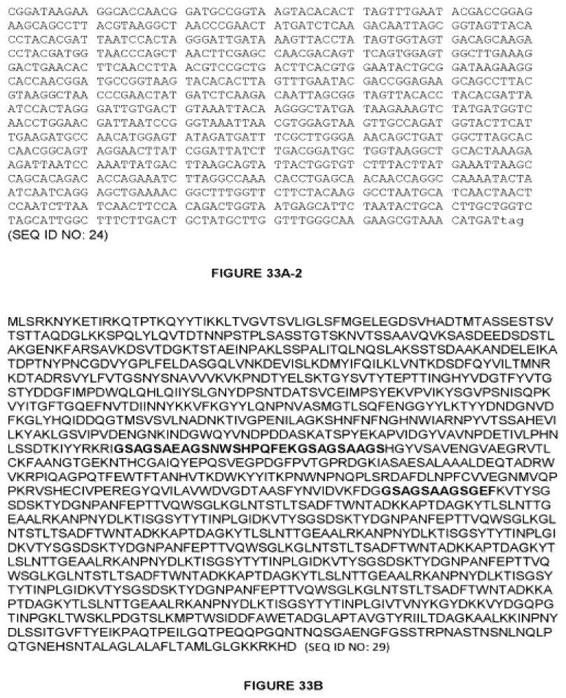

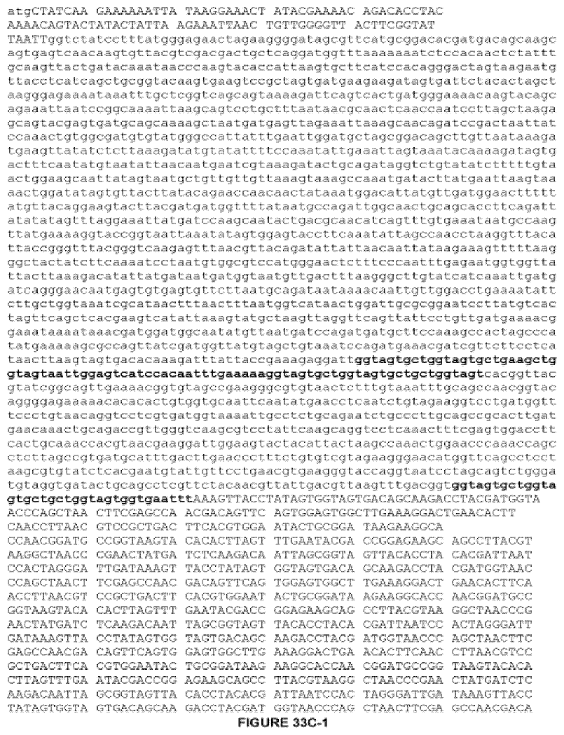

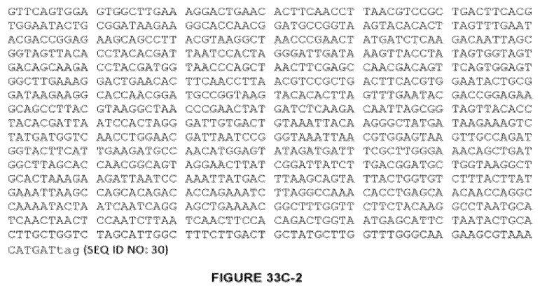

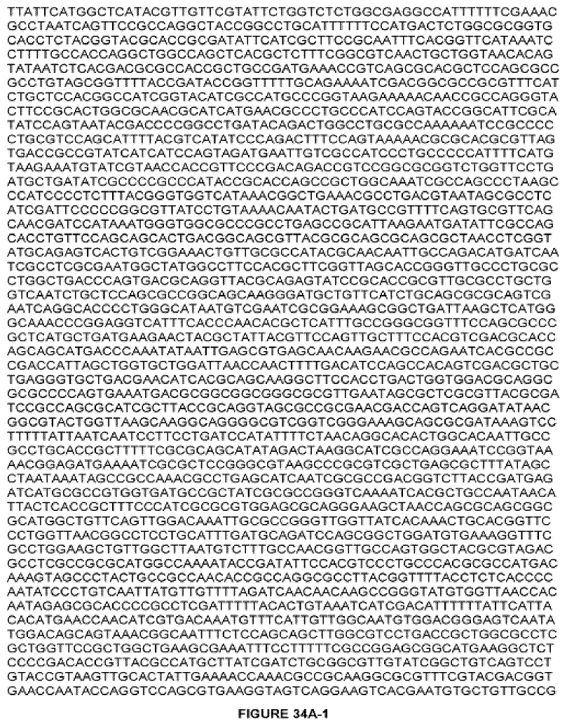

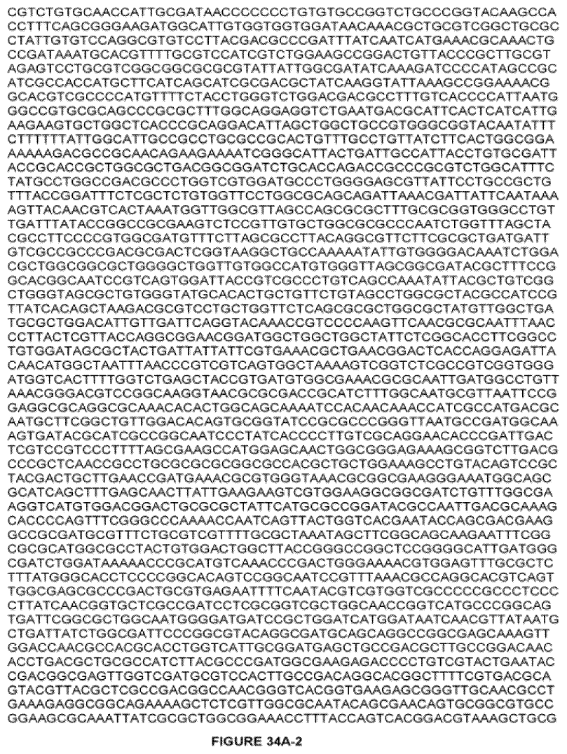

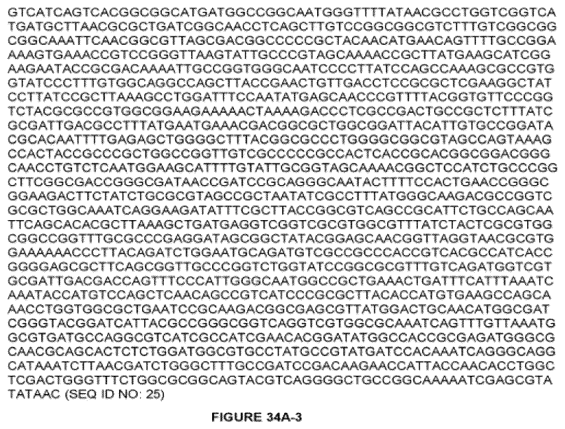

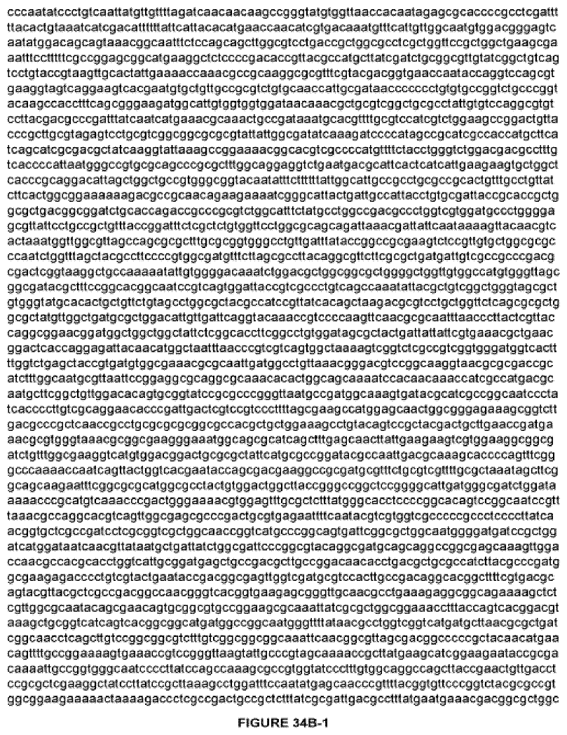

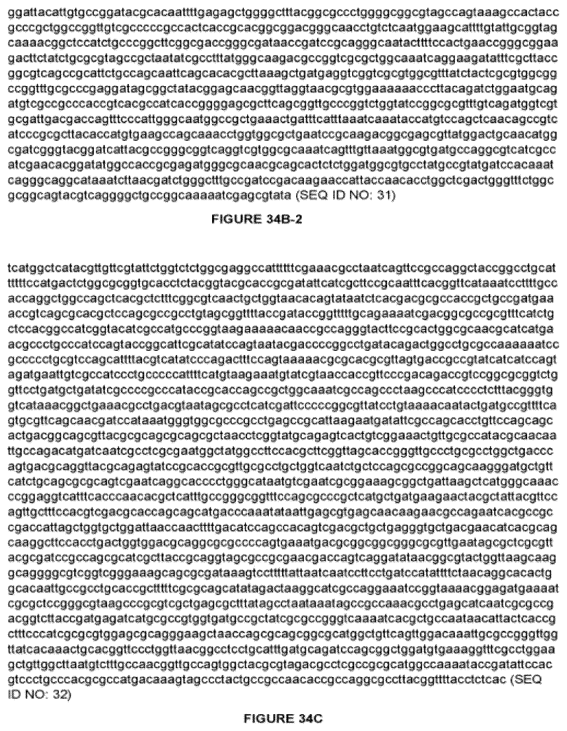

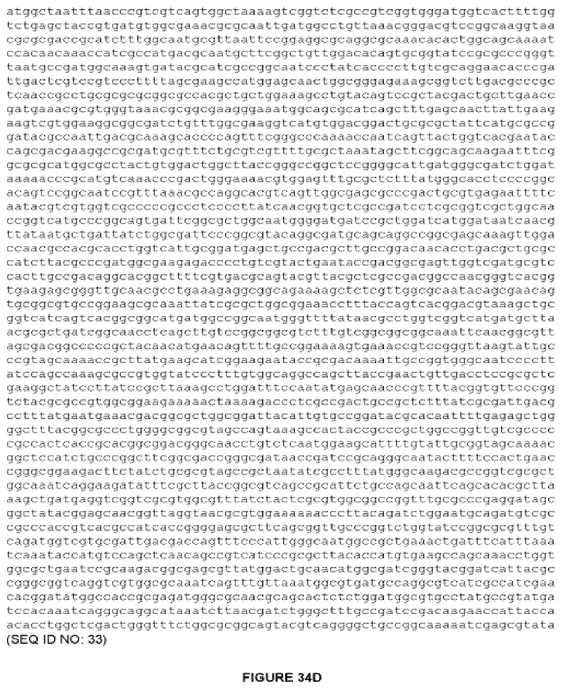

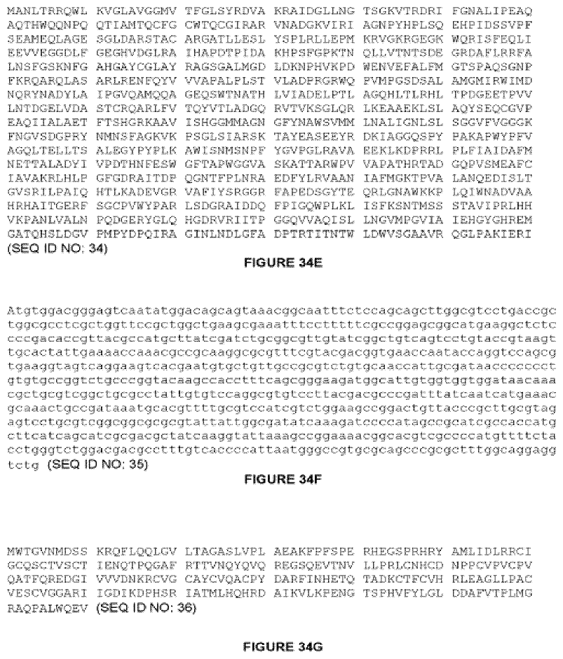

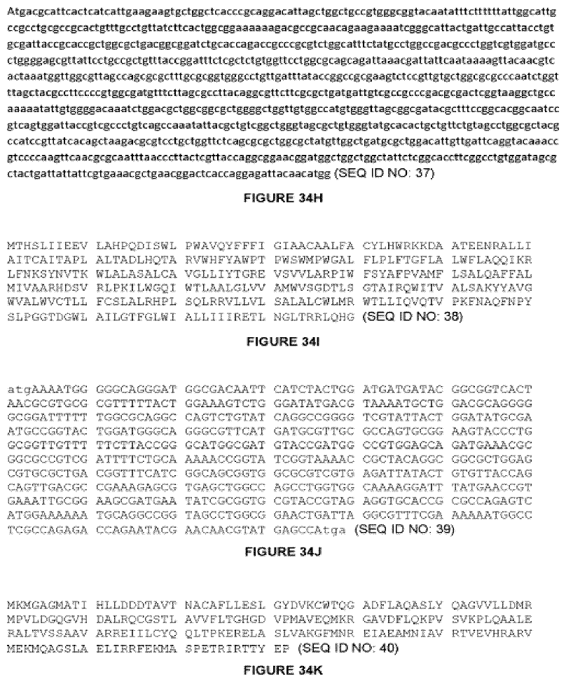

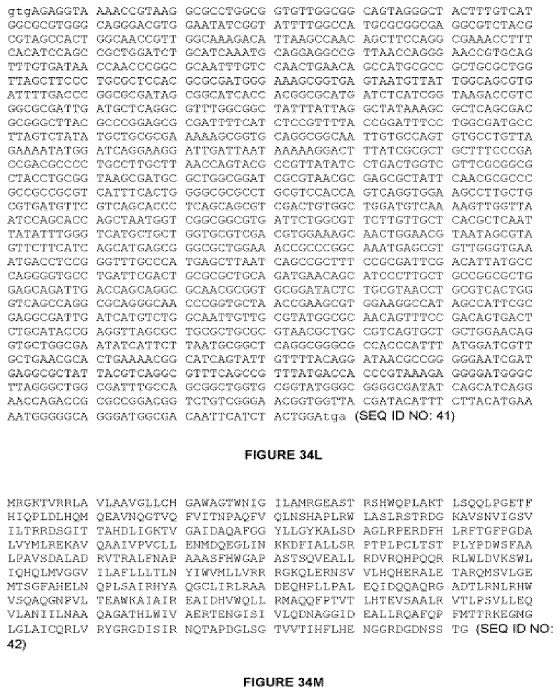

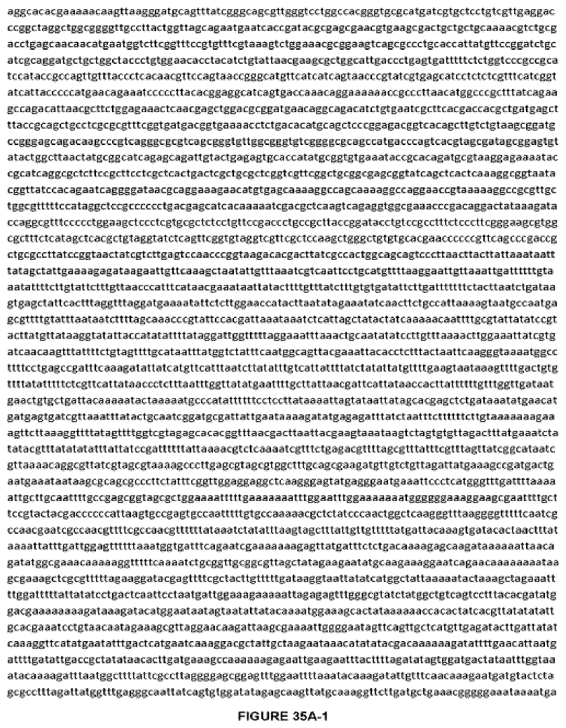

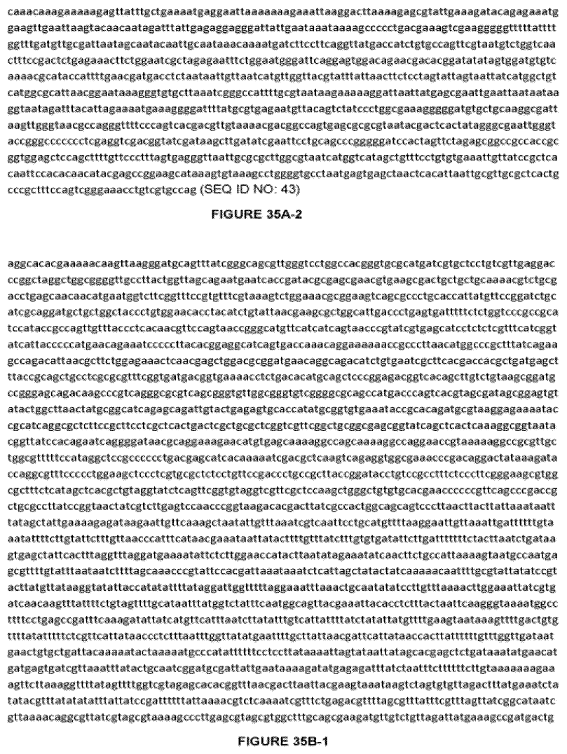

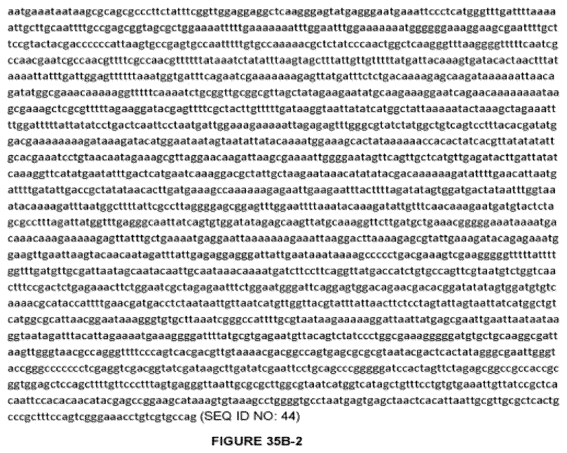

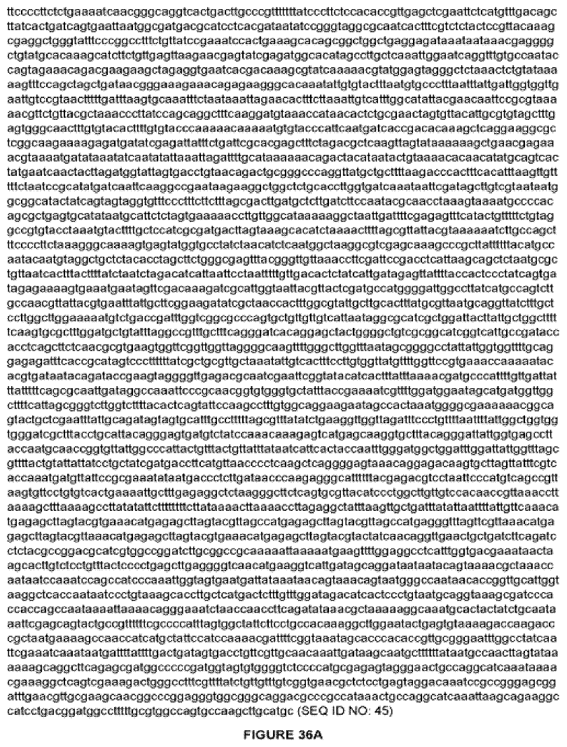

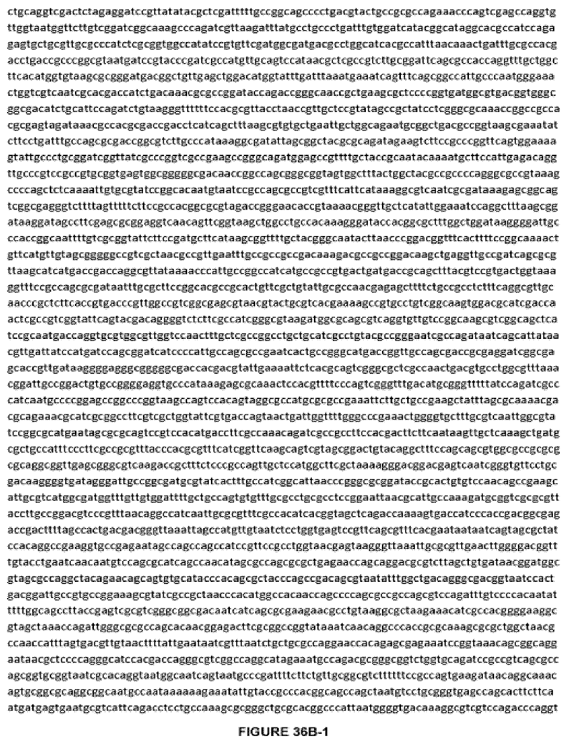

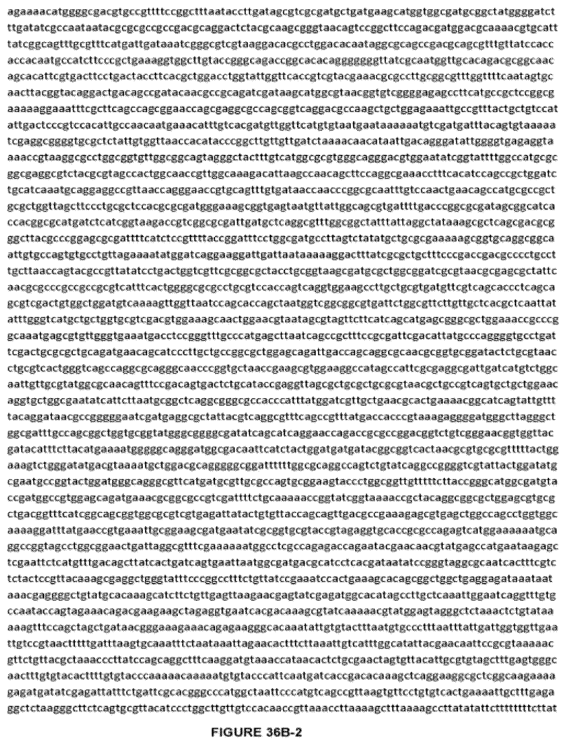

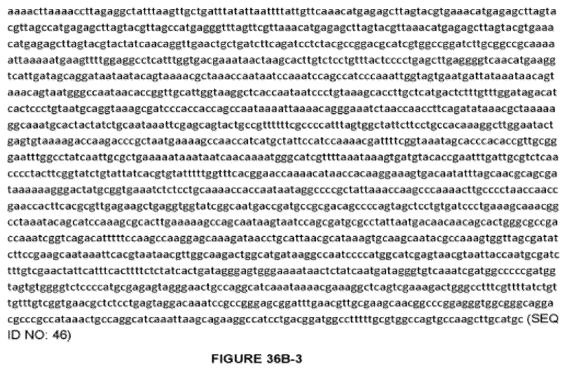

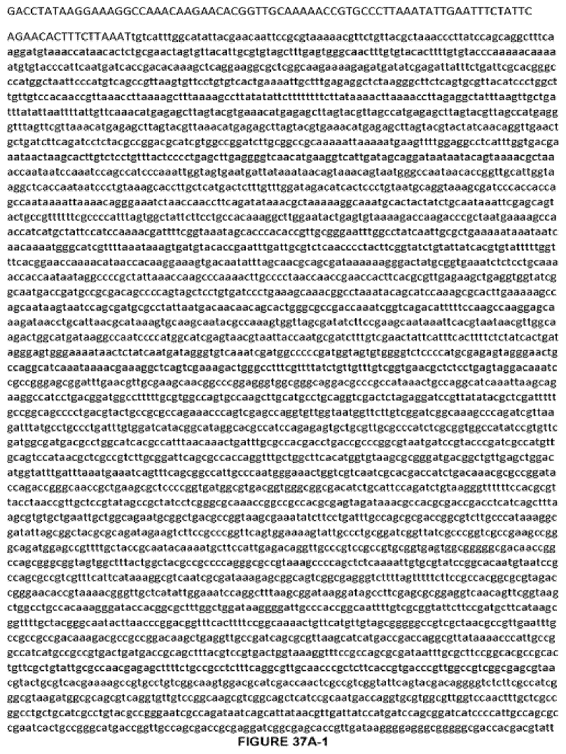

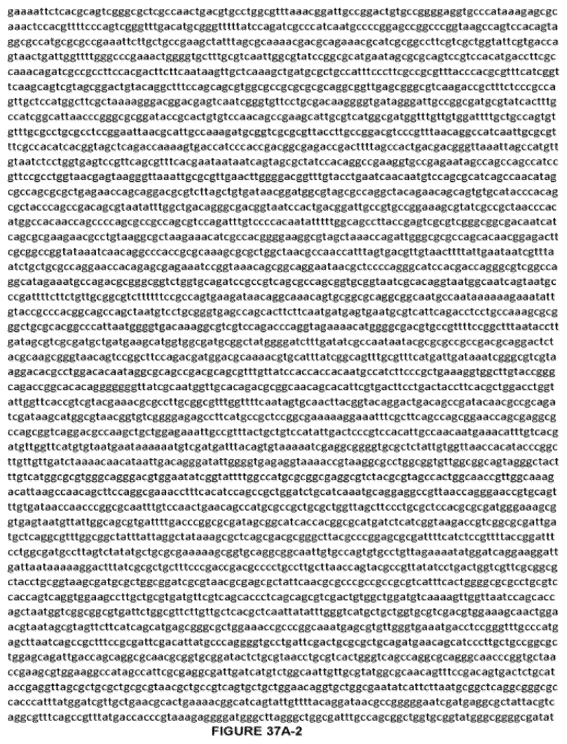

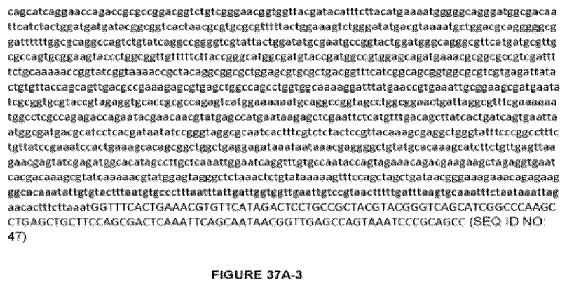

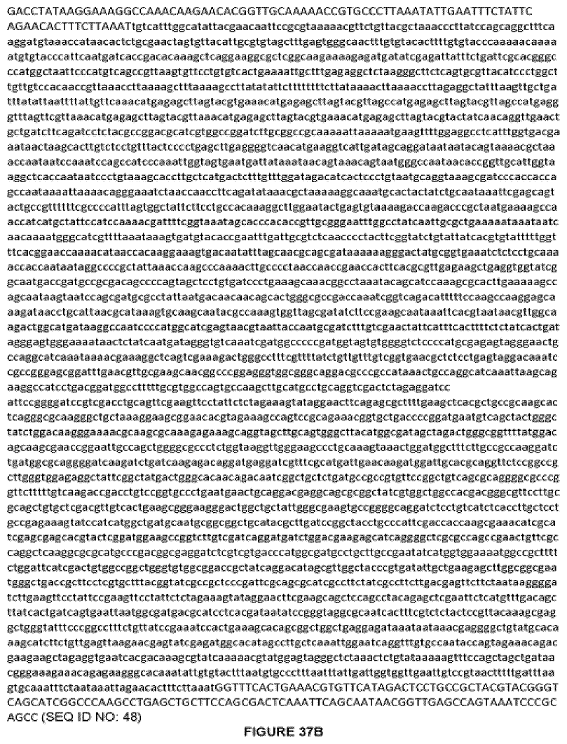

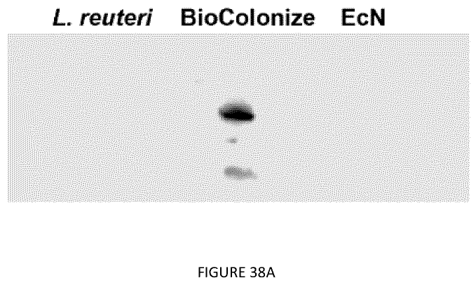

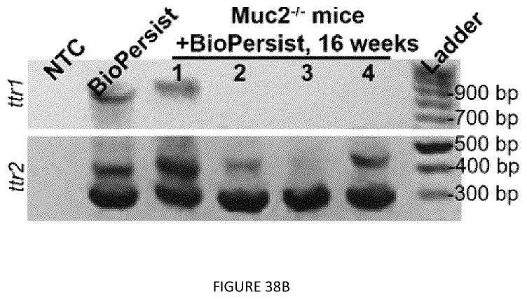

The body weight changes and clinical scores of the Muc2^{-/-} mice at 3 months and 4 months of age are shown in FIGS. 24A-B, 25A-B, 26 and 27. Body weight changes do not show significant changes but when graphing all mice at 3 months of age, the designer strain group shows a slight difference with less body weight loss compared to the Muc2^{-/-} control mice shown in FIG. 26. For clinical scores, parameters such as body movement, stool consistency, weight change, and hydration were examined. Shown in FIGS. 25A-B and 27, at 3 months and 4 months of age, the designer stain group shows lower clinical scores compared to the Muc2^{-/-} control and a slight advantage over the parent strain group. This indicated that the administration of the designer strain in Muc2^{-/-} mice show less clinical symptoms during the disease progression.

To examine if there was a systemic infection, the MLN and spleen was homogenized and then plated on 1.8% LB agar plates to obtain colony counts. Bacterial translocation would result in the passage of viable bacteria from the digestive tract into other body sites that normally would not have bacteria present. Such sites like the MLN and spleen can be used as indicators of bacterial translocation and high amounts of this translocation could result in sepsis. Bacterial translocation indicates that there is dysregulation in either the epithelial layer or the host immune defenses or a combination of both. As shown in FIGS. 28A-B and 29A-B, there are lower colony forming units (CFU) per mL of homogenate plated in the designer strain animal group compared to the parent strain and Muc2^{-/-} control animal groups at both 3 and 4 months of age. This indicates that there can be a leaky gut present in the Muc2^{-/-} mice administered the parent strain and the Muc2^{-/-} control mice that is allowing the passage of bacteria into extra-intestinal sites. Thus, with the rectal prolapse rate at 0%, lower clinical scores, and lower bacterial CFU/mL counts the administration of the E. coli designer strain is shown to be more protective during Muc2^{-/-} spontaneous colitis compared to the parent strain and the no probiotic Muc2^{-/-} control.

The results indicate that the E. coli and L. reuteri designer probiotics were more efficacious during DSS-induced colitis compared to the unmodified parent strains. Macroscopic examination revealed that the modified designer probiotics had less bloody and loose stool in the colon and cecum compared to the unmodified parent strains. The designer probiotics also lost less body weight and had lower clinical scores during the DSS-induced colitis period. The unmodified parent DSS groups lost more of their initial starting body weight and had high clinical scores, indicating humane endpoint in some mice. Histopathologically, the designer strains showed lower histopathological scoring compared to the parent and control groups, as well as fewer gene expression levels of pro-inflammatory markers such as TNF-α, IFN-γ, IL-1β, and IL-17a. In contrast, the unmodified parent strains showed elevated expression of many pro-inflammatory markers, indicating no improvement during IBD. The designer strains also showed a trend of an increase in the expression of the anti-inflammatory cytokine IL-10. The designer strains further showed lower counts of macrophage cell infiltration and the E. coli designer strain showed lower counts of neutrophil infiltration, indicating that these designer strains have less damage in the colonic tissue. In terms of protective responses, both the designer strains had an increase of expression in Reg3γ and increased production of the bacterial metabolite butyric acid. The E. coli designer strain further had an increase in Muc2 gene expression. Looking at the Muc2^{-/-} spontaneous colitis model with the E. coli designer strain, there were no rectal prolapses shown compared to the 5% and 20% for the Muc2^{-/-} control and parent strain, respectively. There were also lower clinical scores and lower incidence of bacterial translocation in mice that were administered the E. coli designer strain as compared to the Muc2 control^{-/-} and mice administered the E. coli parent strain. Overall, this shows that the E. coli and L. reuteri are more protective during DSS-induced colitis. In addition, in the tested E. coli designer strain in Muc2^{-/-} spontaneous colitis, the E. coli is more protective compared to its parent strain.

### Example 6—Persistence and Colonization of Designer Strains

To understand where the recombinant strains preferentially occupy a niche in the IBD gut, we examined their presence close to the epithelial surface in the ileum, cecum, and colon of colitis-prone Muc2^{-/-} mice over time. After a single dose of either recombinant strain, orally administered to Muc2^{-/-} prior to spontaneous colitis, we found both occupied the cecum and colon throughout the 10-day experiment.

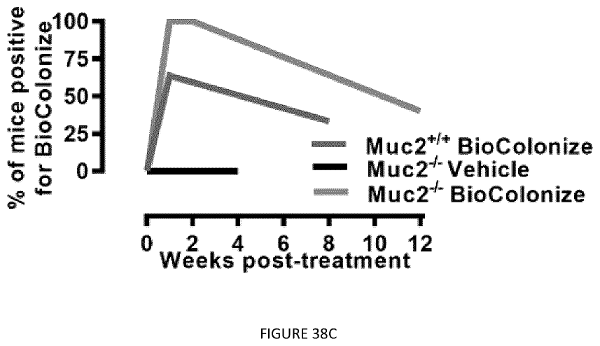

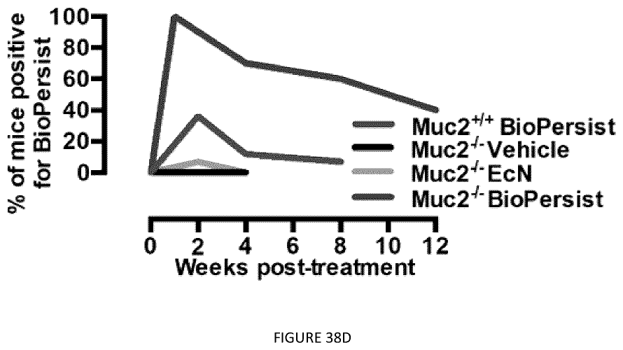

To understand if designer strain presence in the large intestine could be detected even when the severe colitis phenotype is observed, we looked at Muc2^{-/-} mice from 0-12 weeks post-gavage. As shown in FIGS. 38A and 38C the presence of GbpA, was detected in 75% of Muc2^{-/-} mice and 60% of C57BL/6 WT mice after 12 weeks. As shown in FIG. 38B, the ttr operon was detected in >45% of colitis-prone Muc2^{-/-} mice after 12 weeks post-gavage, in contrast to the parent strain, which was only present in <5% of Muc2^{-/-} mice disappearing completely by 4 weeks, as shown in FIG. 38D. This data supports our hypothesis that the ttr operon helps provide a survival advantage in the inflamed colon. Unlike the Muc2^{-/-} colitis-prone mice, healthy WT mice harbored little to no recombinant strain expressing ttr, as shown in FIG. 38D. Therefore, the ttr operon promotes persistence only during colitis and loses its ability to occupy the healthy gut because the ttr operon provides a selective survival advantage only during inflammation in the gut. This alleviates the potential for the designer strain expressing ttr to over-colonize the gut, thereby supporting a natural “kill” switch. Indeed, we show that the designer strain expressing ttr does not overtake the gut microbiome following intervention. In addition, in C57BL/6 mice, there were no observable histological, physiologic, or immunologic changes for either designer strain, suggesting they are safe to consume. Overall, the designer strain expressing GbpA does colonize the large intestine, competing even with a healthy native microbiome while the designer strain expressing ttr can occupy a niche in the large intestine and has a survival advantage persisting only during colitis.

### Example 7—Designer Strain Expressing Ttr is More Effective than Front-Line Therapy 5-ASA in DSS-Induced Colitis

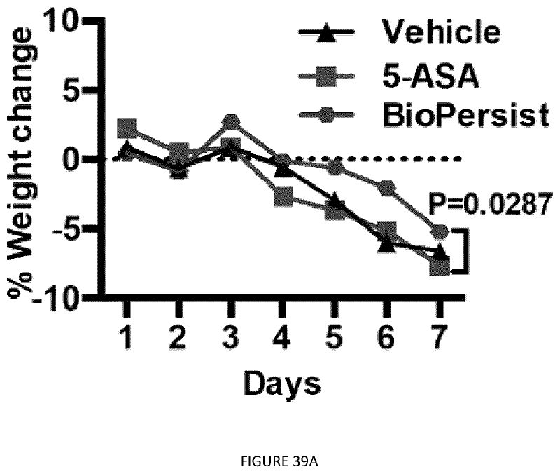

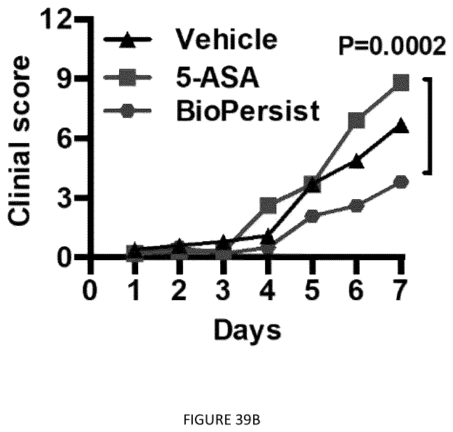

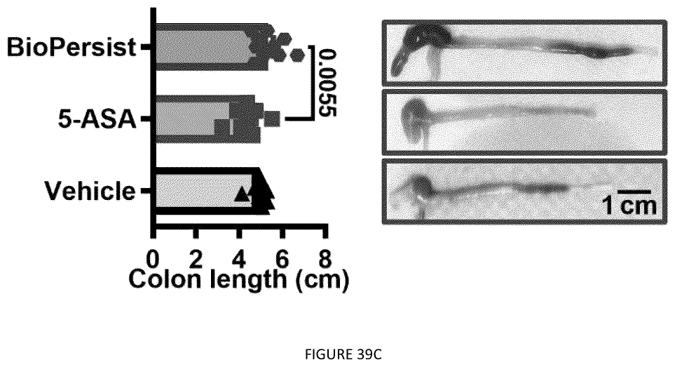

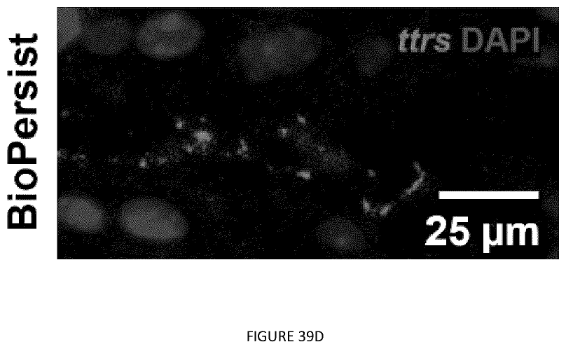

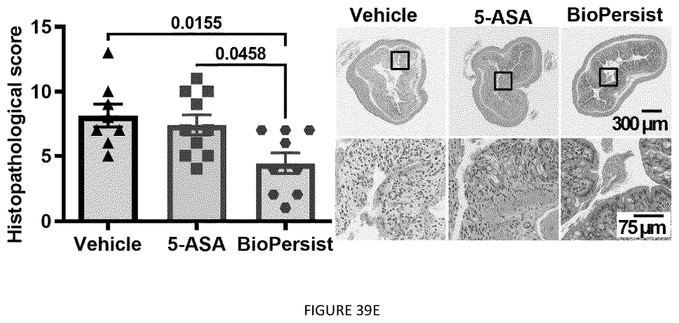

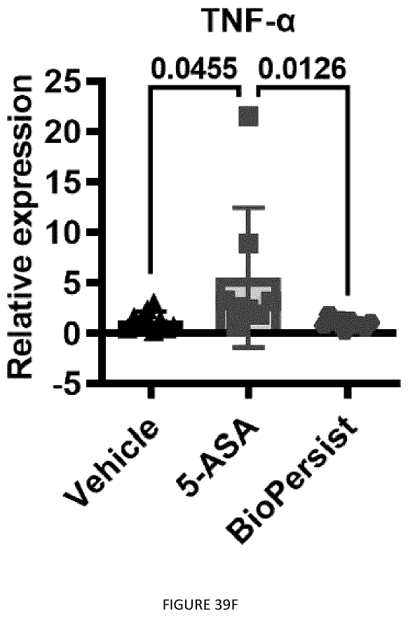

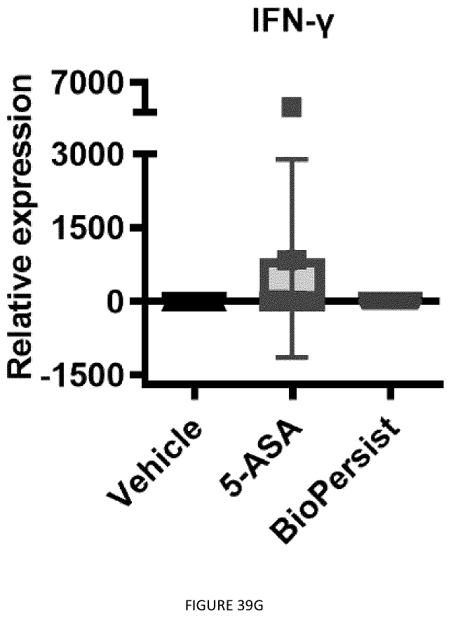

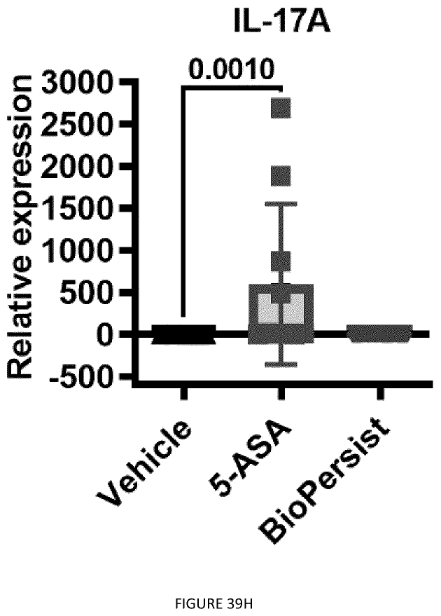

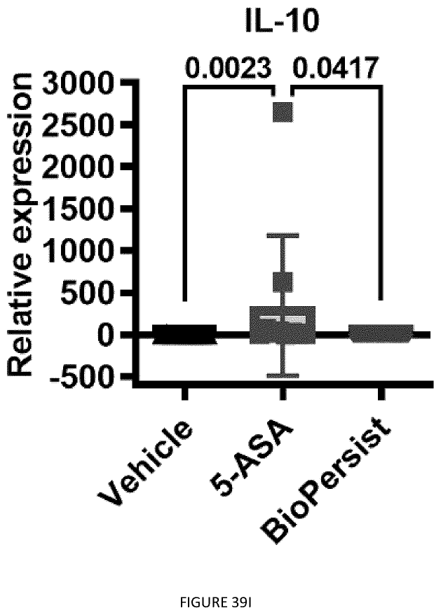

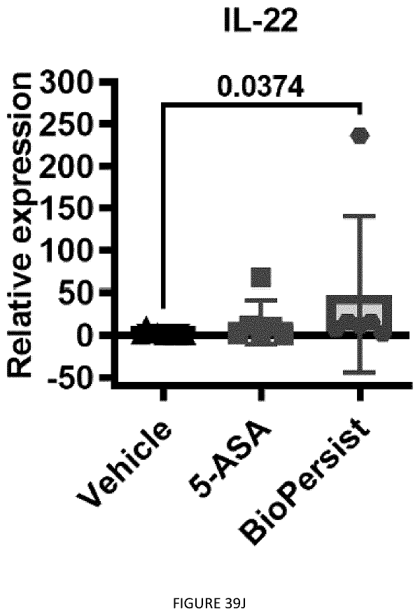

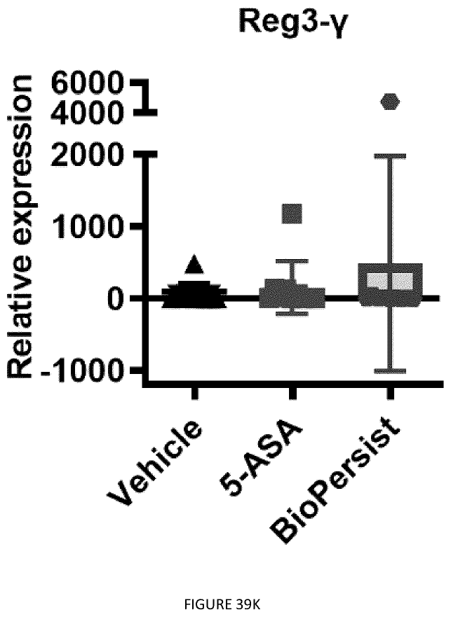

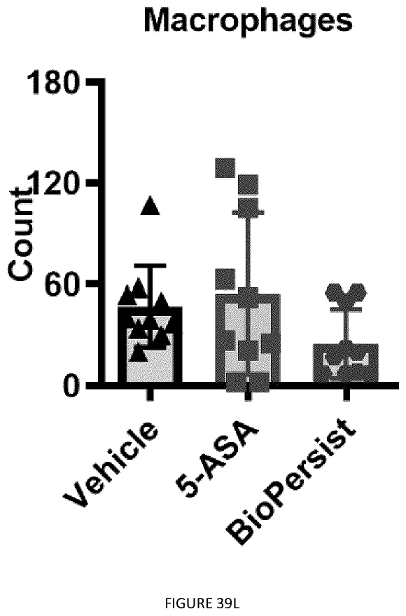

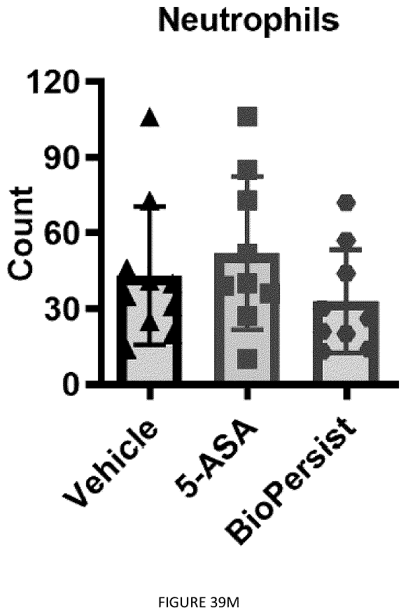

The cornerstone front-line treatment for ulcerative colitis is the anti-inflammatory 5-aminosalicylates (5-ASA). However, 30% of ulcerative colitis patients are refractory to 5-ASA, and within the first year, a further 40% will fail this front-line treatment. Since the designer strain expressing ttr was the most effective designer strain in the DSS-colitis model, we compared its efficacy to 5-ASA, as shown in FIGS. 39A-M. We observed that mice treated with the designer strain had a milder weight loss, and had a lower clinical score, as shown in FIGS. 39A and 39B. When we evaluated the macroscopic changes among the groups, we found that only mice treated with the designer strain had formed stool pellets in the distal colon and had a longer colon length as shown in FIG. 39C. The designer strain expressing ttr was detected in close proximity to the gut epithelium of mice despite 7 days of DSS exposure, as shown in FIG. 39D. The histopathological analysis of the distal colon supported these observations as the designer probiotic intervention group had less epithelial erosion, more crypts with goblets cells and lower immune cell infiltrates, with significantly lower total histopathological damage scores compared to the 5-ASA group, as shown in FIG. 39E. While the designer strain was protective, it appears the mice treated with 5-ASA were refractory given the designer strain was more efficacious, and 5-ASA was similar to the DSS treated mice alone. Indeed, the 5-ASA treated group had higher expression of the pro-inflammatory cytokines TNF-α, IL-17 and IFN-γ as shown in FIGS. 39F-I as well as the highest expression of the anti-inflammatory cytokine IL-10 as shown in FIG. 39I. In contrast, only the designer strain induced IL-22, as shown in FIG. 39J, an important cytokine involved in mucosal healing in inflammatory bowel disease. Both 5-ASA and the designer strain show a similar trend where there is the induction of Reg3-γ, as shown in FIG. 39K, and lower infiltration of macrophages and neutrophils as shown in FIGS. 39K and 39M. Overall, the introduction of the designer strain prior to the challenge to DSS is more protective than the treatment with 5-ASA.

All citations are hereby incorporated by reference.

The present invention has been described with regard to one or more embodiments. However, it will be apparent to persons skilled in the art that a number of variations and modifications can be made without departing from the scope of the invention as defined in the claims.

1. A recombinant probiotic bacterium expressing an N-acetyl-glucosamine binding protein A or fragment or homologue thereof.

2.-4. (canceled)

5. The recombinant probiotic bacterium of claim 1 wherein the N-acetyl-glucosamine binding protein A fragment is an N-terminal fragment.

6. The recombinant probiotic bacterium of claim 5 wherein the N-terminal fragment comprises a mucin binding domain.

7.-10. (canceled)

11. The recombinant probiotic bacterium of claim 1 wherein the N-acetyl-glucosamine binding protein A is from a bacterium from the phyla Gammaproteobacteria, Enterobacteria or Firmicutes.

12.-13. (canceled)

14. The recombinant probiotic bacterium of claim 1 wherein the N-acetyl-glucosamine binding protein A or fragment thereof is co-expressed or recombined with a bacterial surface protein.

15. The recombinant probiotic bacterium of claim 14 wherein the bacterial surface protein A is a mucus binding protein or a fragment thereof.

16. The recombinant probiotic bacterium of claim 15 wherein the N-acetyl-glucosamine binding protein A comprises the mucus binding protein or a fragment thereof.

17.-46. (canceled)

47. The recombinant probiotic bacterium of claim 1 wherein the expression of the N-acetyl-glucosamine binding protein A is chromosomal or plasmid-based.

48. The recombinant probiotic bacterium of claim 1 wherein the recombinant probiotic bacterium is a Lactobacillus, Bifidobacterium, Lactococcus, Enterococcus, Streptococcus, Pediococcus, Leuconostoc, Bacillus, or Escherichia.

49. (canceled)

50. The recombinant probiotic bacterium of claim 1 wherein the recombinant probiotic bacterium is E. coli Nissie 1917 or L. reuteri DSM20016.

51. (canceled)

52. A nucleic acid molecule comprising a nucleic acid sequence encoding an N-acetyl-glucosamine binding protein A or fragment or homologue thereof in combination with a bacterial surface protein.

53.-58. (canceled)

59. A vector comprising the nucleic acid sequence of claim 52.

60. (canceled)

61. A host cell comprising the vector of claim 59.

62. A method of increasing colonization of a probiotic bacterium in the gastrointestinal tract of a subject in need thereof, or of reducing inflammation in the gastrointestinal tract of a subject in need thereof, or of treating or preventing inflammatory bowel disease in a subject in need thereof, the method comprising administering the recombinant probiotic bacterium of claim 1 to the subject.

63.-64. (canceled)

65. The method of claim 62 wherein the subject is a human.

66.-69. (canceled)

70. A probiotic composition comprising the recombinant probiotic bacterium of claim 1 and a pharmaceutically acceptable carrier.

71. A method of ameliorating gastrointestinal inflammation in a human or non-human subject in need thereof comprising administering to the subject an effective amount of the composition of claim 70 sufficient to ameliorate the gastrointestinal inflammation.

72. The method of claim 71, wherein the gastrointestinal inflammation is associated with inflammatory bowel disease.

73. The method of claim 71, wherein the composition is administered orally or rectally.

74. The recombinant probiotic bacterium of claim 1, wherein the recombinant probiotic bacterium is any enteric bacterium.


SEQUENCE LISTING (73 sequences)

SEQ ID NO: 1 - DNA, 21 bp, synthetic construct
  misc_feature 1..21: note = Primer
  source 1..21: mol_type = other DNA
gagctcgaattctcatgtttg

SEQ ID NO: 2 - DNA, 21 bp, synthetic construct
  misc_feature 1..21: note = Primer
  source 1..21: mol_type = other DNA
ggatcctctagagtcgacctg

SEQ ID NO: 3 - DNA, 52 bp, synthetic construct
  misc_feature 1..52: note = Primer
  source 1..52: mol_type = other DNA
gcatgcctgcaggtcgactctagaggatccgttatatacgctcgatttttgc

SEQ ID NO: 4 - DNA, 51 bp, synthetic construct
  misc_feature 1..51: note = Primer
  source 1..51: mol_type = other DNA
ataagctgtcaaacatgagaattcgagctcttattcatggctcatacgttg

SEQ ID NO: 5 - DNA, 20 bp, synthetic construct
  misc_feature 1..20: note = Primer
  source 1..20: mol_type = other DNA
cgttatggactgcaacatgg

SEQ ID NO: 6 - DNA, 20 bp, synthetic construct
  misc_feature 1..20: note = Primer
  source 1..20: mol_type = other DNA
gcaaacggcctaaatacagc

SEQ ID NO: 7 - DNA, 60 bp, synthetic construct
  misc_feature 1..60: note = Primer
  source 1..60: mol_type = other DNA
tgccaagcttgcatgcctgcaggtcgactctagaggatccattccggggatccgtcgacc

SEQ ID NO: 8 - DNA, 60 bp, synthetic construct
  misc_feature 1..60: note = Primer
  source 1..60: mol_type = other DNA
ctgatcagtgataagctgtcaaacatgagaattcgagctctgtaggctggagctgcttcg

SEQ ID NO: 9 - DNA, 60 bp, synthetic construct
  misc_feature 1..60: note = Primer
  source 1..60: mol_type = other DNA
ccaattactaccagcttcagcactaccagcactaccaatcctctttcggtaataaatctt

SEQ ID NO: 10 - DNA, 60 bp, synthetic construct
  misc_feature 1..60: note = Primer
  source 1..60: mol_type = other DNA
gtgagcgcgcgtaatacgactcactatagggcggatccggtctatcctttatgggagaac

SEQ ID NO: 11 - DNA, 90 bp, synthetic construct
  misc_feature 1..90: note = Primer
  source 1..90: mol_type = other DNA
gtgctgaagctggtagtaattggagtcatccacaatttgaaaaaggtagtgctggtagtgctgctggtagtcacggttacgtatcggcag

SEQ ID NO: 12 - DNA, 60 bp, synthetic construct
  misc_feature 1..60: note = Primer
  source 1..60: mol_type = other DNA
aattcaccactaccagcagcactaccagcactaccaccgtcaaacttaacgtcaataacg

SEQ ID NO: 13 - DNA, 60 bp, synthetic construct
  misc_feature 1..60: note = Primer
  source 1..60: mol_type = other DNA
agtgctggtagtgctgctggtagtggtgaatttaaagttacctatagtggtagtgacagc

SEQ ID NO: 14 - DNA, 60 bp, synthetic construct
  misc_feature 1..60: note = Primer
  source 1..60: mol_type = other DNA
cgatatcaagcttatcgataccgtcgacctcgagaattcccgtcaagataatccgataag

SEQ ID NO: 15 - DNA, 28 bp, synthetic construct
  misc_feature 1..28: note = Primer
  source 1..28: mol_type = other DNA
gaattgggtaccgggccccccctcgagg

SEQ ID NO: 16 - DNA, 28 bp, synthetic construct
  misc_feature 1..28: note = Primer
  source 1..28: mol_type = other DNA
gccctatagtgagtcgtattacgcgcgc

SEQ ID NO: 17 - DNA, 22 bp, synthetic construct
  misc_feature 1..22: note = Primer
  source 1..22: mol_type = other DNA
aactgttggggttacttcggta

SEQ ID NO: 18 - DNA, 21 bp, synthetic construct
  misc_feature 1..21: note = Primer
  source 1..21: mol_type = other DNA
ctggttgttgctcaggtgttt

SEQ ID NO: 19 - protein, 485 aa, Vibrio cholerae
MKKQPKMTAIALILSGISGLAYGHGYVSAVENGVAEGRVTLCKFAANGTGEKNTHCGAIQYEPQSVEGPDGFPVTGPRDGKIASAESALAAALDEQTADRWVKRPIQAGPQTFEWTFTANHVTKDWKYYITKPNWNPNQPLSRDAFDLNPFCVVEGNMVQPPKRVSHECIVPEREGYQVILAVWDVGDTAASFYNVIDVKFDGNGPVLPDWNPAGQIIPSMDLSIGDTVYTRVFDNDGENPAYRTELKIDSETLTKANQWSYALATKINQTQKQQRAGQLNGDQFVPVYGTNPIYLKEGSGLKSVEIGYQIEAPQPEYSLTVSGLAKEYEIGEQPIQLDLTLEAQGEMSAELTVYNHHQKPLASWSQAMTDGELKSITLELSEAKAGHHMLVSRIKDRDGNLQDQQTLDFMLVEPQTPPTPGDYDFVFPNGLKEYVAGTKVLASDGAIYQCKPWPYSGYCQQWTSNATQYQPGTGSHWEMAWDKR

SEQ ID NO: 20 - protein, 180 aa, Vibrio cholerae
HGYVSAVENGVAEGRVTLCKFAANGTGEKNTHCGAIQYEPQSVEGPDGFPVTGPRDGKIASAESALAAALDEQTADRWVKRPIQAGPQTFEWTFTANHVTKDWKYYITKPNWNPNQPLSRDAFDLNPFCVVEGNMVQPPKRVSHECIVPEREGYQVILAVWDVGDTAASFYNVIDVKFDG

SEQ ID NO: 21 - protein, 1229 aa, Lactobacillus reuteri
MYLGGLIMLSRKNYKETIRKQTPTKQYYTIKKLTVGVTSVLIGLSFMGELEGDSVHADTMTASSESTSVTSTTAQDGLKKSPQLYLQVTDTNNPSTPLSASSTGTSKNVTSSAAVQVKSASDEEDSDSTLAKGENKFARSAVKDSVTDGKTSTAEINPAKLSSPALITQLNQSLAKSSTSDAAKANDELEIKATDPTNYPNCGDVYGPLFELDASGQLVNKDEVISLKDMYIFQILKLVNTKDSDFQYVILTMNRKDTADRSVYLFVTGSNYSNAVVVKVKPNDTYELSKTGYSVTYTEPTTINGHYVDGTFYVTGSTYDDGFIMPDWQLQHLQIIYSLGNYDPSNTDATSVCEIMPSYEKVPVIKYSGVPSNISQPKVYITGFTGQEFNVTDIINNYKKVFKGYYLQNPNVASMGTLSQFENGGYYLKTYYDNDGNVDFKGLYHQIDDQGTMSVSVLNADNKTIVGPENILAGKSHNFNFNGHNWIARNPYVTSSAHEVILKYAKLGSVIPVDENGNKINDGWQYVNDPDDASKATSPYEKAPVIDGYVAVNPDETIVLPHNLSSDTKIYYRKRIKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIVTVNYKGYDKKVYDGQPGTINPGKLTWSKLPDGTSLKMPTWSIDDFAWETADGLAPTAVGTYRIILTDAGKAALKKINPNYDLSSITGVFTYEIKPAQTPEILGQTPEQQPGQNTNQSGAENGFGSSTRPNASTNSNLNQLPQTGNEHSNTALAGLALAFLTAMLGLGKKRKHD

SEQ ID NO: 22 - DNA, 540 bp, synthetic construct
  misc_feature 1..540: note = Harmonized Sequence
  source 1..540: mol_type = other DNA
cacggttacgtatcggcagttgaaaacggtgtagccgaagggcgtgtaactctttgtaaatttgcagccaacggtacaggggagaaaaacacacactgtggtgcaattcaatatgaacctcaatctgtagaaggtcctgatggtttccctgtaacaggtcctcgtgatggtaaaattgcctctgcagaatctgcccttgcagccgcacttgatgaacaaactgcagaccgttgggtcaagcgtcctattcaagcaggtcctcaaactttcgagtggaccttcactgcaaaccacgtaacgaaggattggaagtactacattactaagccaaactggaacccaaaccagcctcttagccgtgatgcatttgacttgaaccctttctgtgtcgtagaagggaacatggttcagcctcctaagcgtgtatctcacgaatgtattgttcctgaacgtgaagggtaccaggtaatcctagcagtctgggatgtaggtgatactgcagcctcgttctacaacgttattgacgttaagtttgacggt

SEQ ID NO: 23 - DNA, 3690 bp, Lactobacillus reuteri
  source 1..3690: mol_type = genomic DNA
ttgtatttaggagggttaataatgctatcaagaaaaaattataaggaaactatacgaaaacagacacctacaaaacagtactatactattaagaaattaactgttggggttacttcggtattaattggtctatcctttatgggagaactagaaggggatagcgttcatgcggacacgatgacagcaagcagtgagtcaacaagtgttacgtcgacgactgctcaggatggtttaaaaaaatctccacaactctatttgcaagttactgatacaaataacccaagtacaccattaagtgcttcatccacagggactagtaagaatgttacctcatcagctgcggtacaagtgaagtccgctagtgatgaagaagatagtgattctacactagctaagggagaaaataaatttgctcggtcagcagtaaaagattcagtcactgatgggaaaacaagtacagcagaaattaatccggcaaaattaagcagtcctgctttaataacgcaactcaaccaatccttagctaagagcagtacgagtgatgcagcaaaagctaatgatgagttagaaattaaagcaacagatccgactaattatccaaactgtggcgatgtgtatgggccattatttgaattggatgctagcggacagcttgttaataaagatgaagttatatctcttaaagatatgtatattttccaaatattgaaattagtaaatacaaaagatagtgactttcaatatgtaatattaacaatgaatcgtaaagatactgcagataggtctgtatatctttttgtaactggaagcaattatagtaatgctgttgttgttaaagtaaagccaaatgatacttatgaattaagtaaaactggatatagtgttacttatacagaaccaacaactataaatggacattatgttgatggaactttttatgttacaggaagtacttacgatgatggttttataatgccagattggcaactgcagcaccttcagattatatatagtttaggaaattatgatccaagcaatactgacgcaacatcagtttgtgaaataatgccaagttatgaaaaggtaccggtaattaaatatagtggagtaccttcaaatattagccaacctaaggtttacattaccgggtttacgggtcaagagtttaacgttacagatattattaacaattataagaaagtttttaagggctactatcttcaaaatcctaatgtggcgtccatgggaactctttcccaatttgagaatggtggttattacttaaagacatattatgataatgatggtaatgttgactttaagggcttgtatcatcaaattgatgatcagggaacaatgagtgtgagtgttcttaatgcagataataaaacaattgttggacctgaaaatattcttgctggtaaatcgcataactttaactttaatggtcataactggattgcgcggaatccttatgtcactagttcagctcacgaagtcatattaaagtatgctaagttaggttcagttattcctgttgatgaaaacggaaataaaataaacgatggatggcaatatgttaatgatccagatgatgcttccaaagccactagcccatatgaaaaagcgccagttatcgatggttatgtagctgtaaatccagatgaaacgatcgttcttcctcataacttaagtagtgacacaaagatttattaccgaaagaggattaaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgtgactgtaaattacaagggctatgataagaaagtctatgatggtcaacctggaacgattaatccgggtaaattaacgtggagtaagttgccagatggtacttcattgaagatgccaacatggagtatagatgatttcgcttgggaaacagctgatggcttagcaccaacggcagtaggaacttatcggattatcttgacggatgctggtaaggctgcactaaagaagattaatccaaattatgacttaagcagtattactggtgtctttacttatgaaattaagccagcacagacaccagaaatcttaggccaaacacctgagcaacaaccaggccaaaatactaatcaatcaggagctgaaaacggctttggttcttctacaaggcctaatgcatcaactaactccaatcttaatcaacttccacagactggtaatgagcattctaatactgcacttgctggtctagcattggctttcttgactgctatgcttggtttgggcaagaagcgtaaacatgattag

SEQ ID NO: 24 - DNA, 4350 bp, synthetic construct
  misc_feature 1..4350: note = GbpA-MBP Chimeric Sequence
  source 1..4350: mol_type = other DNA
ttgtatttaggagggttaataatgctatcaagaaaaaattataaggaaactatacgaaaacagacacctacaaaacagtactatactattaagaaattaactgttggggttacttcggtattaattggtctatcctttatgggagaactagaaggggatagcgttcatgcggacacgatgacagcaagcagtgagtcaacaagtgttacgtcgacgactgctcaggatggtttaaaaaaatctccacaactctatttgcaagttactgatacaaataacccaagtacaccattaagtgcttcatccacagggactagtaagaatgttacctcatcagctgcggtacaagtgaagtccgctagtgatgaagaagatagtgattctacactagctaagggagaaaataaatttgctcggtcagcagtaaaagattcagtcactgatgggaaaacaagtacagcagaaattaatccggcaaaattaagcagtcctgctttaataacgcaactcaaccaatccttagctaagagcagtacgagtgatgcagcaaaagctaatgatgagttagaaattaaagcaacagatccgactaattatccaaactgtggcgatgtgtatgggccattatttgaattggatgctagcggacagcttgttaataaagatgaagttatatctcttaaagatatgtatattttccaaatattgaaattagtaaatacaaaagatagtgactttcaatatgtaatattaacaatgaatcgtaaagatactgcagataggtctgtatatctttttgtaactggaagcaattatagtaatgctgttgttgttaaagtaaagccaaatgatacttatgaattaagtaaaactggatatagtgttacttatacagaaccaacaactataaatggacattatgttgatggaactttttatgttacaggaagtacttacgatgatggttttataatgccagattggcaactgcagcaccttcagattatatatagtttaggaaattatgatccaagcaatactgacgcaacatcagtttgtgaaataatgccaagttatgaaaaggtaccggtaattaaatatagtggagtaccttcaaatattagccaacctaaggtttacattaccgggtttacgggtcaagagtttaacgttacagatattattaacaattataagaaagtttttaagggctactatcttcaaaatcctaatgtggcgtccatgggaactctttcccaatttgagaatggtggttattacttaaagacatattatgataatgatggtaatgttgactttaagggcttgtatcatcaaattgatgatcagggaacaatgagtgtgagtgttcttaatgcagataataaaacaattgttggacctgaaaatattcttgctggtaaatcgcataactttaactttaatggtcataactggattgcgcggaatccttatgtcactagttcagctcacgaagtcatattaaagtatgctaagttaggttcagttattcctgttgatgaaaacggaaataaaataaacgatggatggcaatatgttaatgatccagatgatgcttccaaagccactagcccatatgaaaaagcgccagttatcgatggttatgtagctgtaaatccagatgaaacgatcgttcttcctcataacttaagtagtgacacaaagatttattaccgaaagaggattggtagtgctggtagtgctgaagctggtagtaattggagtcatccacaatttgaaaaaggtagtgctggtagtgctgctggtagtcacggttacgtatcggcagttgaaaacggtgtagccgaagggcgtgtaactctttgtaaatttgcagccaacggtacaggggagaaaaacacacactgtggtgcaattcaatatgaacctcaatctgtagaaggtcctgatggtttccctgtaacaggtcctcgtgatggtaaaattgcctctgcagaatctgcccttgcagccgcacttgatgaacaaactgcagaccgttgggtcaagcgtcctattcaagcaggtcctcaaactttcgagtggaccttcactgcaaaccacgtaacgaaggattggaagtactacattactaagccaaactggaacccaaaccagcctcttagccgtgatgcatttgacttgaaccctttctgtgtcgtagaagggaacatggttcagcctcctaagcgtgtatctcacgaatgtattgttcctgaacgtgaagggtaccaggtaatcctagcagtctgggatgtaggtgatactgcagcctcgttctacaacgttattgacgttaagtttgacggtggtagtgctggtagtgctgctggtagtggtgaatttaaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgtgactgtaaattacaagggctatgataagaaagtctatgatggtcaacctggaacgattaatccgggtaaattaacgtggagtaagttgccagatggtacttcattgaagatgccaacatggagtatagatgatttcgcttgggaaacagctgatggcttagcaccaacggcagtaggaacttatcggattatcttgacggatgctggtaaggctgcactaaagaagattaatccaaattatgacttaagcagtattactggtgtctttacttatgaaattaagccagcacagacaccagaaatcttaggccaaacacctgagcaacaaccaggccaaaatactaatcaatcaggagctgaaaacggctttggttcttctacaaggcctaatgcatcaactaactccaatcttaatcaacttccacagactggtaatgagcattctaatactgcacttgctggtctagcattggctttcttgactgctatgcttggtttgggcaagaagcgtaaacatgattag

SEQ ID NO: 25 - DNA, 7373 bp, Salmonella enterica
  source 1..7373: mol_type = genomic DNA
ttattcatggctcatacgttgttcgtattctggtctctggcgaggccattttttcgaaacgcctaatcagttccgccaggctaccggcctgcattttttccatgactctggcgcggtgcacctctacggtacgcaccgcgatattcatcgcttccgcaatttcacggttcataaatccttttgccaccaggctggccagctcacgctctttcggcgtcaactgctggtaacacagtataatctcacgacgcgccaccgctgccgatgaaaccgtcagcgcacgctccagcgccgcctgtagcggttttaccgataccggtttttgcagaaaatcgacggcgccgcgtttcatctgctccacggccatcggtacatcgccatgcccggtaagaaaaacaaccgccagggtacttccgcactggcgcaacgcatcatgaacgccctgcccatccagtaccggcattcgcatatccagtaatacgaccccggcctgatacagactggcctgcgccaaaaaatccgccccctgcgtccagcattttacgtcatatcccagactttccagtaaaaacgcgcacgcgttagtgaccgccgtatcatcatccagtagatgaattgtcgccatccctgcccccattttcatgtaagaaatgtatcgtaaccaccgttcccgacagaccgtccggcgcggtctggttcctgatgctgatatcgccccgcccataccgcaccagccgctggcaaatcgccagccctaagcccatcccctctttacgggtggtcataaacggctgaaacgcctgacgtaatagcgcctcatcgattcccccggcgttatcctgtaaaacaatactgatgccgttttcagtgcgttcagcaacgatccataaatgggtggcgcccgcctgagccgcattaagaatgatattcgccagcacctgttccagcagcactgacggcagcgttacgcgcagcgcagcgctaacctcggtatgcagagtcactgtcggaaactgttgcgccatacgcaacaattgccagacatgatcaatcgcctcgcgaatggctatggccttccacgcttcggttagcaccgggttgccctgcgcctggctgacccagtgacgcaggttacgcagagtatccgcaccgcgttgcgcctgctggtcaatctgctccagcgccggcagcaagggatgctgttcatctgcagcgcgcagtcgaatcaggcacccctgggcataatgtcgaatcgcggaaagcggctgattaagctcatgggcaaacccggaggtcatttcacccaacacgctcatttgccgggcggtttccagcgcccgctcatgctgatgaagaactacgctattacgttccagttgctttccacgtcgacgcaccagcagcatgacccaaatataattgagcgtgagcaacaagaacgccagaatcacgccgccgaccattagctggtgctggattaaccaacttttgacatccagccacagtcgacgctgctgagggtgctgacgaacatcacgcagcaaggcttccacctgactggtggacgcaggcgcgccccagtgaaatgacgcggcggcgggcgcgttgaatagcgctcgcgttacgcgatccgccagcgcatcgcttaccgcaggtagcgccgcgaacgaccagtcaggatataacggcgtactggttaagcaaggcaggggcgtcggtcgggaaagcagcgcgataaagtcctttttattaatcaatccttcctgatccatattttctaacaggcacactggcacaattgccgcctgcaccgctttttcgcgcagcatatagactaaggcatcgccaggaaatccggtaaaacggagatgaaaatcgcgctccgggcgtaagcccgcgtcgctgagcgctttatagcctaataaatagccgccaaacgcctgagcatcaatcgcgccgacggtcttaccgatgagatcatgcgccgtggtgatgccgctatcgcgccgggtcaaaatcacgctgccaataacattactcaccgctttcccatcgcgcgtggagcgcagggaagctaaccagcgcagcggcgcatggctgttcagttggacaaattgcgccgggttggttatcacaaactgcacggttccctggttaacggcctcctgcatttgatgcagatccagcggctggatgtgaaaggtttcgcctggaagctgttggcttaatgtctttgccaacggttgccagtggctacgcgtagacgcctcgccgcgcatggccaaaataccgatattccacgtccctgcccacgcgccatgacaaagtagccctactgccgccaacaccgccaggcgccttacggttttacctctcaccccaatatccctgtcaattatgttgttttagatcaacaacaagccgggtatgtggttaaccacaatagagcgcaccccgcctcgatttttacactgtaaatcatcgacattttttattcattacacatgaaccaacatcgtgacaaatgtttcattgttggcaatgtggacgggagtcaatatggacagcagtaaacggcaatttctccagcagcttggcgtcctgaccgctggcgcctcgctggttccgctggctgaagcgaaatttcctttttcgccggagcggcatgaaggctctccccgacaccgttacgccatgcttatcgatctgcggcgttgtatcggctgtcagtcctgtaccgtaagttgcactattgaaaaccaaacgccgcaaggcgcgtttcgtacgacggtgaaccaataccaggtccagcgtgaaggtagtcaggaagtcacgaatgtgctgttgccgcgtctgtgcaaccattgcgataaccccccctgtgtgccggtctgcccggtacaagccacctttcagcgggaagatggcattgtggtggtggataacaaacgctgcgtcggctgcgcctattgtgtccaggcgtgtccttacgacgcccgatttatcaatcatgaaacgcaaactgccgataaatgcacgttttgcgtccatcgtctggaagccggactgttacccgcttgcgtagagtcctgcgtcggcggcgcgcgtattattggcgatatcaaagatccccatagccgcatcgccaccatgcttcatcagcatcgcgacgctatcaaggtattaaagccggaaaacggcacgtcgccccatgttttctacctgggtctggacgacgcctttgtcaccccattaatgggccgtgcgcagcccgcgctttggcaggaggtctgaatgacgcattcactcatcattgaagaagtgctggctcacccgcaggacattagctggctgccgtgggcggtacaatatttcttttttattggcattgccgcctgcgccgcactgtttgcctgttatcttcactggcggaaaaaagacgccgcaacagaagaaaatcgggcattactgattgccattacctgtgcgattaccgcaccgctggcgctgacggcggatctgcaccagaccgcccgcgtctggcatttctatgcctggccgacgccctggtcgtggatgccctggggagcgttattcctgccgctgtttaccggatttctcgctctgtggttcctggcgcagcagattaaacgattattcaataaaagttacaacgtcactaaatggttggcgttagccagcgcgctttgcgcggtgggcctgttgatttataccggccgcgaagtctccgttgtgctggcgcgcccaatctggtttagctacgccttccccgtggcgatgtttcttagcgccttacaggcgttcttcgcgctgatgattgtcgccgcccgacgcgactcggtaaggctgccaaaaatattgtggggacaaatctggacgctggcggcgctggggctggttgtggccatgtgggttagcggcgatacgctttccggcacggcaatccgtcagtggattaccgtcgccctgtcagccaaatattacgctgtcggctgggtagcgctgtgggtatgcacactgctgttctgtagcctggcgctacgccatccgttatcacagctaagacgcgtcctgctggttctcagcgcgctggcgctatgttggctgatgcgctggacattgttgattcaggtacaaaccgtccccaagttcaacgcgcaatttaacccttactcgttaccaggcggaacggatggctggctggctattctcggcaccttcggcctgtggatagcgctactgattattattcgtgaaacgctgaacggactcaccaggagattacaacatggctaatttaacccgtcgtcagtggctaaaagtcggtctcgccgtcggtgggatggtcacttttggtctgagctaccgtgatgtggcgaaacgcgcaattgatggcctgttaaacgggacgtccggcaaggtaacgcgcgaccgcatctttggcaatgcgttaattccggaggcgcaggcgcaaacacactggcagcaaaatccacaacaaaccatcgccatgacgcaatgcttcggctgttggacacagtgcggtatccgcgcccgggttaatgccgatggcaaagtgatacgcatcgccggcaatccctatcaccccttgtcgcaggaacacccgattgactcgtccgtcccttttagcgaagccatggagcaactggcgggagaaagcggtcttgacgcccgctcaaccgcctgcgcgcgcggcgccacgctgctggaaagcctgtacagtccgctacgactgcttgaaccgatgaaacgcgtgggtaaacgcggcgaagggaaatggcagcgcatcagctttgagcaacttattgaagaagtcgtggaaggcggcgatctgtttggcgaaggtcatgtggacggactgcgcgctattcatgcgccggatacgccaattgacgcaaagcaccccagtttcgggcccaaaaccaatcagttactggtcacgaataccagcgacgaaggccgcgatgcgtttctgcgtcgttttgcgctaaatagcttcggcagcaagaatttcggcgcgcatggcgcctactgtggactggcttaccgggccggctccggggcattgatgggcgatctggataaaaacccgcatgtcaaacccgactgggaaaacgtggagtttgcgctctttatgggcacctccccggcacagtccggcaatccgtttaaacgccaggcacgtcagttggcgagcgcccgactgcgtgagaattttcaatacgtcgtggtcgcccccgccctccccttatcaacggtgctcgccgatcctcgcggtcgctggcaaccggtcatgcccggcagtgattcggcgctggcaatggggatgatccgctggatcatggataatcaacgttataatgctgattatctggcgattcccggcgtacaggcgatgcagcaggccggcgagcaaagttggaccaacgccacgcacctggtcattgcggatgagctgccgacgcttgccggacaacacctgacgctgcgccatcttacgcccgatggcgaagagacccctgtcgtactgaataccgacggcgagttggtcgatgcgtccacttgccgacaggcacggcttttcgtgacgcagtacgttacgctcgccgacggccaacgggtcacggtgaagagcgggttgcaacgcctgaaagaggcggcagaaaagctctcgttggcgcaatacagcgaacagtgcggcgtgccggaagcgcaaattatcgcgctggcggaaacctttaccagtcacggacgtaaagctgcggtcatcagtcacggcggcatgatggccggcaatgggttttataacgcctggtcggtcatgatgcttaacgcgctgatcggcaacctcagcttgtccggcggcgtctttgtcggcggcggcaaattcaacggcgttagcgacggcccccgctacaacatgaacagttttgccggaaaagtgaaaccgtccgggttaagtattgcccgtagcaaaaccgcttatgaagcatcggaagaataccgcgacaaaattgccggtgggcaatccccttatccagccaaagcgccgtggtatccctttgtggcaggccagcttaccgaactgttgacctccgcgctcgaaggctatccttatccgcttaaagcctggatttccaatatgagcaacccgttttacggtgttcccggtctacgcgccgtggcggaagaaaaactaaaagaccctcgccgactgccgctctttatcgcgattgacgcctttatgaatgaaacgacggcgctggcggattacattgtgccggatacgcacaattttgagagctggggctttacggcgccctggggcggcgtagccagtaaagccactaccgcccgctggccggttgtcgcccccgccactcaccgcacggcggacgggcaacctgtctcaatggaagcattttgtattgcggtagcaaaacggctccatctgcccggcttcggcgaccgggcgataaccgatccgcagggcaatacttttccactgaaccgggcggaagacttctatctgcgcgtagccgctaatatcgcctttatgggcaagacgccggtcgcgctggcaaatcaggaagatatttcgcttaccggcgtcagccgcattctgccagcaattcagcacacgcttaaagctgatgaggtcggtcgcgtggcgtttatctactcgcgtggcggccggtttgcgcccgaggatagcggctatacggagcaacggttaggtaacgcgtggaaaaaacccttacagatctggaatgcagatgtcgccgcccaccgtcacgccatcaccggggagcgcttcagcggttgcccggtctggtatccggcgcgtttgtcagatggtcgtgcgattgacgaccagtttcccattgggcaatggccgctgaaactgatttcatttaaatcaaataccatgtccagctcaacagccgtcatcccgcgcttacaccatgtgaagccagcaaacctggtggcgctgaatccgcaagacggcgagcgttatggactgcaacatggcgatcgggtacggatcattacgccgggcggtcaggtcgtggcgcaaatcagtttgttaaatggcgtgatgccaggcgtcatcgccatcgaacacggatatggccaccgcgagatgggcgcaacgcagcactctctggatggcgtgcctatgccgtatgatccacaaatcagggcaggcataaatcttaacgatctgggctttgccgatccgacaagaaccattaccaacacctggctcgactgggtttctggcgcggcagtacgtcaggggctgccggcaaaaatcgagcgtatataac

SEQ ID NO: 26 - DNA, 1458 bp, Vibrio cholerae
  source 1..1458: mol_type = genomic DNA
atgaaaaaacaacctaaaatgaccgctattgccctgatcctctctggtatcagtggattagcgtatggacacggctacgtttccgcagtggaaaacggtgtcgccgaaggacgtgtcaccttgtgtaaatttgccgctaacggcactggagagaaaaacactcactgtggcgcgattcaatacgaaccacaaagtgtcgaaggcccagatggcttcccggtcactggccctcgtgatggcaaaattgccagtgcggaatcggcactggcggcagcgctggatgagcaaaccgccgaccgttgggtaaagcgcccaattcaagctggcccacaaaccttcgagtggacgttcaccgccaaccacgtcacaaaggattggaaatactacattaccaaaccaaactggaacccaaaccagccattgtcgcgtgatgcatttgacctcaatccgttctgtgtcgttgaaggaaatatggtgcagccaccaaaacgtgtcagccacgaatgtatcgtgcctgagcgcgaagggtatcaggtcatcctcgccgtatgggatgttggcgataccgcagcttccttctacaacgtgatcgacgtgaaatttgacggtaacggcccagtgttacccgattggaacccagcaggtcaaatcattccaagtatggatctcagcattggcgataccgtgtacactcgcgtgtttgataacgatggggaaaaccctgcttatcgcactgagctaaaaattgactctgagacgctaaccaaagccaatcaatggtcttacgctctggcgactaaaattaaccaaacgcaaaaacagcaacgtgctggtcagcttaatggcgatcaatttgttcccgtttacggcaccaacccgatttatctgaaagaaggcagtggcttgaagagtgttgaaattggctaccaaattgaagcgccacagcctgagtattcactgacggtttctggtctagcgaaagagtatgagattggcgaacaaccgattcagcttgacctgactttagaagcgcaaggtgaaatgagcgcagagctgaccgtgtataaccaccaccaaaaaccgctggcaagttggtcacaagcgatgacggatggcgagctgaaatccatcacgctagagctgagcgaagctaaagcgggacatcatatgttggtttctcgcatcaaagatcgcgatggcaatctgcaagatcaacaaactctcgatttcatgctggttgaaccgcaaacaccaccaacaccgggtgactacgactttgtgttcccgaatggcctgaaagagtacgtggctggcaccaaagtgctcgctagtgatggcgcaatctaccaatgtaagccatggccatactctggctactgccagcaatggacaagtaacgctactcaataccaaccgggtactggcagtcattgggaaatggcgtgggataaacgttaa

SEQ ID NO: 27 - DNA, 540 bp, Vibrio cholerae
  source 1..540: mol_type = genomic DNA
atgaaaaaacaacctaaaatgaccgctattgccctgatcctctctggtatcagtggattagcgtatggacacggctacgtttccgcagtggaaaacggtgtcgccgaaggacgtgtcaccttgtgtaaatttgccgctaacggcactggagagaaaaacactcactgtggcgcgattcaatacgaaccacaaagtgtcgaaggcccagatggcttcccggtcactggccctcgtgatggcaaaattgccagtgcggaatcggcactggcggcagcgctggatgagcaaaccgccgaccgttgggtaaagcgcccaattcaagctggcccacaaaccttcgagtggacgttcaccgccaaccacgtcacaaaggattggaaatactacattaccaaaccaaactggaacccaaaccagccattgtcgcgtgatgcatttgacctcaatccgttctgtgtcgttgaaggaaatatggtgcagccaccaaaacgtgtcagccacgaatgtatcgtgcctgagcgcgaagggtatcaggtcatc

SEQ ID NO: 28 - protein, 1222 aa, Lactobacillus reuteri
MLSRKNYKETIRKQTPTKQYYTIKKLTVGVTSVLIGLSFMGELEGDSVHADTMTASSESTSVTSTTAQDGLKKSPQLYLQVTDTNNPSTPLSASSTGTSKNVTSSAAVQVKSASDEEDSDSTLAKGENKFARSAVKDSVTDGKTSTAEINPAKLSSPALITQLNQSLAKSSTSDAAKANDELEIKATDPTNYPNCGDVYGPLFELDASGQLVNKDEVISLKDMYIFQILKLVNTKDSDFQYVILTMNRKDTADRSVYLFVTGSNYSNAVVVKVKPNDTYELSKTGYSVTYTEPTTINGHYVDGTFYVTGSTYDDGFIMPDWQLQHLQIIYSLGNYDPSNTDATSVCEIMPSYEKVPVIKYSGVPSNISQPKVYITGFTGQEFNVTDIINNYKKVFKGYYLQNPNVASMGTLSQFENGGYYLKTYYDNDGNVDFKGLYHQIDDQGTMSVSVLNADNKTIVGPENILAGKSHNFNFNGHNWIARNPYVTSSAHEVILKYAKLGSVIPVDENGNKINDGWQYVNDPDDASKATSPYEKAPVIDGYVAVNPDETIVLPHNLSSDTKIYYRKRIKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIVTVNYKGYDKKVYDGQPGTINPGKLTWSKLPDGTSLKMPTWSIDDFAWETADGLAPTAVGTYRIILTDAGKAALKKINPNYDLSSITGVFTYEIKPAQTPEILGQTPEQQPGQNTNQSGAENGFGSSTRPNASTNSNLNQLPQTGNEHSNTALAGLALAFLTAMLGLGKKRKHD

SEQ ID NO: 29 - protein, 1442 aa, synthetic construct
  REGION 1..1442: note = GbpA-MBP Chimeric Sequence
MLSRKNYKETIRKQTPTKQYYTIKKLTVGVTSVLIGLSFMGELEGDSVHADTMTASSESTSVTSTTAQDGLKKSPQLYLQVTDTNNPSTPLSASSTGTSKNVTSSAAVQVKSASDEEDSDSTLAKGENKFARSAVKDSVTDGKTSTAEINPAKLSSPALITQLNQSLAKSSTSDAAKANDELEIKATDPTNYPNCGDVYGPLFELDASGQLVNKDEVISLKDMYIFQILKLVNTKDSDFQYVILTMNRKDTADRSVYLFVTGSNYSNAVVVKVKPNDTYELSKTGYSVTYTEPTTINGHYVDGTFYVTGSTYDDGFIMPDWQLQHLQIIYSLGNYDPSNTDATSVCEIMPSYEKVPVIKYSGVPSNISQPKVYITGFTGQEFNVTDIINNYKKVFKGYYLQNPNVASMGTLSQFENGGYYLKTYYDNDGNVDFKGLYHQIDDQGTMSVSVLNADNKTIVGPENILAGKSHNFNFNGHNWIARNPYVTSSAHEVILKYAKLGSVIPVDENGNKINDGWQYVNDPDDASKATSPYEKAPVIDGYVAVNPDETIVLPHNLSSDTKIYYRKRIGSAGSAEAGSNWSHPQFEKGSAGSAAGSHGYVSAVENGVAEGRVTLCKFAANGTGEKNTHCGAIQYEPQSVEGPDGFPVTGPRDGKIASAESALAAALDEQTADRWVKRPIQAGPQTFEWTFTANHVTKDWKYYITKPNWNPNQPLSRDAFDLNPFCVVEGNMVQPPKRVSHECIVPEREGYQVILAVWDVGDTAASFYNVIDVKFDGGSAGSAAGSGEFKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIVTVNYKGYDKKVYDGQPGTINPGKLTWSKLPDGTSLKMPTWSIDDFAWETADGLAPTAVGTYRIILTDAGKAALKKINPNYDLSSITGVFTYEIKPAQTPEILGQTPEQQPGQNTNQSGAENGFGSSTRPNASTNSNLNQLPQTGNEHSNTALAGLALAFLTAMLGLGKKRKHD

SEQ ID NO: 30 - DNA, 4329 bp, synthetic construct
  misc_feature 1..4329: note = GbpA-MBP Chimeric Sequence
  source 1..4329: mol_type = other DNA
atgctatcaagaaaaaattataaggaaactatacgaaaacagacacctacaaaacagtactatactattaagaaattaactgttggggttacttcggtattaattggtctatcctttatgggagaactagaaggggatagcgttcatgcggacacgatgacagcaagcagtgagtcaacaagtgttacgtcgacgactgctcaggatggtttaaaaaaatctccacaactctatttgcaagttactgatacaaataacccaagtacaccattaagtgcttcatccacagggactagtaagaatgttacctcatcagctgcggtacaagtgaagtccgctagtgatgaagaagatagtgattctacactagctaagggagaaaataaatttgctcggtcagcagtaaaagattcagtcactgatgggaaaacaagtacagcagaaattaatccggcaaaattaagcagtcctgctttaataacgcaactcaaccaatccttagctaagagcagtacgagtgatgcagcaaaagctaatgatgagttagaaattaaagcaacagatccgactaattatccaaactgtggcgatgtgtatgggccattatttgaattggatgctagcggacagcttgttaataaagatgaagttatatctcttaaagatatgtatattttccaaatattgaaattagtaaatacaaaagatagtgactttcaatatgtaatattaacaatgaatcgtaaagatactgcagataggtctgtatatctttttgtaactggaagcaattatagtaatgctgttgttgttaaagtaaagccaaatgatacttatgaattaagtaaaactggatatagtgttacttatacagaaccaacaactataaatggacattatgttgatggaactttttatgttacaggaagtacttacgatgatggttttataatgccagattggcaactgcagcaccttcagattatatatagtttaggaaattatgatccaagcaatactgacgcaacatcagtttgtgaaataatgccaagttatgaaaaggtaccggtaattaaatatagtggagtaccttcaaatattagccaacctaaggtttacattaccgggtttacgggtcaagagtttaacgttacagatattattaacaattataagaaagtttttaagggctactatcttcaaaatcctaatgtggcgtccatgggaactctttcccaatttgagaatggtggttattacttaaagacatattatgataatgatggtaatgttgactttaagggcttgtatcatcaaattgatgatcagggaacaatgagtgtgagtgttcttaatgcagataataaaacaattgttggacctgaaaatattcttgctggtaaatcgcataactttaactttaatggtcataactggattgcgcggaatccttatgtcactagttcagctcacgaagtcatattaaagtatgctaagttaggttcagttattcctgttgatgaaaacggaaataaaataaacgatggatggcaatatgttaatgatccagatgatgcttccaaagccactagcccatatgaaaaagcgccagttatcgatggttatgtagctgtaaatccagatgaaacgatcgttcttcctcataacttaagtagtgacacaaagatttattaccgaaagaggattggtagtgctggtagtgctgaagctggtagtaattggagtcatccacaatttgaaaaaggtagtgctggtagtgctgctggtagtcacggttacgtatcggcagttgaaaacggtgtagccgaagggcgtgtaactctttgtaaatttgcagccaacggtacaggggagaaaaacacacactgtggtgcaattcaatatgaacctcaatctgtagaaggtcctgatggtttccctgtaacaggtcctcgtgatggtaaaattgcctctgcagaatctgcccttgcagccgcacttgatgaacaaactgcagaccgttgggtcaagcgtcctattcaagcaggtcctcaaactttcgagtggaccttcactgcaaaccacgtaacgaaggattggaagtactacattactaagccaaactggaacccaaaccagcctcttagccgtgatgcatttgacttgaaccctttctgtgtcgtagaagggaacatggttcagcctcctaagcgtgtatctcacgaatgtattgttcctgaacgtgaagggtaccaggtaatcctagcagtctgggatgtaggtgatactgcagcctcgttctacaacgttattgacgttaagtttgacggtggtagtgctggtagtgctgctggtagtggtgaatttaaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgtgactgtaaattacaagggctatgataagaaagtctatgatggtcaacctggaacgattaatccgggtaaattaacgtggagtaagttgccagatggtacttcattgaagatgccaacatggagtatagatgatttcgcttgggaaacagctgatggcttagcaccaacggcagtaggaacttatcggattatcttgacggatgctggtaaggctgcactaaagaagattaatccaaattatgacttaagcagtattactggtgtctttacttatgaaattaagccagcacagacaccagaaatcttaggccaaacacctgagcaacaaccaggccaaaatactaatcaatcaggagctgaaaacggctttggttcttctacaaggcctaatgcatcaactaactccaatcttaatcaacttccacagactggtaatgagcattctaatactgcacttgctggtctagcattggctttcttgactgctatgcttggtttgggcaagaagcgtaaacatgattag

SEQ ID NO: 31 - DNA, 4991 bp, Salmonella enterica
  source 1..4991: mol_type = genomic DNA
cccaatatccctgtcaattatgttgttttagatcaacaacaagccgggtatgtggttaaccacaatagagcgcaccccgcctcgatttttacactgtaaatcatcgacattttttattcattacacatgaaccaacatcgtgacaaatgtttcattgttggcaatgtggacgggagtcaatatggacagcagtaaacggcaatttctccagcagcttggcgtcctgaccgctggcgcctcgctggttccgctggctgaagcgaaatttcctttttcgccggagcggcatgaaggctctccccgacaccgttacgccatgcttatcgatctgcggcgttgtatcggctgtcagtcctgtaccgtaagttgcactattgaaaaccaaacgccgcaaggcgcgtttcgtacgacggtgaaccaataccaggtccagcgtgaaggtagtcaggaagtcacgaatgtgctgttgccgcgtctgtgcaaccattgcgataaccccccctgtgtgccggtctgcccggtacaagccacctttcagcgggaagatggcattgtggtggtggataacaaacgctgcgtcggctgcgcctattgtgtccaggcgtgtccttacgacgcccgatttatcaatcatgaaacgcaaactgccgataaatgcacgttttgcgtccatcgtctggaagccggactgttacccgcttgcgtagagtcctgcgtcggcggcgcgcgtattattggcgatatcaaagatccccatagccgcatcgccaccatgcttcatcagcatcgcgacgctatcaaggtattaaagccggaaaacggcacgtcgccccatgttttctacctgggtctggacgacgcctttgtcaccccattaatgggccgtgcgcagcccgcgctttggcaggaggtctgaatgacgcattcactcatcattgaagaagtgctggctcacccgcaggacattagctggctgccgtgggcggtacaatatttcttttttattggcattgccgcctgcgccgcactgtttgcctgttatcttcactggcggaaaaaagacgccgcaacagaagaaaatcgggcattactgattgccattacctgtgcgattaccgcaccgctggcgctgacggcggatctgcaccagaccgcccgcgtctggcatttctatgcctggccgacgccctggtcgtggatgccctggggagcgttattcctgccgctgtttaccggatttctcgctctgtggttcctggcgcagcagattaaacgattattcaataaaagttacaacgtcactaaatggttggcgttagccagcgcgctttgcgcggtgggcctgttgatttataccggccgcgaagtctccgttgtgctggcgcgcccaatctggtttagctacgccttccccgtggcgatgtttcttagcgccttacaggcgttcttcgcgctgatgattgtcgccgcccgacgcgactcggtaaggctgccaaaaatattgtggggacaaatctggacgctggcggcgctggggctggttgtggccatgtgggttagcggcgatacgctttccggcacggcaatccgtcagtggattaccgtcgccctgtcagccaaatattacgctgtcggctgggtagcgctgtgggtatgcacactgctgttctgtagcctggcgctacgccatccgttatcacagctaagacgcgtcctgctggttctcagcgcgctggcgctatgttggctgatgcgctggacattgttgattcaggtacaaaccgtccccaagttcaacgcgcaatttaacccttactcgttaccaggcggaacggatggctggctggctattctcggcaccttcggcctgtggatagcgctactgattattattcgtgaaacgctgaacggactcaccaggagattacaacatggctaatttaacccgtcgtcagtggctaaaagtcggtctcgccgtcggtgggatggtcacttttggtctgagctaccgtgatgtggcgaaacgcgcaattgatggcctgttaaacgggacgtccggcaaggtaacgcgcgaccgcatctttggcaatgcgttaattccggaggcgcaggcgcaaacacactggcagcaaaatccacaacaaaccatcgccatgacgcaatgcttcggctgttggacacagtgcggtatccgcgcccgggttaatgccgatggcaaagtgatacgcatcgccggcaatccctatcaccccttgtcgcaggaacacccgattgactcgtccgtcccttttagcgaagccatggagcaactggcgggagaaagcggtcttgacgcccgctcaaccgcctgcgcgcgcggcgccacgctgctggaaagcctgtacagtccgctacgactgcttgaaccgatgaaacgcgtgggtaaacgcggcgaagggaaatggcagcgcatcagctttgagcaacttattgaagaagtcgtggaaggcggcgatctgtttggcgaaggtcatgtggacggactgcgcgctattcatgcgccggatacgccaattgacgcaaagcaccccagtttcgggcccaaaaccaatcagttactggtcacgaataccagcgacgaaggccgcgatgcgtttctgcgtcgttttgcgctaaatagcttcggcagcaagaatttcggcgcgcatggcgcctactgtggactggcttaccgggccggctccggggcattgatgggcgatctggataaaaacccgcatgtcaaacccgactgggaaaacgtggagtttgcgctctttatgggcacctccccggcacagtccggcaatccgtttaaacgccaggcacgtcagttggcgagcgcccgactgcgtgagaattttcaatacgtcgtggtcgcccccgccctccccttatcaacggtgctcgccgatcctcgcggtcgctggcaaccggtcatgcccggcagtgattcggcgctggcaatggggatgatccgctggatcatggataatcaacgttataatgctgattatctggcgattcccggcgtacaggcgatgcagcaggccggcgagcaaagttggaccaacgccacgcacctggtcattgcggatgagctgccgacgcttgccggacaacacctgacgctgcgccatcttacgcccgatggcgaagagacccctgtcgtactgaataccgacggcgagttggtcgatgcgtccacttgccgacaggcacggcttttcgtgacgcagtacgttacgctcgccgacggccaacgggtcacggtgaagagcgggttgcaacgcctgaaagaggcggcagaaaagctctcgttggcgcaatacagcgaacagtgcggcgtgccggaagcgcaaattatcgcgctggcggaaacctttaccagtcacggacgtaaagctgcggtcatcagtcacggcggcatgatggccggcaatgggttttataacgcctggtcggtcatgatgcttaacgcgctgatcggcaacctcagcttgtccggcggcgtctttgtcggcggcggcaaattcaacggcgttagcgacggcccccgctacaacatgaacagttttgccggaaaagtgaaaccgtccgggttaagtattgcccgtagcaaaaccgcttatgaagcatcggaagaataccgcgacaaaattgccggtgggcaatccccttatccagccaaagcgccgtggtatccctttgtggcaggccagcttaccgaactgttgacctccgcgctcgaaggctatccttatccgcttaaagcctggatttccaatatgagcaacccgttttacggtgttcccggtctacgcgccgtggcggaagaaaaactaaaagaccctcgccgactgccgctctttatcgcgattgacgcctttatgaatgaaacgacggcgctggcggattacattgtgccggatacgcacaattttgagagctggggctttacggcgccctggggcggcgtagccagtaaagccactaccgcccgctggccggttgtcgcccccgccactcaccgcacggcggacgggcaacctgtctcaatggaagcattttgtattgcggtagcaaaacggctccatctgcccggcttcggcgaccgggcgataaccgatccgcagggcaatacttttccactgaaccgggcggaagacttctatctgcgcgtagccgctaatatcgcctttatgggcaagacgccggtcgcgctggcaaatcaggaagatatttcgcttaccggcgtcagccgcattctgccagcaattcagcacacgcttaaagctgatgaggtcggtcgcgtggcgtttatctactcgcgtggcggccggtttgcgcccgaggatagcggctatacggagcaacggttaggtaacgcgtggaaaaaacccttacagatctggaatgcagatgtcgccgcccaccgtcacgccatcaccggggagcgcttcagcggttgcccggtctggtatccggcgcgtttgtcagatggtcgtgcgattgacgaccagtttcccattgggcaatggccgctgaaactgatttcatttaaatcaaataccatgtccagctcaacagccgtcatcccgcgcttacaccatgtgaagccagcaaacctggtggcgctgaatccgcaagacggcgagcgttatggactgcaacatggcgatcgggtacggatcattacgccgggcggtcaggtcgtggcgcaaatcagtttgttaaatggcgtgatgccaggcgtcatcgccatcgaacacggatatggccaccgcgagatgggcgcaacgcagcactctctggatggcgtgcctatgccgtatgatccacaaatcagggcaggcataaatcttaacgatctgggctttgccgatccgacaagaaccattaccaacacctggctcgactgggtttctggcgcggcagtacgtcaggggctgccggcaaaaatcgagcgtata

SEQ ID NO: 32 - DNA, 2374 bp, Salmonella enterica
  source 1..2374: mol_type = genomic DNA
tcatggctcatacgttgttcgtattctggtctctggcgaggccattttttcgaaacgcctaatcagttccgccaggctaccggcctgcattttttccatgactctggcgcggtgcacctctacggtacgcaccgcgatattcatcgcttccgcaatttcacggttcataaatccttttgccaccaggctggccagctcacgctctttcggcgtcaactgctggtaacacagtataatctcacgacgcgccaccgctgccgatgaaaccgtcagcgcacgctccagcgccgcctgtagcggttttaccgataccggtttttgcagaaaatcgacggcgccgcgtttcatctgctccacggccatcggtacatcgccatgcccggtaagaaaaacaaccgccagggtacttccgcactggcgcaacgcatcatgaacgccctgcccatccagtaccggcattcgcatatccagtaatacgaccccggcctgatacagactggcctgcgccaaaaaatccgccccctgcgtccagcattttacgtcatatcccagactttccagtaaaaacgcgcacgcgttagtgaccgccgtatcatcatccagtagatgaattgtcgccatccctgcccccattttcatgtaagaaatgtatcgtaaccaccgttcccgacagaccgtccggcgcggtctggttcctgatgctgatatcgccccgcccataccgcaccagccgctggcaaatcgccagccctaagcccatcccctctttacgggtggtcataaacggctgaaacgcctgacgtaatagcgcctcatcgattcccccggcgttatcctgtaaaacaatactgatgccgttttcagtgcgttcagcaacgatccataaatgggtggcgcccgcctgagccgcattaagaatgatattcgccagcacctgttccagcagcactgacggcagcgttacgcgcagcgcagcgctaacctcggtatgcagagtcactgtcggaaactgttgcgccatacgcaacaattgccagacatgatcaatcgcctcgcgaatggctatggccttccacgcttcggttagcaccgggttgccctgcgcctggctgacccagtgacgcaggttacgcagagtatccgcaccgcgttgcgcctgctggtcaatctgctccagcgccggcagcaagggatgctgttcatctgcagcgcgcagtcgaatcaggcacccctgggcataatgtcgaatcgcggaaagcggctgattaagctcatgggcaaacccggaggtcatttcacccaacacgctcatttgccgggcggtttccagcgcccgctcatgctgatgaagaactacgctattacgttccagttgctttccacgtcgacgcaccagcagcatgacccaaatataattgagcgtgagcaacaagaacgccagaatcacgccgccgaccattagctggtgctggattaaccaacttttgacatccagccacagtcgacgctgctgagggtgctgacgaacatcacgcagcaaggcttccacctgactggtggacgcaggcgcgccccagtgaaatgacgcggcggcgggcgcgttgaatagcgctcgcgttacgcgatccgccagcgcatcgcttaccgcaggtagcgccgcgaacgaccagtcaggatataacggcgtactggttaagcaaggcaggggcgtcggtcgggaaagcagcgcgataaagtcctttttattaatcaatccttcctgatccatattttctaacaggcacactggcacaattgccgcctgcaccgctttttcgcgcagcatatagactaaggcatcgccaggaaatccggtaaaacggagatgaaaatcgcgctccgggcgtaagcccgcgtcgctgagcgctttatagcctaataaatagccgccaaacgcctgagcatcaatcgcgccgacggtcttaccgatgagatcatgcgccgtggtgatgccgctatcgcgccgggtcaaaatcacgctgccaataacattactcaccgctttcccatcgcgcgtggagcgcagggaagctaaccagcgcagcggcgcatggctgttcagttggacaaattgcgccgggttggttatcacaaactgcacggttccctggttaacggcctcctgcatttgatgcagatccagcggctggatgtgaaaggtttcgcctggaagctgttggcttaatgtctttgccaacggttgccagtggctacgcgtagacgcctcgccgcgcatggccaaaataccgatattccacgtccctgcccacgcgccatgacaaagtagccctactgccgccaacaccgccaggcgccttacggttttacctctcac

SEQ ID NO: 33 - DNA, 3060 bp, Salmonella enterica
  source 1..3060: mol_type = genomic DNA
atggctaatttaacccgtcgtcagtggctaaaagtcggtctcgccgtcggtgggatggtcacttttggtctgagctaccgtgatgtggcgaaacgcgcaattgatggcctgttaaacgggacgtccggcaaggtaacgcgcgaccgcatctttggcaatgcgttaattccggaggcgcaggcgcaaacacactggcagcaaaatccacaacaaaccatcgccatgacgcaatgcttcggctgttggacacagtgcggtatccgcgcccgggttaatgccgatggcaaagtgatacgcatcgccggcaatccctatcaccccttgtcgcaggaacacccgattgactcgtccgtcccttttagcgaagccatggagcaactggcgggagaaagcggtcttgacgcccgctcaaccgcctgcgcgcgcggcgccacgctgctggaaagcctgtacagtccgctacgactgcttgaaccgatgaaacgcgtgggtaaacgcggcgaagggaaatggcagcgcatcagctttgagcaacttattgaagaagtcgtggaaggcggcgatctgtttggcgaaggtcatgtggacggactgcgcgctattcatgcgccggatacgccaattgacgcaaagcaccccagtttcgggcccaaaaccaatcagttactggtcacgaataccagcgacgaaggccgcgatgcgtttctgcgtcgttttgcgctaaatagcttcggcagcaagaatttcggcgcgcatggcgcctactgtggactggcttaccgggccggctccggggcattgatgggcgatctggataaaaacccgcatgtcaaacccgactgggaaaacgtggagtttgcgctctttatgggcacctccccggcacagtccggcaatccgtttaaacgccaggcacgtcagttggcgagcgcccgactgcgtgagaattttcaatacgtcgtggtcgcccccgccctccccttatcaacggtgctcgccgatcctcgcggtcgctggcaaccggtcatgcccggcagtgattcggcgctggcaatggggatgatccgctggatcatggataatcaacgttataatgctgattatctggcgattcccggcgtacaggcgatgcagcaggccggcgagcaaagttggaccaacgccacgcacctggtcattgcggatgagctgccgacgcttgccggacaacacctgacgctgcgccatcttacgcccgatggcgaagagacccctgtcgtactgaataccgacggcgagttggtcgatgcgtccacttgccgacaggcacggcttttcgtgacgcagtacgttacgctcgccgacggccaacgggtcacggtgaagagcgggttgcaacgcctgaaagaggcggcagaaaagctctcgttggcgcaatacagcgaacagtgcggcgtgccggaagcgcaaattatcgcgctggcggaaacctttaccagtcacggacgtaaagctgcggtcatcagtcacggcggcatgatggccggcaatgggttttataacgcctggtcggtcatgatgcttaacgcgctgatcggcaacctcagcttgtccggcggcgtctttgtcggcggcggcaaattcaacggcgttagcgacggcccccgctacaacatgaacagttttgccggaaaagtgaaaccgtccgggttaagtattgcccgtagcaaaaccgcttatgaagcatcggaagaataccgcgacaaaattgccggtgggcaatccccttatccagccaaagcgccgtggtatccctttgtggcaggccagcttaccgaactgttgacctccgcgctcgaaggctatccttatccgcttaaagcctggatttccaatatgagcaacccgttttacggtgttcccggtctacgcgccgtggcggaagaaaaactaaaagaccctcgccgactgccgctctttatcgcgattgacgcctttatgaatgaaacgacggcgctggcggattacattgtgccggatacgcacaattttgagagctggggctttacggcgccctggggcggcgtagccagtaaagccactaccgcccgctggccggttgtcgcccccgccactcaccgcacggcggacgggcaacctgtctcaatggaagcattttgtattgcggtagcaaaacggctccatctgcccggcttcggcgaccgggcgataaccgatccgcagggcaatacttttccactgaaccgggcggaagacttctatctgcgcgtagccgctaatatcgcctttatgggcaagacgccggtcgcgctggcaaatcaggaagatatttcgcttaccggcgtcagccgcattctgccagcaattcagcacacgcttaaagctgatgaggtcggtcgcgtggcgtttatctactcgcgtggcggccggtttgcgcccgaggatagcggctatacggagcaacggttaggtaacgcgtggaaaaaacccttacagatctggaatgcagatgtcgccgcccaccgtcacgccatcaccggggagcgcttcagcggttgcccggtctggtatccggcgcgtttgtcagatggtcgtgcgattgacgaccagtttcccattgggcaatggccgctgaaactgatttcatttaaatcaaataccatgtccagctcaacagccgtcatcccgcgcttacaccatgtgaagccagcaaacctggtggcgctgaatccgcaagacggcgagcgttatggactgcaacatggcgatcgggtacggatcattacgccgggcggtcaggtcgtggcgcaaatcagtttgttaaatggcgtgatgccaggcgtcatcgccatcgaacacggatatggccaccgcgagatgggcgcaacgcagcactctctggatggcgtgcctatgccgtatgatccacaaatcagggcaggcataaatcttaacgatctgggctttgccgatccgacaagaaccattaccaacacctggctcgactgggtttctggcgcggcagtacgtcaggggctgccggcaaaaatcgagcgtata

SEQ ID NO: 34 - protein, 1020 aa, Salmonella enterica
MANLTRRQWLKVGLAVGGMVTFGLSYRDVAKRAIDGLLNGTSGKVTRDRIFGNALIPEAQAQTHWQQNPQQTIAMTQCFGCWTQCGIRARVNADGKVIRIAGNPYHPLSQEHPIDSSVPFSEAMEQLAGESGLDARSTACARGATLLESLYSPLRLLEPMKRVGKRGEGKWQRISFEQLIEEVVEGGDLFGEGHVDGLRAIHAPDTPIDAKHPSFGPKTNQLLVTNTSDEGRDAFLRRFALNSFGSKNFGAHGAYCGLAYRAGSGALMGDLDKNPHVKPDWENVEFALFMGTSPAQSGNPFKRQARQLASARLRENFQYVVVAPALPLSTVLADPRGRWQPVMPGSDSALAMGMIRWIMDNQRYNADYLAIPGVQAMQQAGEQSWTNATHLVIADELPTLAGQHLTLRHLTPDGEETPVVLNTDGELVDASTCRQARLFVTQYVTLADGQRVTVKSGLQRLKEAAEKLSLAQYSEQCGVPEAQIIALAETFTSHGRKAAVISHGGMMAGNGFYNAWSVMMLNALIGNLSLSGGVFVGGGKFNGVSDGPRYNMNSFAGKVKPSGLSIARSKTAYEASEEYRDKIAGGQSPYPAKAPWYPFVAGQLTELLTSALEGYPYPLKAWISNMSNPFYGVPGLRAVAEEKLKDPRRLPLFIAIDAFMNETTALADYIVPDTHNFESWGFTAPWGGVASKATTARWPVVAPATHRTADGQPVSMEAFCIAVAKRLHLPGFGDRAITDPQGNTFPLNRAEDFYLRVAANIAFMGKTPVALANQEDISLTGVSRILPAIQHTLKADEVGRVAFIYSRGGRFAPEDSGYTEQRLGNAWKKPLQIWNADVAAHRHAITGERFSGCPVWYPARLSDGRAIDDQFPIGQWPLKLISFKSNTMSSSTAVIPRLHHVKPANLVALNPQDGERYGLQHGDRVRIITPGGQVVAQISLLNGVMPGVIAIEHGYGHREMGATQHSLDGVPMPYDPQIRAGINLNDLGFADPTRTITNTWLDWVSGAAVRQGLPAKIERI

SEQ ID NO: 35 - DNA, 752 bp, Salmonella enterica
  source 1..752: mol_type = genomic DNA
atgtggacgggagtcaatatggacagcagtaaacggcaatttctccagcagcttggcgtcctgaccgctggcgcctcgctggttccgctggctgaagcgaaatttcctttttcgccggagcggcatgaaggctctccccgacaccgttacgccatgcttatcgatctgcggcgttgtatcggctgtcagtcctgtaccgtaagttgcactattgaaaaccaaacgccgcaaggcgcgtttcgtacgacggtgaaccaataccaggtccagcgtgaaggtagtcaggaagtcacgaatgtgctgttgccgcgtctgtgcaaccattgcgataaccccccctgtgtgccggtctgcccggtacaagccacctttcagcgggaagatggcattgtggtggtggataacaaacgctgcgtcggctgcgcctattgtgtccaggcgtgtccttacgacgcccgatttatcaatcatgaaacgcaaactgccgataaatgcacgttttgcgtccatcgtctggaagccggactgttacccgcttgcgtagagtcctgcgtcggcggcgcgcgtattattggcgatatcaaagatccccatagccgcatcgccaccatgcttcatcagcatcgcgacgctatcaaggtattaaagccggaaaacggcacgtcgccccatgttttctacctgggtctggacgacgcctttgtcaccccattaatgggccgtgcgcagcccgcgctttggcaggaggtctg

SEQ ID NO: 36 - protein, 250 aa, Salmonella enterica
MWTGVNMDSSKRQFLQQLGVLTAGASLVPLAEAKFPFSPERHEGSPRHRYAMLIDLRRCIGCQSCTVSCTIENQTPQGAFRTTVNQYQVQREGSQEVTNVLLPRLCNHCDNPPCVPVCPVQATFQREDGIVVVDNKRCVGCAYCVQACPYDARFINHETQTADKCTFCVHRLEAGLLPACVESCVGGARIIGDIKDPHSRIATMLHQHRDAIKVLKPENGTSPHVFYLGLDDAFVTPLMGRAQPALWQEV

SEQ ID NO: 37 - DNA, 1019 bp, Salmonella enterica
  source 1..1019: mol_type = genomic DNA
atgacgcattcactcatcattgaagaagtgctggctcacccgcaggacattagctggctgccgtgggcggtacaatatttcttttttattggcattgccgcctgcgccgcactgtttgcctgttatcttcactggcggaaaaaagacgccgcaacagaagaaaatcgggcattactgattgccattacctgtgcgattaccgcaccgctggcgctgacggcggatctgcaccagaccgcccgcgtctggcatttctatgcctggccgacgccctggtcgtggatgccctggggagcgttattcctgccgctgtttaccggatttctcgctctgtggttcctggcgcagcagattaaacgattattcaataaaagttacaacgtcactaaatggttggcgttagccagcgcgctttgcgcggtgggcctgttgatttataccggccgcgaagtctccgttgtgctggcgcgcccaatctggtttagctacgccttccccgtggcgatgtttcttagcgccttacaggcgttcttcgcgctgatgattgtcgccgcccgacgcgactcggtaaggctgccaaaaatattgtggggacaaatctggacgctggcggcgctggggctggttgtggccatgtgggttagcggcgatacgctttccggcacggcaatccgtcagtggattaccgtcgccctgtcagccaaatattacgctgtcggctgggtagcgctgtgggtatgcacactgctgttctgtagcctggcgctacgccatccgttatcacagctaagacgcgtcctgctggttctcagcgcgctggcgctatgttggctgatgcgctggacattgttgattcaggtacaaaccgtccccaagttcaacgcgcaatttaacccttactcgttaccaggcggaacggatggctggctggctattctcggcaccttcggcctgtggatagcgctactgattattattcgtgaaacgctgaacggactcaccaggagattacaacatgg

SEQ ID NO: 38 - protein, 340 aa, Salmonella enterica
MTHSLIIEEVLAHPQDISWLPWAVQYFFFIGIAACAALFACYLHWRKKDAATEENRALLIAITCAITAPLALTADLHQTARVWHFYAWPTPWSWMPWGALFLPLFTGFLALWFLAQQIKRLFNKSYNVTKWLALASALCAVGLLIYTGREVSVVLARPIWFSYAFPVAMFLSALQAFFALMIVAARHDSVRLPKILWGQIWTLAALGLVVAMWVSGDTLSGTAIRQWITVALSAKYYAVGWVALWVCTLLFCSLALRHPLSQLRRVLLVLSALALCWLMRWTLLIQVQTVPKFNAQFNPYSLPGGTDGWLAILGTFGLWIALLIIIRETLNGLTRRLQHG

SEQ ID NO: 39 - DNA, 639 bp, Salmonella enterica
  source 1..639: mol_type = genomic DNA
atgaaaatgggggcagggatggcgacaattcatctactggatgatgatacggcggtcactaacgcgtgcgcgtttttactggaaagtctgggatatgacgtaaaatgctggacgcagggggcggattttttggcgcaggccagtctgtatcaggccggggtcgtattactggatatgcgaatgccggtactggatgggcagggcgttcatgatgcgttgcgccagtgcggaagtaccctggcggttgtttttcttaccgggcatggcgatgtaccgatggccgtggagcagatgaaacgcggcgccgtcgattttctgcaaaaaccggtatcggtaaaaccgctacaggcggcgctggagcgtgcgctgacggtttcatcggcagcggtggcgcgtcgtgagattatactgtgttaccagcagttgacgccgaaagagcgtgagctggccagcctggtggcaaaaggatttatgaaccgtgaaattgcggaagcgatgaatatcgcggtgcgtaccgtagaggtgcaccgcgccagagtcatggaaaaaatgcaggccggtagcctggcggaactgattaggcgtttcgaaaaaatggcctcgccagagaccagaatacgaacaacgtatgagccatga

SEQ ID NO: 40 - protein, 212 aa, Salmonella enterica
MKMGAGMATIHLLDDDTAVTNACAFLLESLGYDVKCWTQGADFLAQASLYQAGVVLLDMRMPVLDGQGVHDALRQCGSTLAVVFLTGHGDVPMAVEQMKRGAVDFLQKPVSVKPLQAALERALTVSSAAVARREIILCYQQLTPKERELASLVAKGFMNREIAEAMNIAVRTVEVHRARVMEKMQAGSLAELIRRFEKMASPETRIRTTYEP

SEQ ID NO: 41 - DNA, 1779 bp, Salmonella enterica
  source 1..1779: mol_type = genomic DNA
gtgagaggtaaaaccgtaaggcgcctggcggtgttggcggcagtagggctactttgtcatggcgcgtgggcagggacgtggaatatcggtattttggccatgcgcggcgaggcgtctacgcgtagccactggcaaccgttggcaaagacattaagccaacagcttccaggcgaaacctttcacatccagccgctggatctgcatcaaatgcaggaggccgttaaccagggaaccgtgcagtttgtgataaccaacccggcgcaatttgtccaactgaacagccatgcgccgctgcgctggttagcttccctgcgctccacgcgcgatgggaaagcggtgagtaatgttattggcagcgtgattttgacccggcgcgatagcggcatcaccacggcgcatgatctcatcggtaagaccgtcggcgcgattgatgctcaggcgtttggcggctatttattaggctataaagcgctcagcgacgcgggcttacgcccggagcgcgattttcatctccgttttaccggatttcctggcgatgccttagtctatatgctgcgcgaaaaagcggtgcaggcggcaattgtgccagtgtgcctgttagaaaatatggatcaggaaggattgattaataaaaaggactttatcgcgctgctttcccgaccgacgcccctgccttgcttaaccagtacgccgttatatcctgactggtcgttcgcggcgctacctgcggtaagcgatgcgctggcggatcgcgtaacgcgagcgctattcaacgcgcccgccgccgcgtcatttcactggggcgcgcctgcgtccaccagtcaggtggaagccttgctgcgtgatgttcgtcagcaccctcagcagcgtcgactgtggctggatgtcaaaagttggttaatccagcaccagctaatggtcggcggcgtgattctggcgttcttgttgctcacgctcaattatatttgggtcatgctgctggtgcgtcgacgtggaaagcaactggaacgtaatagcgtagttcttcatcagcatgagcgggcgctggaaaccgcccggcaaatgagcgtgttgggtgaaatgacctccgggtttgcccatgagcttaatcagccgctttccgcgattcgacattatgcccaggggtgcctgattcgactgcgcgctgcagatgaacagcatcccttgctgccggcgctggagcagattgaccagcaggcgcaacgcggtgcggatactctgcgtaacctgcgtcactgggtcagccaggcgcagggcaacccggtgctaaccgaagcgtggaaggccatagccattcgcgaggcgattgatcatgtctggcaattgttgcgtatggcgcaacagtttccgacagtgactctgcataccgaggttagcgctgcgctgcgcgtaacgctgccgtcagtgctgctggaacaggtgctggcgaatatcattcttaatgcggctcaggcgggcgccacccatttatggatcgttgctgaacgcactgaaaacggcatcagtattgttttacaggataacgccgggggaatcgatgaggcgctattacgtcaggcgtttcagccgtttatgaccacccgtaaagaggggatgggcttagggctggcgatttgccagcggctggtgcggtatgggcggggcgatatcagcatcaggaaccagaccgcgccggacggtctgtcgggaacggtggttacgatacatttcttacatgaaaatgggggcagggatggcgacaattcatctactggatga

SEQ ID NO: 42 - protein, 592 aa, Salmonella enterica
MRGKTVRRLAVLAAVGLLCHGAWAGTWNIGILAMRGEASTRSHWQPLAKTLSQQLPGETFHIQPLDLHQMQEAVNQGTVQFVITNPAQFVQLNSHAPLRWLASLRSTRDGKAVSNVIGSVILTRRDSGITTAHDLIGKTVGAIDAQAFGGYLLGYKALSDAGLRPERDFHLRFTGFPGDALVYMLREKAVQAAIVPVCLLENMDQEGLINKKDFIALLSRPTPLPCLTSTPLYPDWSFAALPAVSDALADRVTRALFNAPAAASFHWGAPASTSQVEALLRDVRQHPQQRRLWLDVKSWLIQHQLMVGGVILAFLLLTLNYIWVMLLVRRRGKQLERNSVVLHQHERALETARQMSVLGEMTSGFAHELNQPLSAIRHYAQGCLIRLRAADEQHPLLPALEQIDQQAQRGADTLRNLRHWVSQAQGNPVLTEAWKAIAIREAIDHVWQLLRMAQQFPTVTLHTEVSAALRVTLPSVLLEQVLANIILNAAQAGATHLWIVAERTENGISIVLQDNAGGIDEALLRQAFQPFMTTRKEGMGLGLAICQRLVRYGRGDISIRNQTAPDGLSGTVVTIHFLHENGGRDGDNSSTG

SEQ ID NO: 43 - DNA, 5234 bp, synthetic construct
  misc_feature 1..5234: note = vector
  source 1..5234: mol_type = other DNA
aggcacacgaaaaacaagttaagggatgcagtttatcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagtcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccgattttttattaaaacgtctcaaaatcgtttctgagacgttttagcgtttatttcgtttagttatcggcataatcgttaaaacaggcgttatcgtagcgtaaaagcccttgagcgtagcgtggctttgcagcgaagatgttgtctgttagattatgaaagccgatgactgaatgaaataataagcgcagcgcccttctatttcggttggaggaggctcaagggagtatgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttaaggggtttttcaatcgccaacgaatcgccaacgttttcgccaacgttttttataaatctatatttaagtagctttattgttgtttttatgattacaaagtgatacactaactttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaacaaaaaaaataagcgaaagctcgcgtttttagaaggatacgagttttcgctacttgtttttgataaggtaattatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaataatagtaatattatacaaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacaaagatattgtttcaacaaagaatgatgtactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagccccctgacgaaagtcgaagggggtttttattttggtttgatgttgcgattaatagcaatacaattgcaataaacaaaatgatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacgatgacctctaataattgttaatcatgttggttacgtatttattaacttctcctagtattagtaattatcatggctgtcatggcgcattaacggaataaagggtgtgcttaaatcgggccattttgcgtaataagaaaaaggattaattatgagcgaattgaattaataataaggtaatagatttacattagaaaatgaaaggggattttatgcgtgagaatgttacagtctatccctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccag

SEQ ID NO: 44 - DNA, 5234 bp, synthetic construct; identical to SEQ ID NO: 43
  misc_feature 1..5234: note = vector
  source 1..5234: mol_type = other DNA

SEQ ID NO: 45 - DNA, 4500 bp, synthetic construct
  misc_feature 1..4500: note = vector
  source 1..4500: mol_type = other DNA
ttccccttctctgaaaatcaacgggcaggtcactgacttgcccgtttttttatcccttctccacaccgttgagctcgaattctcatgtttgacagcttatcactgatcagtgaattaatggcgatgacgcatcctcacgataatatccgggtaggcgcaatcactttcgtctctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaagctagaggtgaatcacgacaaagcgtatcaaaaacgtatggagtagggctctaaactctgtataaaaagtttccagctagctgataacgggaaagaaacagagaagggcacaaatattgtgtactttaatgtgccctttaatttattgattggtggttgaattgtccgtaactttttgatttaagtgcaaatttctaataaattagaacactttcttaaattgtcatttggcatattacgaacaattccgcgtaaaaacgttctgttacgctaaacccttatccagcaggctttcaaggatgtaaaccataacactctgcgaactagtgttacattgcgtgtagctttgagtgggcaactttgtgtacacttttgtgtacccaaaaacaaaaatgtgtacccattcaatgatcaccgacacaaagctcaggaaggcgctcggcaagaaaagagatgatatcgagattatttctgattcgcacgagctttctagacgctcaagttagtataaaaaagctgaacgagaaacgtaaaatgatataaatatcaatatattaaattagattttgcataaaaaacagactacataatactgtaaaacacaacatatgcagtcactatgaatcaactacttagatggtattagtgacctgtaacagactgcgggcccaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaatgaatagttcgacaaagatcgcattggtaattacgttactcgatgccatggggattggccttatcatgccagtcttgccaacgttattacgtgaatttattgcttcggaagatatcgctaaccactttggcgtattgcttgcactttatgcgttaatgcaggttatctttgctccttggcttggaaaaatgtctgaccgatttggtcggcgcccagtgctgttgttgtcattaataggcgcatcgctggattacttattgctggctttttcaagtgcgctttggatgctgtatttaggccgtttgctttcagggatcacaggagctactggggctgtcgcggcatcggtcattgccgataccacctcagcttctcaacgcgtgaagtggttcggttggttaggggcaagttttgggcttggtttaatagcggggcctattattggtggttttgcaggagagatttcaccgcatagtcccttttttatcgctgcgttgctaaatattgtcactttccttgtggttatgttttggttccgtgaaaccaaaaatacacgtgataatacagataccgaagtaggggttgagacgcaatcgaattcggtatacatcactttatttaaaacgatgcccattttgttgattatttatttttcagcgcaattgataggccaaattcccgcaacggtgtgggtgctatttaccgaaaatcgttttggatggaatagcatgatggttggcttttcattagcgggtcttggtcttttacactcagtattccaagcctttgtggcaggaagaatagccactaaatggggcgaaaaaacggcagtactgctcgaatttattgcagatagtagtgcatttgcctttttagcgtttatatctgaaggttggttagatttccctgttttaattttattggctggtggtgggatcgctttacctgcattacagggagtgatgtctatccaaacaaagagtcatgagcaaggtgctttacagggattattggtgagccttaccaatgcaaccggtgttattggcccattactgtttactgttatttataatcattcactaccaatttgggatggctggatttggattattggtttagcgttttactgtattattatcctgctatcgatgaccttcatgttaacccctcaagctcaggggagtaaacaggagacaagtgcttagttatttcgtcaccaaatgatgttattccgcgaaatataatgaccctcttgataacccaagagggcattttttacgagacgtcctaattcccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcctctacgccggacgcatcgtggccggatcttgcggccgcaaaaattaaaaatgaagttttggaggcctcatttggtgacgaaataactaagcacttgtctcctgtttactcccctgagcttgaggggtcaacatgaaggtcattgatagcaggataataatacagtaaaacgctaaaccaataatccaaatccagccatcccaaattggtagtgaatgattataaataacagtaaacagtaatgggccaataacaccggttgcattggtaaggctcaccaataatccctgtaaagcaccttgctcatgactctttgtttggatagacatcactccctgtaatgcaggtaaagcgatcccaccaccagccaataaaattaaaacagggaaatctaaccaaccttcagatataaacgctaaaaaggcaaatgcactactatctgcaataaattcgagcagtactgccgttttttcgccccatttagtggctattcttcctgccacaaaggcttggaatactgagtgtaaaagaccaagacccgctaatgaaaagccaaccatcatgctattccatccaaaacgattttcggtaaatagcacccacaccgttgcgggaatttggcctatcaattcgaaatcaaataatgattttattttgactgatagtgacctgttcgttgcaacaaattgataagcaatgcttttttataatgccaacttagtataaaaaagcaggcttcagagcgatggcccccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgc

SEQ ID NO: 46 - DNA, 10255 bp, synthetic construct
  misc_feature 1..10255: note = vector
  source 1..10255: mol_type = other DNA
ctgcaggtcgactctagaggatccgttatatacgctcgatttttgccggcagcccctgacgtactgccgcgccagaaacccagtcgagccaggtgttggtaatggttcttgtcggatcggcaaagcccagatcgttaagatttatgcctgccctgatttgtggatcatacggcataggcacgccatccagagagtgctgcgttgcgcccatctcgcggtggccatatccgtgttcgatggcgatgacgcctggcatcacgccatttaacaaactgatttgcgccacgacctgaccgcccggcgtaatgatccgtacccgatcgccatgttgcagtccataacgctcgccgtcttgcggattcagcgccaccaggtttgctggcttcacatggtgtaagcgcgggatgacggctgttgagctggacatggtatttgatttaaatgaaatcagtttcagcggccattgcccaatgggaaactggtcgtcaatcgcacgaccatctgacaaacgcgccggataccagaccgggcaaccgctgaagcgctccccggtgatggcgtgacggtgggcggcgacatctgcattccagatctgtaagggttttttccacgcgttacctaaccgttgctccgtatagccgctatcctcgggcgcaaaccggccgccacgcgagtagataaacgccacgcgaccgacctcatcagctttaagcgtgtgctgaattgctggcagaatgcggctgacgccggtaagcgaaatatcttcctgatttgccagcgcgaccggcgtcttgcccataaaggcgatattagcggctacgcgcagatagaagtcttccgcccggttcagtggaaaagtattgccctgcggatcggttatcgcccggtcgccgaagccgggcagatggagccgttttgctaccgcaatacaaaatgcttccattgagacaggttgcccgtccgccgtgcggtgagtggcgggggcgacaaccggccagcgggcggtagtggctttactggctacgccgccccagggcgccgtaaagccccagctctcaaaattgtgcgtatccggcacaatgtaatccgccagcgccgtcgtttcattcataaaggcgtcaatcgcgataaagagcggcagtcggcgagggtcttttagtttttcttccgccacggcgcgtagaccgggaacaccgtaaaacgggttgctcatattggaaatccaggctttaagcggataaggatagccttcgagcgcggaggtcaacagttcggtaagctggcctgccacaaagggataccacggcgctttggctggataaggggattgcccaccggcaattttgtcgcggtattcttccgatgcttcataagcggttttgctacgggcaatacttaacccggacggtttcacttttccggcaaaactgttcatgttgtagcgggggccgtcgctaacgccgttgaatttgccgccgccgacaaagacgccgccggacaagctgaggttgccgatcagcgcgttaagcatcatgaccgaccaggcgttataaaacccattgccggccatcatgccgccgtgactgatgaccgcagctttacgtccgtgactggtaaaggtttccgccagcgcgataatttgcgcttccggcacgccgcactgttcgctgtattgcgccaacgagagcttttctgccgcctctttcaggcgttgcaacccgctcttcaccgtgacccgttggccgtcggcgagcgtaacgtactgcgtcacgaaaagccgtgcctgtcggcaagtggacgcatcgaccaactcgccgtcggtattcagtacgacaggggtctcttcgccatcgggcgtaagatggcgcagcgtcaggtgttgtccggcaagcgtcggcagctcatccgcaatgaccaggtgcgtggcgttggtccaactttgctcgccggcctgctgcatcgcctgtacgccgggaatcgccagataatcagcattataacgttgattatccatgatccagcggatcatccccattgccagcgccgaatcactgccgggcatgaccggttgccagcgaccgcgaggatcggcgagcaccgttgataaggggagggcgggggcgaccacgacgtattgaaaattctcacgcagtcgggcgctcgccaactgacgtgcctggcgtttaaacggattgccggactgtgccggggaggtgcccataaagagcgcaaactccacgttttcccagtcgggtttgacatgcgggtttttatccagatcgcccatcaatgccccggagccggcccggtaagccagtccacagtaggcgccatgcgcgccgaaattcttgctgccgaagctatttagcgcaaaacgacgcagaaacgcatcgcggccttcgtcgctggtattcgtgaccagtaactgattggttttgggcccgaaactggggtgctttgcgtcaattggcgtatccggcgcatgaatagcgcgcagtccgtccacatgaccttcgccaaacagatcgccgccttccacgacttcttcaataagttgctcaaagctgatgcgctgccatttcccttcgccgcgtttacccacgcgtttcatcggttcaagcagtcgtagcggactgtacaggctttccagcagcgtggcgccgcgcgcgcaggcggttgagcgggcgtcaagaccgctttctcccgccagttgctccatggcttcgctaaaagggacggacgagtcaatcgggtgttcctgcgacaaggggtgatagggattgccggcgatgcgtatcactttgccatcggcattaacccgggcgcggataccgcactgtgtccaacagccgaagcattgcgtcatggcgatggtttgttgtggattttgctgccagtgtgtttgcgcctgcgcctccggaattaacgcattgccaaagatgcggtcgcgcgttaccttgccggacgtcccgtttaacaggccatcaattgcgcgtttcgccacatcacggtagctcagaccaaaagtgaccatcccaccgacggcgagaccgacttttagccactgacgacgggttaaattagccatgttgtaatctcctggtgagtccgttcagcgtttcacgaataataatcagtagcgctatccacaggccgaaggtgccgagaatagccagccagccatccgttccgcctggtaacgagtaagggttaaattgcgcgttgaacttggggacggtttgtacctgaatcaacaatgtccagcgcatcagccaacatagcgccagcgcgctgagaaccagcaggacgcgtcttagctgtgataacggatggcgtagcgccaggctacagaacagcagtgtgcatacccacagcgctacccagccgacagcgtaatatttggctgacagggcgacggtaatccactgacggattgccgtgccggaaagcgtatcgccgctaacccacatggccacaaccagccccagcgccgccagcgtccagatttgtccccacaatatttttggcagccttaccgagtcgcgtcgggcggcgacaatcatcagcgcgaagaacgcctgtaaggcgctaagaaacatcgccacggggaaggcgtagctaaaccagattgggcgcgccagcacaacggagacttcgcggccggtataaatcaacaggcccaccgcgcaaagcgcgctggctaacgccaaccatttagtgacgttgtaacttttattgaataatcgtttaatctgctgcgccaggaaccacagagcgagaaatccggtaaacagcggcaggaataacgctccccagggcatccacgaccagggcgtcggccaggcatagaaatgccagacgcgggcggtctggtgcagatccgccgtcagcgccagcggtgcggtaatcgcacaggtaatggcaatcagtaatgcccgattttcttctgttgcggcgtcttttttccgccagtgaagataacaggcaaacagtgcggcgcaggcggcaatgccaataaaaaagaaatattgtaccgcccacggcagccagctaatgtcctgcgggtgagccagcacttcttcaatgatgagtgaatgcgtcattcagacctcctgccaaagcgcgggctgcgcacggcccattaatggggtgacaaaggcgtcgtccagacccaggtagaaaacatggggcgacgtgccgttttccggctttaataccttgatagcgtcgcgatgctgatgaagcatggtggcgatgcggctatggggatctttgatatcgccaataatacgcgcgccgccgacgcaggactctacgcaagcgggtaacagtccggcttccagacgatggacgcaaaacgtgcatttatcggcagtttgcgtttcatgattgataaatcgggcgtcgtaaggacacgcctggacacaataggcgcagccgacgcagcgtttgttatccaccaccacaatgccatcttcccgctgaaaggtggcttgtaccgggcagaccggcacacagggggggttatcgcaatggttgcacagacgcggcaacagcacattcgtgacttcctgactaccttcacgctggacctggtattggttcaccgtcgtacgaaacgcgccttgcggcgtttggttttcaatagtgcaacttacggtacaggactgacagccgatacaacgccgcagatcgataagcatggcgtaacggtgtcggggagagccttcatgccgctccggcgaaaaaggaaatttcgcttcagccagcggaaccagcgaggcgccagcggtcaggacgccaagctgctggagaaattgccgtttactgctgtccatattgactcccgtccacattgccaacaatgaaacatttgtcacgatgttggttcatgtgtaatgaataaaaaatgtcgatgatttacagtgtaaaaatcgaggcggggtgcgctctattgtggttaaccacatacccggcttgttgttgatctaaaacaacataattgacagggatattggggtgagaggtaaaaccgtaaggcgcctggcggtgttggcggcagtagggctactttgtcatggcgcgtgggcagggacgtggaatatcggtattttggccatgcgcggcgaggcgtctacgcgtagccactggcaaccgttggcaaagacattaagccaacagcttccaggcgaaacctttcacatccagccgctggatctgcatcaaatgcaggaggccgttaaccagggaaccgtgcagtttgtgataaccaacccggcgcaatttgtccaactgaacagccatgcgccgctgcgctggttagcttccctgcgctccacgcgcgatgggaaagcggtgagtaatgttattggcagcgtgattttgacccggcgcgatagcggcatcaccacggcgcatgatctcatcggtaagaccgtcggcgcgattgatgctcaggcgtttggcggctatttattaggctataaagcgctcagcgacgcgggcttacgcccggagcgcgattttcatctccgttttaccggatttcctggcgatgccttagtctatatgctgcgcgaaaaagcggtgcaggcggcaattgtgccagtgtgcctgttagaaaatatggatcaggaaggattgattaataaaaaggactttatcgcgctgctttcccgaccgacgcccctgccttgcttaaccagtacgccgttatatcctgactggtcgttcgcggcgctacctgcggtaagcgatgcgctggcggatcgcgtaacgcgagcgctattcaacgcgcccgccgccgcgtcatttcactggggcgcgcctgcgtccaccagtcaggtggaagccttgctgcgtgatgttcgtcagcaccctcagcagcgtcgactgtggctggatgtcaaaagttggttaatccagcaccagctaatggtcggcggcgtgattctggcgttcttgttgctcacgctcaattatatttgggtcatgctgctggtgcgtcgacgtggaaagcaactggaacgtaatagcgtagttcttcatcagcatgagcgggcgctggaaaccgcccggcaaatgagcgtgttgggtgaaatgacctccgggtttgcccatgagcttaatcagccgctttccgcgattcgacattatgcccaggggtgcctgattcgactgcgcgctgcagatgaacagcatcccttgctgccggcgctggagcagattgaccagcaggcgcaacgcggtgcggatactctgcgtaacctgcgtcactgggtcagccaggcgcagggcaacccggtgctaaccgaagcgtggaaggccatagccattcgcgaggcgattgatcatgtctggcaattgttgcgtatggcgcaacagtttccgacagtgactctgcataccgaggttagcgctgcgctgcgcgtaacgctgccgtcagtgctgctggaacaggtgctggcgaatatcattcttaatgcggctcaggcgggcgccacccatttatggatcgttgctgaacgcactgaaaacggcatcagtattgttttacaggataacgccgggggaatcgatgaggcgctattacgtcaggcgtttcagccgtttatgaccacccgtaaagaggggatgggcttagggctggcgatttgccagcggctggtgcggtatgggcggggcgatatcagcatcaggaaccagaccgcgccggacggtctgtcgggaacggtggttacgatacatttcttacatgaaaatgggggcagggatggcgacaattcatctactggatgatgatacggcggtcactaacgcgtgcgcgtttttactggaaagtctgggatatgacgtaaaatgctggacgcagggggcggattttttggcgcaggccagtctgtatcaggccggggtcgtattactggatatgcgaatgccggtactggatgggcagggcgttcatgatgcgttgcgccagtgcggaagtaccctggcggttgtttttcttaccgggcatggcgatgtaccgatggccgtggagcagatgaaacgcggcgccgtcgattttctgcaaaaaccggtatcggtaaaaccgctacaggcggcgctggagcgtgcgctgacggtttcatcggcagcggtggcgcgtcgtgagattatactgtgttaccagcagttgacgccgaaagagcgtgagctggccagcctggtggcaaaaggatttatgaaccgtgaaattgcggaagcgatgaatatcgcggtgcgtaccgtagaggtgcaccgcgccagagtcatggaaaaaatgcaggccggtagcctggcggaactgattaggcgtttcgaaaaaatggcctcgccagagaccagaatacgaacaacgtatgagccatgaataagagctcgaattctcatgtttgacagcttatcactgatcagtgaattaatggcgatgacgcatcctcacgataatatccgggtaggcgcaatcactttcgtctctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaagctagaggtgaatcacgacaaagcgtatcaaaaacgtatggagtagggctctaaactctgtataaaaagtttccagctagctgataacgggaaagaaacagagaagggcacaaatattgtgtactttaatgtgccctttaatttattgattggtggttgaattgtccgtaactttttgatttaagtgcaaatttctaataaattagaacactttcttaaattgtcatttggcatattacgaacaattccgcgtaaaaacgttctgttacgctaaacccttatccagcaggctttcaaggatgtaaaccataacactctgcgaactagtgttacattgcgtgtagctttgagtgggcaactttgtgtacacttttgtgtacccaaaaacaaaaatgtgtacccattcaatgatcaccgacacaaagctcaggaaggcgctcggcaagaaaagagatgatatcgagattatttctgattcgcacgggcccatggctaattcccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcctctacgccggacgcatcgtggccggatcttgcggccgcaaaaattaaaaatgaagttttggaggcctcatttggtgacgaaataactaagcacttgtctcctgtttactcccctgagcttgaggggtcaacatgaaggtcattgatagcaggataataatacagtaaaacgctaaaccaataatccaaatccagccatcccaaattggtagtgaatgattataaataacagtaaacagtaatgggccaataacaccggttgcattggtaaggctcaccaataatccctgtaaagcaccttgctcatgactctttgtttggatagacatcactccctgtaatgcaggtaaagcgatcccaccaccagccaataaaattaaaacagggaaatctaaccaaccttcagatataaacgctaaaaaggcaaatgcactactatctgcaataaattcgagcagtactgccgttttttcgccccatttagtggctattcttcctgccacaaaggcttggaatactgagtgtaaaagaccaagacccgctaatgaaaagccaaccatcatgctattccatccaaaacgattttcggtaaatagcacccacaccgttgcgggaatttggcctatcaattgcgctgaaaaataaataatcaacaaaatgggcatcgttttaaataaagtgatgtacaccgaatttgattgcgtctcaacccctacttcggtatctgtattatcacgtgtatttttggtttcacggaaccaaaacataaccacaaggaaagtgacaatatttagcaacgcagcgataaaaaagggactatgcggtgaaatctctcctgcaaaaccaccaataataggccccgctattaaaccaagcccaaaacttgcccctaaccaaccgaaccacttcacgcgttgagaagctgaggtggtatcggcaatgaccgatgccgcgacagccccagtagctcctgtgatccctgaaagcaaacggcctaaatacagcatccaaagcgcacttgaaaaagccagcaataagtaatccagcgatgcgcctattaatgacaacaacagcactgggcgccgaccaaatcggtcagacatttttccaagccaaggagcaaagataacctgcattaacgcataaagtgcaagcaatacgccaaagtggttagcgatatcttccgaagcaataaattcacgtaataacgttggcaagactggcatgataaggccaatccccatggcatcgagtaacgtaattaccaatgcgatctttgtcgaactattcatttcacttttctctatcactgatagggagtgggaaaataactctatcaatgatagggtgtcaaatcgatggcccccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgc

SEQ ID NO: 47 - DNA, 10465 bp, synthetic construct
  misc_feature 1..10465: note = vector
  source 1..10465: mol_type = other DNA
gacctataaggaaaggccaaacaagaacacggttgcaaaaaccgtgcccttaaatattgaatttctattcagaacactttcttaaattgtcatttggcatattacgaacaattccgcgtaaaaacgttctgttacgctaaacccttatccagcaggctttcaaggatgtaaaccataacactctgcgaactagtgttacattgcgtgtagctttgagtgggcaactttgtgtacacttttgtgtacccaaaaacaaaaatgtgtacccattcaatgatcaccgacacaaagctcaggaaggcgctcggcaagaaaagagatgatatcgagattatttctgattcgcacgggcccatggctaattcccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcctctacgccggacgcatcgtggccggatcttgcggccgcaaaaattaaaaatgaagttttggaggcctcatttggtgacgaaataactaagcacttgtctcctgtttactcccctgagcttgaggggtcaacatgaaggtcattgatagcaggataataatacagtaaaacgctaaaccaataatccaaatccagccatcccaaattggtagtgaatgattataaataacagtaaacagtaatgggccaataacaccggttgcattggtaaggctcaccaataatccctgtaaagcaccttgctcatgactctttgtttggatagacatcactccctgtaatgcaggtaaagcgatcccaccaccagccaataaaattaaaacagggaaatctaaccaaccttcagatataaacgctaaaaaggcaaatgcactactatctgcaataaattcgagcagtactgccgttttttcgccccatttagtggctattcttcctgccacaaaggcttggaatactgagtgtaaaagaccaagacccgctaatgaaaagccaaccatcatgctattccatccaaaacgattttcggtaaatagcacccacaccgttgcgggaatttggcctatcaattgcgctgaaaaataaataatcaacaaaatgggcatcgttttaaataaagtgatgtacaccgaatttgattgcgtctcaacccctacttcggtatctgtattatcacgtgtatttttggtttcacggaaccaaaacataaccacaaggaaagtgacaatatttagcaacgcagcgataaaaaagggactatgcggtgaaatctctcctgcaaaaccaccaataataggccccgctattaaaccaagcccaaaacttgcccctaaccaaccgaaccacttcacgcgttgagaagctgaggtggtatcggcaatgaccgatgccgcgacagccccagtagctcctgtgatccctgaaagcaaacggcctaaatacagcatccaaagcgcacttgaaaaagccagcaataagtaatccagcgatgcgcctattaatgacaacaacagcactgggcgccgaccaaatcggtcagacatttttccaagccaaggagcaaagataacctgcattaacgcataaagtgcaagcaatacgccaaagtggttagcgatatcttccgaagcaataaattcacgtaataacgttggcaagactggcatgataaggccaatccccatggcatcgagtaacgtaattaccaatgcgatctttgtcgaactattcatttcacttttctctatcactgatagggagtgggaaaataactctatcaatgatagggtgtcaaatcgatggcccccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcctgcaggtcgactctagaggatccgttatatacgctcgatttttgccggcagcccctgacgtactgccgcgccagaaacccagtcgagccaggtgttggtaatggttcttgtcggatcggcaaagcccagatcgttaagatttatgcctgccctgatttgtggatcatacggcataggcacgccatccagagagtgctgcgttgcgcccatctcgcggtggccatatccgtgttcgatggcgatgacgcctggcatcacgccatttaacaaactgatttgcgccacgacctgaccgcccggcgtaatgatccgtacccgatcgccatgttgcagtccataacgctcgccgtcttgcggattcagcgccaccaggtttgctggcttcacatggtgtaagcgcgggatgacggctgttgagctggacatggtatttgatttaaatgaaatcagtttcagcggccattgcccaatgggaaactggtcgtcaatcgcacgaccatctgacaaacgcgccggataccagaccgggcaaccgctgaagcgctccccggtgatggcgtgacggtgggcggcgacatctgcattccagatctgtaagggttttttccacgcgttacctaaccgttgctccgtatagccgctatcctcgggcgcaaaccggccgccacgcgagtagataaacgccacgcgaccgacctcatcagctttaagcgtgtgctgaattgctggcagaatgcggctgacgccggtaagcgaaatatcttcctgatttgccagcgcgaccggcgtcttgcccataaaggcgatattagcggctacgcgcagatagaagtcttccgcccggttcagtggaaaagtattgccctgcggatcggttatcgcccggtcgccgaagccgggcagatggagccgttttgctaccgcaatacaaaatgcttccattgagacaggttgcccgtccgccgtgcggtgagtggcgggggcgacaaccggccagcgggcggtagtggctttactggctacgccgccccagggcgccgtaaagccccagctctcaaaattgtgcgtatccggcacaatgtaatccgccagcgccgtcgtttcattcataaaggcgtcaatcgcgataaagagcggcagtcggcgagggtcttttagtttttcttccgccacggcgcgtagaccgggaacaccgtaaaacgggttgctcatattggaaatccaggctttaagcggataaggatagccttcgagcgcggaggtcaacagttcggtaagctggcctgccacaaagggataccacggcgctttggctggataaggggattgcccaccggcaattttgtcgcggtattcttccgatgcttcataagcggttttgctacgggcaatacttaacccggacggtttcacttttccggcaaaactgttcatgttgtagcgggggccgtcgctaacgccgttgaatttgccgccgccgacaaagacgccgccggacaagctgaggttgccgatcagcgcgttaagcatcatgaccgaccaggcgttataaaacccattgccggccatcatgccgccgtgactgatgaccgcagctttacgtccgtgactggtaaaggtttccgccagcgcgataatttgcgcttccggcacgccgcactgttcgctgtattgcgccaacgagagcttttctgccgcctctttcaggcgttgcaacccgctcttcaccgtgacccgttggccgtcggcgagcgtaacgtactgcgtcacgaaaagccgtgcctgtcggcaagtggacgcatcgaccaactcgccgtcggtattcagtacgacaggggtctcttcgccatcgggcgtaagatggcgcagcgtcaggtgttgtccggcaagcgtcggcagctcatccgcaatgaccaggtgcgtggcgttggtccaactttgctcgccggcctgctgcatcgcctgtacgccgggaatcgccagataatcagcattataacgttgattatccatgatccagcggatcatccccattgccagcgccgaatcactgccgggcatgaccggttgccagcgaccgcgaggatcggcgagcaccgttgataaggggagggcgggggcgaccacgacgtattgaaaattctcacgcagtcgggcgctcgccaactgacgtgcctggcgtttaaacggattgccggactgtgccggggaggtgcccataaagagcgcaaactccacgttttcccagtcgggtttgacatgcgggtttttatccagatcgcccatcaatgccccggagccggcccggtaagccagtccacagtaggcgccatgcgcgccgaaattcttgctgccgaagctatttagcgcaaaacgacgcagaaacgcatcgcggccttcgtcgctggtattcgtgaccagtaactgattggttttgggcccgaaactggggtgctttgcgtcaattggcgtatccggcgcatgaatagcgcgcagtccgtccacatgaccttcgccaaacagatcgccgccttccacgacttcttcaataagttgctcaaagctgatgcgctgccatttcccttcgccgcgtttacccacgcgtttcatcggttcaagcagtcgtagcggactgtacaggctttccagcagcgtggcgccgcgcgcgcaggcggttgagcgggcgtcaagaccgctttctcccgccagttgctccatggcttcgctaaaagggacggacgagtcaatcgggtgttcctgcgacaaggggtgatagggattgccggcgatgcgtatcactttgccatcggcattaacccgggcgcggataccgcactgtgtccaacagccgaagcattgcgtcatggcgatggtttgttgtggattttgctgccagtgtgtttgcgcctgcgcctccggaattaacgcattgccaaagatgcggtcgcgcgttaccttgccggacgtcccgtttaacaggccatcaattgcgcgtttcgccacatcacggtagctcagaccaaaagtgaccatcccaccgacggcgagaccgacttttagccactgacgacgggttaaattagccatgttgtaatctcctggtgagtccgttcagcgtttcacgaataataatcagtagcgctatccacaggccgaaggtgccgagaatagccagccagccatccgttccgcctggtaacgagtaagggttaaattgcgcgttgaacttggggacggtttgtacctgaatcaacaatgtccagcgcatcagccaacatagcgccagcgcgctgagaaccagcaggacgcgtcttagctgtgataacggatggcgtagcgccaggctacagaacagcagtgtgcatacccacagcgctacccagccgacagcgtaatatttggctgacagggcgacggtaatccactgacggattgccgtgccggaaagcgtatcgccgctaacccacatggccacaaccagccccagcgccgccagcgtccagatttgtccccacaatatttttggcagccttaccgagtcgcgtcgggcggcgacaatcatcagcgcgaagaacgcctgtaaggcgctaagaaacatcgccacggggaaggcgtagctaaaccagattgggcgcgccagcacaacggagacttcgcggccggtataaatcaacaggcccaccgcgcaaagcgcgctggctaacgccaaccatttagtgacgttgtaacttttattgaataatcgtttaatctgctgcgccaggaaccacagagcgagaaatccggtaaacagcggcaggaataacgctccccagggcatccacgaccagggcgtcggccaggcatagaaatgccagacgcgggcggtctggtgcagatccgccgtcagcgccagcggtgcggtaatcgcacaggtaatggcaatcagtaatgcccgattttcttctgttgcggcgtcttttttccgccagtgaagataacaggcaaacagtgcggcgcaggcggcaatgccaataaaaaagaaatattgtaccgcccacggcagccagctaatgtcctgcgggtgagccagcacttcttcaatgatgagtgaatgcgtcattcagacctcctgccaaagcgcgggctgcgcacggcccattaatggggtgacaaaggcgtcgtccagacccaggtagaaaacatggggcgacgtgccgttttccggctttaataccttgatagcgtcgcgatgctgatgaagcatggtggcgatgcggctatggggatctttgatatcgccaataatacgcgcgccgccgacgcaggactctacgcaagcgggtaacagtccggcttccagacgatggacgcaaaacgtgcatttatcggcagtttgcgtttcatgattgataaatcgggcgtcgtaaggacacgcctggacacaataggcgcagccgacgcagcgtttgttatccaccaccacaatgccatcttcccgctgaaaggtggcttgtaccgggcagaccggcacacagggggggttatcgcaatggttgcacagacgcggcaacagcacattcgtgacttcctgactaccttcacgctggacctggtattggttcaccgtcgtacgaaacgcgccttgcggcgtttggttttcaatagtgcaacttacggtacaggactgacagccgatacaacgccgcagatcgataagcatggcgtaacggtgtcggggagagccttcatgccgctccggcgaaaaaggaaatttcgcttcagccagcggaaccagcgaggcgccagcggtcaggacgccaagctgctggagaaattgccgtttactgctgtccatattgactcccgtccacattgccaacaatgaaacatttgtcacgatgttggttcatgtgtaatgaataaaaaatgtcgatgatttacagtgtaaaaatcgaggcggggtgcgctctattgtggttaaccacatacccggcttgttgttgatctaaaacaacataattgacagggatattggggtgagaggtaaaaccgtaaggcgcctggcggtgttggcggcagtagggctactttgtcatggcgcgtgggcagggacgtggaatatcggtattttggccatgcgcggcgaggcgtctacgcgtagccactggcaaccgttggcaaagacattaagccaacagcttccaggcgaaacctttcacatccagccgctggatctgcatcaaatgcaggaggccgttaaccagggaaccgtgcagtttgtgataaccaacccggcgcaatttgtccaactgaacagccatgcgccgctgcgctggttagcttccctgcgctccacgcgcgatgggaaagcggtgagtaatgttattggcagcgtgattttgacccggcgcgatagcggcatcaccacggcgcatgatctcatcggtaagaccgtcggcgcgattgatgctcaggcgtttggcggctatttattaggctataaagcgctcagcgacgcgggcttacgcccggagcgcgattttcatctccgttttaccggatttcctggcgatgccttagtctatatgctgcgcgaaaaagcggtgcaggcggcaattgtgccagtgtgcctgttagaaaatatggatcaggaaggattgattaataaaaaggactttatcgcgctgctttcccgaccgacgcccctgccttgcttaaccagtacgccgttatatcctgactggtcgttcgcggcgctacctgcggtaagcgatgcgctggcggatcgcgtaacgcgagcgctattcaacgcgcccgccgccgcgtcatttcactggggcgcgcctgcgtccaccagtcaggtggaagccttgctgcgtgatgttcgtcagcaccctcagcagcgtcgactgtggctggatgtcaaaagttggttaatccagcaccagctaatggtcggcggcgtgattctggcgttcttgttgctcacgctcaattatatttgggtcatgctgctggtgcgtcgacgtggaaagcaactggaacgtaatagcgtagttcttcatcagcatgagcgggcgctggaaaccgcccggcaaatgagcgtgttgggtgaaatgacctccgggtttgcccatgagcttaatcagccgctttccgcgattcgacattatgcccaggggtgcctgattcgactgcgcgctgcagatgaacagcatcccttgctgccggcgctggagcagattgaccagcaggcgcaacgcggtgcggatactctgcgtaacctgcgtcactgggtcagccaggcgcagggcaacccggtgctaaccgaagcgtggaaggccatagccattcgcgaggcgattgatcatgtctggcaattgttgcgtatggcgcaacagtttccgacagtgactctgcataccgaggttagcgctgcgctgcgcgtaacgctgccgtcagtgctgctggaacaggtgctggcgaatatcattcttaatgcggctcaggcgggcgccacccatttatggatcgttgctgaacgcactgaaaacggcatcagtattgttttacaggataacgccgggggaatcgatgaggcgctattacgtcaggcgtttcagccgtttatgaccacccgtaaagaggggatgggcttagggctggcgatttgccagcggctggtgcggtatgggcggggcgatatcagcatcaggaaccagaccgcgccggacggtctgtcgggaacggtggttacgatacatttcttacatgaaaatgggggcagggatggcgacaattcatctactggatgatgatacggcggtcactaacgcgtgcgcgtttttactggaaagtctgggatatgacgtaaaatgctggacgcagggggcggattttttggcgcaggccagtctgtatcaggccggggtcgtattactggatatgcgaatgccggtactggatgggcagggcgttcatgatgcgttgcgccagtgcggaagtaccctggcggttgtttttcttaccgggcatggcgatgtaccgatggccgtggagcagatgaaacgcggcgccgtcgattttctgcaaaaaccggtatcggtaaaaccgctacaggcggcgctggagcgtgcgctgacggtttcatcggcagcggtggcgcgtcgtgagattatactgtgttaccagcagttgacgccgaaagagcgtgagctggccagcctggtggcaaaaggatttatgaaccgtgaaattgcggaagcgatgaatatcgcggtgcgtaccgtagaggtgcaccgcgccagagtcatggaaaaaatgcaggccggtagcctggcggaactgattaggcgtttcgaaaaaatggcctcgccagagaccagaatacgaacaacgtatgagccatgaataagagctcgaattctcatgtttgacagcttatcactgatcagtgaattaatggcgatgacgcatcctcacgataatatccgggtaggcgcaatcactttcgtctctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaagctagaggtgaatcacgacaaagcgtatcaaaaacgtatggagtagggctctaaactctgtataaaaagtttccagctagctgataacgggaaagaaacagagaagggcacaaatattgtgtactttaatgtgccctttaatttattgattggtggttgaattgtccgtaactttttgatttaagtgcaaatttctaataaattagaacactttcttaaatggtttcactgaaacgtgttcatagactcctgccgctacgtacgggtcagcatcggcccaagcctgagctgcttccagcgactcaaattcagcaataacggttgagccagtaaatcccgcagcc

SEQ ID NO: 48 - DNA, 4395 bp, synthetic construct
  misc_feature 1..4395: note = vector
  source 1..4395: mol_type = other DNA
gacctataaggaaaggccaaacaagaacacggttgcaaaaaccgtgcccttaaatattgaatttctattcagaacactttcttaaattgtcatttggcatattacgaacaattccgcgtaaaaacgttctgttacgctaaacccttatccagcaggctttcaaggatgtaaaccataacactctgcgaactagtgttacattgcgtgtagctttgagtgggcaactttgtgtacacttttgtgtacccaaaaacaaaaatgtgtacccattcaatgatcaccgacacaaagctcaggaaggcgctcggcaagaaaagagatgatatcgagattatttctgattcgcacgggcccatggctaattcccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcctctacgccggacgcatcgtggccggatcttgcggccgcaaaaattaaaaatgaagttttggaggcctcatttggtgacgaaataactaagcacttgtctcctgtttactcccctgagcttgaggggtcaacatgaaggtcattgatagcaggataataatacagtaaaacgctaaaccaataatccaaatccagccatcccaaattggtagtgaatgattataaataacagtaaacagtaatgggccaataacaccggttgcattggtaaggctcaccaataatccctgtaaagcaccttgctcatgactctttgtttggatagacatcactccctgtaatgcaggtaaagcgatcccaccaccagccaataaaattaaaacagggaaatctaaccaaccttcagatataaacgctaaaaaggcaaatgcactactatctgcaataaattcgagcagtactgccgttttttcgccccatttagtggctattcttcctgccacaaaggcttggaatactgagtgtaaaagaccaagacccgctaatgaaaagccaaccatcatgctattccatccaaaacgattttcggtaaatagcacccacaccgttgcgggaatttggcctatcaattgcgctgaaaaataaataatcaacaaaatgggcatcgttttaaataaagtgatgtacaccgaatttgattgcgtctcaacccctacttcggtatctgtattatcacgtgtatttttggtttcacggaaccaaaacataaccacaaggaaagtgacaatatttagcaacgcagcgataaaaaagggactatgcggtgaaatctctcctgcaaaaccaccaataataggccccgctattaaaccaagcccaaaacttgcccctaaccaaccgaaccacttcacgcgttgagaagctgaggtggtatcggcaatgaccgatgccgcgacagccccagtagctcctgtgatccctgaaagcaaacggcctaaatacagcatccaaagcgcacttgaaaaagccagcaataagtaatccagcgatgcgcctattaatgacaacaacagcactgggcgccgaccaaatcggtcagacatttttccaagccaaggagcaaagataacctgcattaacgcataaagtgcaagcaatacgccaaagtggttagcgatatcttccgaagcaataaattcacgtaataacgttggcaagactggcatgataaggccaatccccatggcatcgagtaacgtaattaccaatgcgatctttgtcgaactattcatttcacttttctctatcactgatagggagtgggaaaataactctatcaatgatagggtgtcaaatcgatggcccccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcctgcaggtcgactctagaggatccattccggggatccgtcgacctgcagttcgaagttcctattctctagaaagtataggaacttcagagcgcttttgaagctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctaataaggggatcttgaagttcctattccgaagttcctattctctagaaagtataggaacttcgaagcagctccagcctacagagctcgaattctcatgtttgacagcttatcactgatcagtgaattaatggcgatgacgcatcctcacgataatatccgggtaggcgcaatcactttcgtctctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaagctagaggtgaatcacgacaaagcgtatcaaaaacgtatggagtagggctctaaactctgtataaaaagtttccagctagctgataacgggaaagaaacagagaagggcacaaatattgtgtactttaatgtgccctttaatttattgattggtggttgaattgtccgtaactttttgatttaagtgcaaatttctaataaattagaacactttcttaaatggtttcactgaaacgtgttcatagactcctgccgctacgtacgggtcagcatcggcccaagcctgagctgcttccagcgactcaaattcagcaataacggttgagccagtaaatcccgcagcc

SEQ ID NO: 49 - protein, 28 aa, synthetic construct
  REGION 1..28: note = linker
GSAGSAEAGSNWSHPQFEKGSAGSAAGS

SEQ ID NO: 50 - protein, 12 aa, synthetic construct
  REGION 1..12: note = linker
GSAGSAAGSGEF

SEQ ID NO: 51 - DNA, 84 bp, synthetic construct
  misc_feature 1..84: note = linker
  source 1..84: mol_type = other DNA
ggtagtgctggtagtgctgaagctggtagtaattggagtcatccacaatttgaaaaaggtagtgctggtagtgctgctggtagt

SEQ ID NO: 52 - DNA, 36 bp, synthetic construct
  misc_feature 1..36: note = linker
  source 1..36: mol_type = other DNA
ggtagtgctggtagtgctgctggtagtggtgaattt

SEQ ID NO: 53 - DNA, 3669 bp, Lactobacillus reuteri
  source 1..3669: mol_type = genomic DNA
atgctatcaagaaaaaattataaggaaactatacgaaaacagacacctacaaaacagtactatactattaagaaattaactgttggggttacttcggtattaattggtctatcctttatgggagaactagaaggggatagcgttcatgcggacacgatgacagcaagcagtgagtcaacaagtgttacgtcgacgactgctcaggatggtttaaaaaaatctccacaactctatttgcaagttactgatacaaataacccaagtacaccattaagtgcttcatccacagggactagtaagaatgttacctcatcagctgcggtacaagtgaagtccgctagtgatgaagaagatagtgattctacactagctaagggagaaaataaatttgctcggtcagcagtaaaagattcagtcactgatgggaaaacaagtacagcagaaattaatccggcaaaattaagcagtcctgctttaataacgcaactcaaccaatccttagctaagagcagtacgagtgatgcagcaaaagctaatgatgagttagaaattaaagcaacagatccgactaattatccaaactgtggcgatgtgtatgggccattatttgaattggatgctagcggacagcttgttaataaagatgaagttatatctcttaaagatatgtatattttccaaatattgaaattagtaaatacaaaagatagtgactttcaatatgtaatattaacaatgaatcgtaaagatactgcagataggtctgtatatctttttgtaactggaagcaattatagtaatgctgttgttgttaaagtaaagccaaatgatacttatgaattaagtaaaactggatatagtgttacttatacagaaccaacaactataaatggacattatgttgatggaactttttatgttacaggaagtacttacgatgatggttttataatgccagattggcaactgcagcaccttcagattatatatagtttaggaaattatgatccaagcaatactgacgcaacatcagtttgtgaaataatgccaagttatgaaaaggtaccggtaattaaatatagtggagtaccttcaaatattagccaacctaaggtttacattaccgggtttacgggtcaagagtttaacgttacagatattattaacaattataagaaagtttttaagggctactatcttcaaaatcctaatgtggcgtccatgggaactctttcccaatttgagaatggtggttattacttaaagacatattatgataatgatggtaatgttgactttaagggcttgtatcatcaaattgatgatcagggaacaatgagtgtgagtgttcttaatgcagataataaaacaattgttggacctgaaaatattcttgctggtaaatcgcataactttaactttaatggtcataactggattgcgcggaatccttatgtcactagttcagctcacgaagtcatattaaagtatgctaagttaggttcagttattcctgttgatgaaaacggaaataaaataaacgatggatggcaatatgttaatgatccagatgatgcttccaaagccactagcccatatgaaaaagcgccagttatcgatggttatgtagctgtaaatccagatgaaacgatcgttcttcctcataacttaagtagtgacacaaagatttattaccgaaagaggattaaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgtgactgtaaattacaagggctatgataagaaagtctatgatggtcaacctggaacgattaatccgggtaaattaacgtggagtaagttgccagatggtacttcattgaagatgccaacatggagtatagatgatttcgcttgggaaacagctgatggcttagcaccaacggcagtaggaacttatcggattatcttgacggatgctggtaaggctgcactaaagaagattaatccaaattatgacttaagcagtattactggtgtctttacttatgaaattaagccagcacagacaccagaaatcttaggccaaacacctgagcaacaaccaggccaaaatactaatcaatcaggagctgaaaacggctttggttcttctacaaggcctaatgcatcaactaactccaatcttaatcaacttccacagactggtaatgagcattctaatactgcacttgctggtctagcattggctttcttgactgctatgcttggtttgggcaagaagcgtaaacatgattag

SEQ ID NO: 54 - DNA, 18 bp, synthetic construct
  misc_feature 1..18: note = Primer
  source 1..18: mol_type = other DNA
cggctaccacccaaggaa

SEQ ID NO: 55 - DNA, 18 bp, synthetic construct
  misc_feature 1..18: note = Primer
  source 1..18: mol_type = other DNA
gctggaattaccgcggct

SEQ ID NO: 56 - DNA, 21 bp, synthetic construct
  misc_feature 1..21: note = Primer
  source 1..21: mol_type = other DNA
accgtgaatcttggctgtaac

SEQ ID NO: 57 - DNA, 21 bp, synthetic construct
  misc_feature 1..21: note = Primer
  source 1..21: mol_type = other DNA
gcagcaaatcgcttgggatta

SEQ ID NO: 58 - DNA, 20 bp, synthetic construct
  misc_feature 1..20: note = Primer
  source 1..20: mol_type = other DNA
tgtcagtcatcgcccatgtg

SEQ ID NO: 59 - DNA, 21 bp, synthetic construct
  misc_feature 1..21: note = Primer
  source 1..21: mol_type = other DNA
catccttgcgagtgtcagtga

SEQ ID NO: 60 - DNA, 24 bp, synthetic construct
  misc_feature 1..24: note = Primer
  source 1..24: mol_type = other DNA
catcttctcaaaattcgagtgaca

SEQ ID NO: 61 - DNA, 23 bp, synthetic construct
  misc_feature 1..23: note = Primer
  source 1..23: mol_type = other DNA
tgggagtagacaaggtacaaccc

SEQ ID NO: 62 - DNA, 23 bp, synthetic construct
  misc_feature 1..23: note = Primer
  source 1..23: mol_type = other DNA
tcaagtggcatagatgtggaaga

SEQ ID NO: 63 - DNA, 21 bp, synthetic construct
  misc_feature 1..21: note = Primer
  source 1..21: mol_type = other DNA
tggctctgcaggattttcatg

SEQ ID NO: 64 - DNA, 20 bp, synthetic construct
  misc_feature 1..20: note = Primer
  source 1..20: mol_type = other DNA
agggccctttgctatggtgt

SEQ ID NO: 65 - DNA, 20 bp, synthetic construct
  misc_feature 1..20: note = Primer
  source 1..20: mol_type = other DNA
tggccacagttttcagggat

SEQ ID NO: 66 - DNA, 20 bp, synthetic construct
  misc_feature 1..20: note = Primer
  source 1..20: mol_type = other DNA
agcttccttgtgcaagtgtc

SEQ ID NO: 67 - DNA, 20 bp, synthetic construct
  misc_feature 1..20: note = Primer
  source 1..20: mol_type = other DNA
cccttcatcttttggggtcc

SEQ ID NO: 68 - DNA, 20 bp, synthetic construct
  misc_feature 1..20: note = Primer
  source 1..20: mol_type = other DNA
tccctctgtgatctgggaag

SEQ ID NO: 69 - DNA, 20 bp, synthetic construct
  misc_feature 1..20: note = Primer
  source 1..20: mol_type = other DNA
ctcgaccctgaaagtgaagg

SEQ ID NO: 70 - DNA, 22 bp, synthetic construct
  misc_feature 1..22: note = Primer
  source 1..22: mol_type = other DNA
cccgtataaccatcaccatcat

SEQ ID NO: 71 - DNA, 21 bp, synthetic construct
  misc_feature 1..21: note = Primer
  source 1..21: mol_type = other DNA
ggcatctttcttggcaacttc

SEQ ID NO: 72 - DNA, 17 bp, synthetic construct
  misc_feature 1..17: note = Primer
  source 1..17: mol_type = other DNA
gccagatcccgaaacca

SEQ ID NO: 73 - DNA, 20 bp, synthetic construct
  misc_feature 1..20: note = Primer
  source 1..20: mol_type = other DNA
tataggagtctcggcagtca